(19) United States
(12) Patent Application Publication
(10) Pub. No.: US 2023/0087422 A1
(43) Pub. Date: Mar. 23, 2023

(54) TREATMENT OF HPV-RELATED DISEASES

(71) Applicant: ISA Pharmaceuticals B.V., Oegstgeest (NL)

(72) Inventors: Thomas Johannes Maria Beenakker, Leiden (NL); Gerben Moolhuizen, Bussum (NL); Cornelis Johannes Maria Melief, Haarlem (NL); Miranda Bernardina Johanna Molenaar, Maarssen (NL); Elvin Irsan Kooi, Leiderdorp (NL); Richard Johannes van Duin, Delft (NL); Thomas Morsch, Amsterdam (NL)

(73) Assignee: ISA Pharmaceuticals B.V., Oegstgeest (NL)

(21) Appl. No.: 17/797,438

(22) PCT Filed: Feb. 5, 2021
(86) PCT No.: PCT/EP2021/052738

§ 371 Date: Aug. 4, 2022

(30) Foreign Application Priority Data
Feb. 7, 2020 (EP) 20156038.0
Jun. 29, 2020 (EP) 20182979.3
Jan. 15, 2021 (EP) 21151858.4

(51) Int. Cl. A61K 39/12; C12Q 1/70; C07K 14/005

(52) U.S. Cl. CPC A61K 39/12; C12Q 1/708; C07K 14/005; C12N 2710/20034; C12N 2710/20022

(57) ABSTRACT

The invention provides methods for treating infections, disorders or diseases caused by a human papillomavirus other than HPV-16 by determining the HPV type of the patient, providing a synthetic-long-peptide based therapeutic vaccine for treatment of said patient and administering said therapeutic vaccine to said patient. The invention further provides novel immunogenic compositions and therapeutic vaccines against human papillomaviruses other than HPV-16 and uses thereof.

### FIELD OF THE INVENTION

The present invention relates to the field of immunology. In particular, it relates to novel methods for treating diseases related to human papilloma virus (HPV). Furthermore, the invention relates to novel vaccines and immunogenic compositions suitable for the treatment of HPV-related diseases and uses of such vaccines and compositions.

### BACKGROUND OF THE INVENTION

Human papillomavirus (HPV) is a DNA virus from the papillomavirus family that can infect human beings. Some HPV infections cause no symptoms and resolve spontaneously. Others can result in warts or in precancerous lesions. Various types of cancer can originate from lesions caused by HPV. Cervical cancer is an example of a very common HPV-related cancer.

Due to the high prevalence and mortality, there is an urgent need to develop an effective therapeutic HPV vaccine for clearance of these infections/cancers. Prophylactic HPV vaccines against most oncogenic HPV types are available, but these do not have effective therapeutic effects on established HPV-related cancers.

The HPV genome encodes oncoproteins, amongst others the early antigens E6 and E7, which are integrated in the host genome and constitutively expressed in high-grade cervical lesions and cancer. E6 and E7 are causative to the onset and required for the maintenance of the malignant cellular phenotype and are therefore candidate tumor rejection antigens.

Several E6 and E7-based therapeutic vaccines, including peptide- and DNA-based, have been developed, see e.g. Manuri et al. (2007) Vaccine 25:3302; Kenter et al. (2008) Clin Cancer Res 14:169; Kenter et al. (2009) N Engl J Med 361:1838; Peng et al. (2007) Clin Cancer Res 13:2479; Trimble et al. (2015) Lancet 386:2078. Work to further improve E6/E7-based vaccines is still ongoing.

WO08/147187 describes various T cell epitopes of the E6 and E7 proteins as well as vaccines based on these epitopes.

ISA101 is an HPV vaccine comprising long peptides based on the E6 and E7 proteins of HPV-16. Vaccination with ISA101 has demonstrated to result in complete and partial regressions of HPV-induced vulvar intraepithelial neoplasia lesion(s), especially in patients who displayed a strong vaccine-induced HPV specific T-cell response (Welters et al. (2010) Proc Natl Acad Sci USA 107:11895; Kenter et al. (2009) N Engl J Med 361:1838.

More than 170 types of HPV have been identified. HPV-16 is the most common cause of cervical cancer (Munoz et al. (2003) N Engl 3 Med 348:518). However, a significant proportion of cancers, such as cervical cancers, is related to less prevalent HPV types, such as HPV-18, HPV-45, HPV-33, HPV-31, HPV-52, HPV-58, HPV-35, HPV-39, HPV-59, HPV-73, HPV-51, HPV-56 and HPV-68. The prevalence of these less common HPV types varies geographically. Sequence identity between the E6 and E7 proteins of HPV types is generally rather low.

While significant progress has been made in the therapeutic vaccination using long peptides against HPV-16 affected cells, there is still a need for methods and compositions for vaccination against less common types of HPV.

### SUMMARY OF THE INVENTION

The present invention provides a method for treating an infection, a disorder or a disease caused by a human papillomavirus other than HPV-16 (HPV type 16) by determining the HPV type of the patient, providing a synthetic long peptide (SLP)-based vaccine for treatment of said patient and administering said vaccine to said patient. The inventors have identified novel SLPs based on other types than HPV-16 that can be manufactured with consistently high yield and purity enabling the production of vaccines “on-demand”, i.e. the SLPs of the invention or mixture thereof can efficiently be manufactured ad hoc when a patient with a rare HPV type is identified. Selection of the optimal vaccine composition and the efficient production of SLPs with high yield and purity is challenging. These challenges have been overcome by the compositions of the present invention.

Accordingly, in a first main aspect, the invention relates to a method for treating an infection, disorder or disease caused by a human papillomavirus type other than HPV-16, comprising the steps of:
a) providing a sample from a human subject suspected of having cells expressing HPV genes,
b) subjecting the sample to an assay to determine whether said human subject has cells expressing HPV genes and, if so, of which HPV type,
c) providing, if the subject has cells expressing HPV genes and the type is not HPV-16, one or more immunogenic composition(s) comprising a plurality of peptides, wherein each of said peptides comprises or consists of a contiguous fragment of 18-95 amino acids in length of the sequence of the E6 or the E7 protein of the HPV type identified in the sample, wherein the contiguous E6 and E7 fragments comprised within the plurality of peptides together in total cover at least 70% of the combined E6 and E7 sequences, and
d) treating said human subject with said immunogenic composition(s) treating said human subject with said immunogenic composition(s) and/or treating said human subject with a population of antigen-loaded activated antigen presenting cells (APCs) or a population of expanded antigen-specific T cells, wherein said population of cells (i.e. said population of APCs or said population of expanded antigen-specific T cells) has been generated ex vivo using said immunogenic composition(s).

In further aspects, the invention relates to methods for treating an infection, disorder or disease caused by an HPV type other than HPV-16 comprising administering to a human subject a plurality of peptides, wherein each of said peptides comprises or consists of a contiguous fragment of 18-95 amino acids in length of the sequence of the E6 or the E7 protein of said HPV type other than HPV-16, wherein the contiguous E6 and E7 fragments comprised within the plurality of peptides together in total cover at least 70% of the combined E6 and E7 sequences. Specific sets of peptides for specific HPV types are disclosed herein. In further aspects, the invention relates to vaccines and kits comprising such peptides.

In further aspects, the invention relates to an immunogenic composition comprising one or more peptides selected from the group of peptides set forth in SEQ ID NO:1, 2, 3, 130, 48, 50, 52, 54, 56, 58, 60 and 63 and to a method for treating an infection, disorder or disease caused by HPV-18, comprising administration to a subject of one or more peptides selected from the group of peptides set forth in: SEQ ID NO:1, 2, 3, 130, 48, 50, 52, 54, 56, 58, 60 and 63.

### DETAILED DESCRIPTION OF THE INVENTION

### Definitions

The term “HPV-18”, when used herein, refers to HPV type (also termed subtype) 18. UniProtKB—P06463 describes the sequence of the HPV-18 E6 protein, also set forth in SEQ ID NO:126 herein. UniProtKB—P06788 describes the sequence of the HPV-18 E7 protein, also set forth in SEQ ID NO:127 herein.

SEQ ID NO: 126:
MARFEDPTRR PYKLPDLCTE LNTSLQDIEI TCVYCKTVLE
LTEVFEFAFK DLFWVYRDSI PHAACHKCID FYSRIRELRH
YSDSVYGDTL EKLTNTGLYN LLIRCLRCQK PLNPAEKLRH
LNEKRRFHNI AGHYRGQCHS CCNRARQERL QRRRETQV
SEQ ID NO: 127:
MHGPKATLQD IVLHLEPQNE IPVDLLCHEQ LSDSEEENDE
IDGVNHQHLP ARRAEPQRHT MLCMCCKCEA RIKLVVESSA
DDLRAFQQLF LNTLSFVCPW CASQQ

The term “HPV-45”, when used herein, refers to HPV type 45. UniProtKB—P21735 describes the sequence of the HPV-45 E6 protein, also set forth in SEQ ID NO:128 herein. UniProtKB—P21736 describes the sequence of the HPV-45 E7 protein, also set forth in SEQ ID NO:129 herein.

SEQ ID NO: 128:
MARFDDPKQR PYKLPDLCTE LNTSLQDVSI ACVYCKATLE
RTEVYQFAFK DLCIVYRDCI AYAACHKCID FYSRIRELRY
YSNSVYGETL EKITNTELYN LLIRCLRCQK PLNPAEKRRH
LKDKRRFHSI AGQYRGQCNT CCDQARQERL RRRRETQV
SEQ ID NO: 129:
MHGPRETLQE IVLHLEPQNE LDPVDLLCYE QLSESEEEND
EADGVSHAQL PARRAEPQRH KILCVCCKCD GRIELTVESS
AEDLRTLQQL FLSTLSFVCP WCATNQ

The term “HPV-16”, when used herein, refers to HPV type 16. UniProtKB—P03126 describes the sequence of the HPV-16 E6 protein. UniProtKB—P03129 describes the sequence of the HPV-16 E7 protein.

The term “HPV-33”, when used herein, refers to HPV type 33. UniProtKB—P06427 describes the sequence of the HPV-33 E6 protein, also set forth in SEQ ID NO:134 herein. UniProtKB—P06429 describes the sequence of the HPV-33 E7 protein, also set forth in SEQ ID NO:135 herein.

SEQ ID NO: 134:
MFQDTEEKPR TLHDLCQALE TTIHNIELQC VECKKPLQRS
EVYDFAFADL TVVYREGNPF GICKLCLRFL SKISEYRHYN
YSVYGNTLEQ TVKKPLNEIL IRCIICQRPL CPQEKKRHVD
LNKRFHNISG RWAGRCAACW RSRRRETAL
SEQ ID NO: 135:
MRGHKPTLKE YVLDLYPEPT DLYCYEQLSD SSDEDEGLDR
PDGQAQPATA DYYIVTCCHT CNTTVRLCVN STASDLRTIQ
QLLMGTVNIV CPTCAQQ

The term “HPV-31”, when used herein, refers to HPV type 31. UniProtKB—P17386 describes the sequence of the HPV-31 E6 protein, also set forth in SEQ ID NO:221 herein. UniProtKB—P17387 describes the sequence of the HPV-31 E7 protein, also set forth in SEQ ID NO:222 herein.

SEQ ID NO: 221:
MFKNPAERPR KLHELSSALE IPYDELRLNC VYCKGQLTET
EVLDFAFTDL TIVYRDDTPH GVCTKCLRFY SKVSEFRWYR
YSVYGTTLEK LTNKGICDLL IRCITCQRPL CPEEKQRHLD
KKKRFHNIGG RWTGRCIACW RRPRTETQV
SEQ ID NO: 222:
MRGETPTLQD YVLDLQPEAT DLHCYEQLPD SSDEEDVIDS
PAGQAEPDTS NYNIVTFCCQ CKSTLRLCVQ STQVDIRILQ
ELLMGSFGIV CPNCSTRL

The term “HPV-52”, when used herein, refers to HPV type 52. UniProtKB—P36814 describes the sequence of the HPV-52 E6 protein, also set forth in SEQ ID NO:186 herein. UniProtKB—P36831 describes the sequence of the HPV-52 E7 protein, also set forth in SEQ ID NO:187 herein.

SEQ ID NO: 186:
MFEDPATRPR TLHELCEVLE ESVHEIRLQC VQCKKELQRR
EVYKFLFTDL RIVYRDNNPY GVCIMCLRFL SKISEYRHYQ
YSLYGKTLEE RVKKPLSEIT IRCIICQTPL CPEEKERHVN
ANKRFHNIMG RWTGRCSECW RPRPVTQV
SEQ ID NO: 187:
MRGDKATIKD YILDLQPETT DLHCYEQLGD SSDEEDTDGV
DRPDGQAEQA TSNYYIVTYC HSCDSTLRLC IHSTATDLRT
LQQMLLGTLQ WCPGCARL

The term “HPV-58”, when used herein, refers to HPV type 58. UniProtKB—P26555 describes the sequence of the HPV-58 E6 protein. UniProtKB—P26557 describes the sequence of the HPV-58 E7 protein.

The term “HPV-35”, when used herein, refers to HPV type 35. UniProtKB—P27228 describes the sequence of the HPV-35 E6 protein. UniProtKB—P27230 describes the sequence of the HPV-35 E7 protein.

The term “HPV-39”, when used herein, refers to HPV type 39. UniProtKB—P24835 describes the sequence of the HPV-39 E6 protein. UniProtKB—P24837 describes the sequence of the HPV-39 E7 protein.

The term “HPV-51”, when used herein, refers to HPV type 51. UniProtKB—P26554 describes the sequence of the HPV-51 E6 protein. UniProtKB—P26558 describes the sequence of the HPV-51 E7 protein.

The term “HPV-56”, when used herein, refers to HPV type 56. UniProtKB—P24836 describes the sequence of the HPV-56 E6 protein. UniProtKB—P36833 describes the sequence of the HPV-56 E7 protein.

The term “HPV-68”, when used herein, refers to HPV type 68. UniProtKB—P54667 describes the sequence of the HPV-68 E6 protein. UniProtKB—P54668 describes the sequence of the HPV-68 E7 protein.

The term “HPV-59”, when used herein, refers to HPV type 59. UniProtKB—T2A6S8 describes the sequence of the HPV-59 E6 protein. UniProtKB—T2A7D4 describes the sequence of the HPV-59 E7 protein.

The term “HPV-73”, when used herein, refers to HPV type 73. UniProtKB—A0A0P0ELG2 describes the sequence of the HPV-73 E6 protein. UniProtKB—A0A0P0EFU4 describes the sequence of the HPV-73 E7 protein.

The term “HPV-11”, when used herein, refers to HPV type 11. UniProtKB—P04019 describes the sequence of the HPV-11 E6 protein. UniProtKB—P04020 describes the sequence of the HPV-11 E7 protein.

The term “HPV-6”, when used herein, refers to HPV type 6B. UniProtKB—P06462 describes the sequence of the HPV-6B E6 protein. UniProtKB—P06464 describes the sequence of the HPV-6B E7 protein.

The term “HPV-1”, when used herein, refers to HPV type 1A UniProtKB—P06929 describes the sequence of the HPV-1 E6 protein. UniProtKB—P06465 describes the sequence of the HPV-1A E7 protein.

“Treatment” or “treating” refers to the administration of an effective amount of a composition or vaccine with the purpose of easing, ameliorating, arresting, eradicating (curing) or preventing symptoms, disorders or disease states. An “effective amount” refers to an amount effective, at dosages and for periods of time necessary, to achieve a desired therapeutic result.

When used herein, the term “immunogenic composition” means a composition capable of triggering or boosting an immune response.

When used herein, the term “vaccine” means a product for triggering or boosting an immune response. A vaccine may be administered directly to a human subject or may be used in ex vivo immunization regimens. In ex vivo immunization regimens, the vaccine may be used to generate antigen-loaded antigen presenting cells (APCs), such as antigen-loaded activated Dendritic Cells (DCs), and subsequently stimulate expansion of antigen-specific T cells (e.g. CD4 and CD8 positive circulating T cells, Tumor Infiltrating Lymphocytes (TILs)). Such antigen-loaded APCs or expanded antigen-specific T cells are subsequently administered to a human subject. A vaccine may be a single immunogenic composition or comprise more than one immunogenic composition.

Within the context of the present invention “18-95 amino acids in length” means that the number of amino acid residues is from 18 to 95, i.e. 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94 or 95 amino acid residues. Peptides used in the invention, also denominated herein as long peptides, exceed the length of human leukocyte antigen (HLA) class I and class II presented epitopes. Preferably, the long peptides of the invention are synthetic peptides, denominated herein as synthetic long peptides (SLPs).

The term “wherein the each of said peptides comprises or consists of a contiguous fragment of 18-95 amino acids in length of the sequence of the E6 or the E7 protein” means that each of the peptides comprises or consists of a sequence of 18-95 consecutive amino acids that corresponds to a part of the sequence of the E6 or the E7 protein, i.e. is identical to a fragment of the E6 or E7 protein. This does not exclude, however, that the peptide may be further modified, e.g. conjugated, such as covalently bound to another molecule, e.g. an adjuvant. Preferably, the length of the contiguous amino acid sequence from the E6 or E7 is 18-90 amino acids, or 19-80 amino acids, or 25-70 amino acids, or 25-60 amino acids, or 25-50, more preferably 22-40 amino acids, even more preferably 28-40 and even more preferably 30-39 amino acids.

The term “wherein the contiguous E6 and E7 fragments comprised within the plurality of peptides together in total cover at least 70% of the combined E6 and E7 sequences” means that when the contiguous E6 and E7 fragments comprised within the plurality of peptides are aligned with the combined E6 and E7 sequences, less than 30% of the amino acids of the combined E6 and E7 proteins is not comprised within any of the contiguous fragments. “combined E6 and E7 sequences” means the full E6 and full E7 sequences together. For example, the plurality of peptides could cover less than 70% of either E6 or E7, but more than 70% of the other protein, such that at least 70% of the combined sequences is covered.

The term “plurality” means more than one. A vaccine or composition of the invention or used in the method of the invention preferably comprises at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides. “Different peptides” are to be understood herein as having a different amino acid sequence.

The term “overlapping sequences” when used herein in the context of two or more peptides, means that the sequences of the peptides when aligned have an overlapping area wherein the sequence identity between the peptides in the overlapping area is 100%. For example, six peptides in the composition have overlapping sequences if, of the total number of peptides in the composition, there are six peptides that have an overlapping sequence with at least one other peptide in the composition.

“Sequence identity” is herein defined as a relationship between two or more amino acid sequences, as determined by comparing the sequences. Sequence identity can be determined by alignment of two peptide sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman).

Reference to an element by the indefinite article “a” or “an” does not exclude the possibility that more than one of the elements is present, unless the context clearly requires that there be one and only one of the elements. The indefinite article “a” or “an” thus usually means “at least one”.

### Further Aspects and Embodiments of the Invention

As mentioned above, in a first main aspect, the invention relates to a method for treating an infection, disorder or disease caused by a human papillomavirus type other than HPV-16, comprising the steps of:
a) providing a sample from a human subject suspected of having cells expressing HPV genes,
b) subjecting the sample to an assay to determine whether said human subject has cells expressing HPV genes and, if so, of which HPV type,
c) providing, if the subject has cells expressing HPV genes and the type is not HPV-16, one or more immunogenic composition(s) comprising a plurality of peptides, wherein each of said peptides comprises or consists of a contiguous fragment of 18-95 amino acids in length of the sequence of the E6 or the E7 protein of the HPV type identified in the sample, wherein the contiguous E6 and E7 fragments comprised within the plurality of peptides together in total cover at least 70% of the combined E6 and E7 sequences, and
d) treating said human subject with said immunogenic composition(s) and/or treating said human subject with a population of antigen-loaded activated antigen presenting cells (APCs) or a population of expanded antigen-specific T cells, wherein said population of cells has been generated ex vivo using said immunogenic composition(s).

Similarly, in a further aspect, the invention relates to a method for treating an infection, disorder or disease caused by a human papillomavirus type other than HPV-16, comprising the steps of:
a) assaying a sample from a human subject suspected of having cells expressing HPV genes to determine whether said human subject has cells expressing HPV genes and, if so, of which HPV type,
b) providing, if the subject has cells expressing HPV genes and the type is not HPV-16, one or more immunogenic composition(s) comprising a plurality of peptides, wherein each of said peptides comprises or consists of a contiguous fragment of 18-95 amino acids in length of the sequence of the E6 or the E7 protein of the HPV type identified in the sample, wherein the contiguous E6 and E7 fragments comprised within the plurality of peptides together in total cover at least 70% of the combined E6 and E7 sequences, and
c) treating said human subject with said immunogenic composition(s) and/or treating said human subject with a population of antigen-loaded activated antigen presenting cells (APCs) or a population of expanded antigen-specific T cells, wherein said population of cells has been generated ex vivo using said immunogenic composition(s).

Similarly, in a further aspect, the invention relates to a method for treating an infection, disorder or disease caused by a human papillomavirus type other than HPV-16, comprising the steps of:
a) selecting a human subject having cells expressing HPV genes of a type other than HPV-16, and
b) treating said human subject with one or more immunogenic composition(s) comprising a plurality of peptides, wherein the amino acid sequence of each of said peptides comprises or consists of a contiguous fragment of 18-95 amino acids in length of the sequence of the E6 or the E7 protein of the HPV type expressed in cells of said human subject, wherein the contiguous E6 and E7 fragments comprised within the plurality of peptides together in total cover at least 70% of the combined E6 and E7 sequences, and/or treating said human subject with a population of antigen-loaded activated antigen presenting cells (APCs) or a population of expanded antigen-specific T cells, wherein said population of cells has been generated ex vivo using said immunogenic composition(s).

Similarly, in a further aspect, the invention relates to (an) immunogenic composition(s) for use in the treatment of an infection, disorder or disease caused by a human papillomavirus type other than HPV-16, wherein the treatment comprises the steps of:
a) providing a sample from a human subject suspected of having cells expressing HPV genes,
b) subjecting the sample to an assay to determine whether said human subject has cells expressing HPV genes and, if so, of which HPV type,
c) providing, if the subject has cells expressing HPV genes and the type is not HPV-16, one or more immunogenic composition(s) comprising a plurality of peptides, wherein each of said peptides comprises or consists of a contiguous fragment of 18-95 amino acids in length of the sequence of the E6 or the E7 protein of the HPV type identified in the sample, wherein the contiguous E6 and E7 fragments comprised within the plurality of peptides together in total cover at least 70% of the combined E6 and E7 sequences, and
d) treating said human subject with said immunogenic composition(s) and/or treating said human subject with a population of antigen-loaded activated antigen presenting cells (APCs) or a population of expanded antigen-specific T cells, wherein said population of cells has been generated ex vivo using said immunogenic composition(s).

In one embodiment, the step of providing a sample does not comprise a step practiced on the human or animal body. In one embodiment, the sample is a tissue sample, e.g. a cervical cell swap or an oropharyngeal tissue sample or a tumor biopsy, freshly frozen or preferentially a formalin fixed paraffin embedded tissue preparation.

The assay performed on the sample may be any assay suitable for determining HPV gene expression and determining the type of HPV. In one embodiment, the assay involves amplification of DNA or RNA. Many suitable assays have been described in the art, such as those used in Navarro-Vidal et al. (2018) Asian Pac 3 Cancer Prev 19:2417; Chen et al. (2018) 3 Low Genit Tract Dis 22:355; Lehtinen (2018) Int 3 Cancer 143:2299.

### Peptides Used in the Compositions and Methods of the Invention

The peptides used in the present invention are preferably immunogenic peptides. Preferably, said peptides are capable of inducing a potent combined antigen-directed CD4+T helper and CD8+ cytotoxic T cell response, when administered to a human subject. The peptides may be predicted to be immunogenic and/or may be proven to be immunogenic using in vitro or ex vivo assays or by doing in vivo tests appreciated in the art to establish immunogenicity. Preferably, the peptides can be used effectively in the prevention, partial clearance and/or treatment or full clearance of an HPV-related disease or condition in a subject, preferably as detectable by:
activation or an induction of the immune system and/or an increase in antigen-specific activated CD4+ and/or CD8+ T-cells in peripheral blood or in tissues as established by Elispot assay or by tetramer staining of CD4+ or CD8+ T cells or an increase of the cytokines produced by these T-cells as established by intracellular cytokine staining of CD4+ and CD8+ T cells in flow cytometry after at least one week of treatment; and/or
inhibition of proliferation of antigen-related infection or a detectable decrease of antigen-expressing cells or a decrease in cell viability of antigen-expressing cells; and/or
induction or increased induction of cell death of antigen expressing cells; and/or
inhibition or prevention of the increase of antigen-expressing cells; and/or
decrease of viral load (premalignant stages of the disease); and/or
decrease of the reference lesion size; and/or
decrease of the reference tumor size; and/or
increase of survival rates (e.g. Progression Free Survival, Overall Survival).

In a preferred embodiment, the vaccine or (an) immunogenic composition(s) used in the method of the invention comprise(s) a combination of peptides wherein said combination of peptides comprises epitopes capable of binding to at least 70%, 80%, 90%, 92%, 94%, 95%, 96%, 97%, 98%, 99% or 100% of the HLA class I molecules that are encoded by HLA alleles predominant in the population of human subjects to be treated. Preferred HLA class I epitopes in peptides according to the invention are epitopes capable of binding to: HLA-A0101; HLA-A0201; HLA-A0206; HLA-A0301; HLA-A1101; HLAA2301; HLA-A2402; HLA-A2501; HLA-A2601; HLA-A2902; HLA-A3001; HLAA3002; HLA-A3101; HLA-A3201; HLA-A3303; HLA-A6801; HLA-A6802; HLAA7401; HLA-B0702; HLA-B0801; HLA-B1301; HLA-B1302; HLA-B1402; HLAB1501; HLA-B1502; HLA-B1525; HLA-B1801; HLA-B2702; HLA-B2705; HLAB3501; HLA-B3503; HLA-B3701; HLA-B3801; HLA-B3901; HLA-B4001; HLAB4002; HLA-B4402; HLA-B4403; HLA-B4601; HLA-B4801; HLA-B4901; HLAB5001; HLA-B5101; HLA-B5201; HLA-B5301; HLA-B5501; HLA-B5601; HLAB5701; HLA-B5801 and HLA-B5802. In a preferred embodiment, the vaccine or (an) immunogenic composition(s) used in the method of the invention comprise(s) a combination of peptides wherein said combination of peptides comprises epitopes capable of binding to at least 70%, 80%, 90%, 92%, 94%, 95%, 96%, 97%, 98%, 99% or 100% of the HLA class I and epitopes capable of binding to at least 20%, 30%, 40%, 42%, 44%, 45%, 46%, 47%, 48%, 49% or 50% of the HLA class II molecules that are encoded by HLA alleles predominant in the population of human subjects to be treated. In a preferred embodiment, a peptide used in the invention comprises a CTL epitope that shows binding affinity, preferably at least intermediate binding affinity, more preferably high binding affinity to an HLA class I molecules that is encoded by an HLA allele predominant in the population of human subjects to be treated. Preferably, a peptide used in the invention comprises a CTL epitope that shows binding affinity, preferably at least intermediate binding affinity, more preferably high binding affinity to at least one HLA class I molecule of the group of HLA class I molecules consisting of: HLA-A0101; HLA-A0201; HLA-A0206; HLA-A0301; HLA-A1101; HLA-A2301; HLA-A2402; HLA-A2501; HLA-A2601; HLA-A2902; HLA-A3001; HLA-A3002; HLA-A3101; HLA-A3201; HLA-A3303; HLA-A6801; HLA-A6802; HLA-A7401; HLA-B0702; HLA-B0801; HLA-B1301; HLA-B1302; HLA-B1402; HLA-B1501; HLA-B1502; HLA-B1525; HLA-B1801; HLA-B2702; HLA-B2705; HLA-B3501; HLA-B3503; HLA-B3701; HLA-B3801; HLA-B3901; HLA-B4001; HLA-B4002; HLA-B4402; HLA-B4403; HLA-B4601; HLA-B4801; HLA-B4901; HLA-B5001; HLA-B5101; HLA-B5201; HLA-B5301; HLA-B5501; HLA-B5601; HLA-B5701; HLA-B5801 and HLA-B5802.

In a preferred embodiment, a peptide used in the invention comprises a CTL epitope as described above and a T helper epitope that shows binding affinity, preferably at least intermediate binding affinity, more preferably high binding affinity to an HLA class II molecules that is encoded by an HLA allele predominant in the population of human subjects to be treated.

In a preferred embodiment, peptides used in the invention do not have a cysteine residue at the N- or C-terminus of the peptide. Thus, in a preferred embodiment, the vaccine or immunogenic composition(s) do(es) not contain any peptide having cysteine residue at its N- or C-terminus.

Furthermore, in another preferred embodiment, peptides used in the invention do not comprise more than two cysteine residues. Thus, in a preferred embodiment, the vaccine or immunogenic composition(s) do(es) not contain any peptide having a sequence comprising more than two cysteines.

In another preferred embodiment, peptides used in the invention do not comprise more than three methionines. Thus, in a preferred embodiment, the vaccine or immunogenic composition(s) do(es) not contain any peptide having a sequence comprising more than three methionines.

In another preferred embodiment, peptides used in the invention do not have a glutamine at the N-terminus. Thus, in a preferred embodiment, the vaccine or immunogenic composition(s) do(es) not contain any peptide having a glutamine at the N-terminus.

Preferably, a peptide used in the invention, is an isolated peptide, wherein “isolated” does not reflect the extent to which the peptide is purified, but indicates that the peptide has been removed from its natural milieu (i.e., that has been subject to human manipulation), and may be a recombinantly produced peptide or a synthetically produced peptide.

The peptides used in the invention may comprise a non-naturally occurring sequence as a result of comprising additional amino acids not originating from the E6 or E7 protein antigen and/or as a result of comprising a modified amino acid and/or a non-naturally occurring amino acid and/or a covalently linked functional group such as a fluorinated group, a fluorocarbon group, a human toll-like receptor ligand and/or agonist, an oligonucleotide conjugate, PSA, a sugar chains or glycan, a pam3cys and/or derivative thereof preferably such as described in WO2013051936A1, CpG oligodeoxynucleotides (CpG-ODNs), Cyclic dinucleotides (CDNs), a DC pulse cassette, a tetanus toxin derived peptide, a human HMGB1 derived peptide. The peptide of the invention may comprise aminobutyric acid (Abu, an isostere of cysteine). A cysteine of the peptide of the invention may be replaced by Abu.

Peptides are typically produced synthetically. This may be done by solid phase peptide synthesis, e.g. as described in the Examples herein, or by any other suitable method.

In one embodiment of the method of the invention, said plurality of peptides comprises 5, 6, 7, 8, 9, 10 or 11 E6 peptides and 2, 3, 4, 5, 6 or 7 E7 peptides

In a further embodiment, each of the peptides is between 19 and 35 amino acids in length.

In a further embodiment, two or more, e.g. three or more, such as four or more, e.g. five or more, such as six or more, or all of the peptides have overlapping sequences with one or more other peptides in the plurality of peptides, preferably wherein the overlapping sequences at least 10 amino acids, such as at least 12 amino acids, e.g. at least 14 amino acids.

In a further embodiment, the contiguous E6 and E7 fragments comprised within the plurality of peptides together in total cover at least 75% of the E6 sequence and at least 75% of the E7 sequence, e.g. at least 80% of the E6 sequence and at least 80% of the E7 sequence, such as at least 85% of the E6 sequence and at least 85% of the E7 sequence, e.g. at least 90% of the E6 sequence and at least 90% of the E7 sequence, such as at least 92% of the E6 sequence and at least 92% of the E7 sequence, such as at least 92% of the E6 sequence and at least 94% of the E7 sequence, e.g. between 92% and 98% of the E6 sequence and between 94% and 98% of the E7 sequence.

In a further embodiment, at least one peptide, or at least one mixture of peptides or at least one immunogenic composition provided in step c) is manufactured after the determination of the type in step b), wherein “after” means at a later point in time, i.e. subsequent in time to step b). Thus, in one embodiment, in step c), at least one or all of the compositions is/are prepared ad hoc, i.e. when it is known which HPV type the cells of the human subject express.

In a further embodiment, the non-HPV-16 type is selected from the group consisting of HPV-18, HPV-45, HPV-33, HPV-31, HPV-52, HPV-58, HPV-35, HPV-39, HPV-59, HPV-73, HPV-51, HPV-56, HPV-68, HPV-6, HPV-11 and HPV-1, e.g. selected from the group consisting of HPV-45, HPV-33, HPV-31, HPV-52, HPV-58, HPV-35, HPV-39, HPV-59, HPV-73, HPV-51, HPV-56, HPV-68, HPV-6, HPV-11 and HPV-1.

In one embodiment, said HPV other than HPV-16 is HPV-18 and said composition(s) comprise(s):

i) the peptide set forth in SEQ ID NO:1; or
ii) the peptide set forth in SEQ ID NO:2; or
iii) the peptide set forth in SEQ ID NO:3.

In another embodiment, said HPV other than HPV-16 is HPV-18 and said composition(s) comprise(s) the peptide set forth in SEQ ID NO:130.

In another embodiment, said HPV other than HPV-16 is HPV-18 and said composition(s) comprise(s):

i) one, two, three or all of the peptides set forth in SEQ ID NO:4, 5, 6 and 7; or
ii) one, two, three, four, five or all of the peptides set forth in SEQ ID NO:8, 9, 10, 11, 12 and 13; or
iii) one, two, three, four, five or all of the peptides set forth in SEQ ID NO:14, 15, 16, 17, 18 and 19; or
iv) one, two, three, four, five or all of the peptides set forth in SEQ ID NO:20, 21, 22, 23, 24 and 25; or
v) one, two, three, four or all of the peptides set forth in SEQ ID NO:26, 27, 28, 29 and 30, or
vi) one, two, three, four or all of the peptides set forth in SEQ ID NO:26, 131, 28, 29 and 30.

In another embodiment, said HPV other than HPV-16 is HPV-18 and said composition(s) comprise(s):

i) the peptide set forth in SEQ ID NO:31; or
ii) the peptide set forth in SEQ ID NO:32.

In another embodiment, said HPV other than HPV-16 is HPV-18 and said composition(s) comprise(s):

i) one, two or all of the peptides set forth in SEQ ID NO:33, 34 and 35; or
ii) one, two or all of the peptides set forth in SEQ ID NO:36, 37 and 38; or
iii) one, two or all of the peptides set forth in SEQ ID NO:39, 40 and 41; or
iv) one, two or all of the peptides set forth in SEQ ID NO:42, 43 and 44; or
v) one, two or all of the peptides set forth in SEQ ID NO:45, 46 and 47.

In another embodiment, said HPV other than HPV-16 is HPV-18 and said

i) the peptide set forth in SEQ ID NO:48; or
ii) one or both of the peptides set forth in SEQ ID NO:49 and 50; or
iii) one or both of the peptides set forth in SEQ ID NO:51 and 52; or
iv) one or both of the peptides set forth in SEQ ID NO:53 and 54; or
v) one or both of the peptides set forth in SEQ ID NO:55 and 56; or
vi) one or both of the peptides set forth in SEQ ID NO:57 and 58; or
vii) one or both of the peptides set forth in SEQ ID NO:59 and 60; or
viii) one, two or all of the peptides set forth in SEQ ID NO:61, 62 and 63.

In a further embodiment, said HPV other than HPV-16 is HPV-18 and said composition(s) comprise(s) a plurality of peptides selected from the following sets:
1. the peptides set forth in SEQ ID NO:1, 4, 5, 6, 7 and 31,
2. the peptides set forth in SEQ ID NO:2, 4, 5, 6, 7 and 31,
3. the peptides set forth in SEQ ID NO:3, 4, 5, 6, 7 and 31,
4. the peptides set forth in SEQ ID NO:1, 8, 9, 10, 11, 12, 13 and 31,
5. the peptides set forth in SEQ ID NO:2, 8, 9, 10, 11, 12, 13 and 31,
6. the peptides set forth in SEQ ID NO:3, 8, 9, 10, 11, 12, 13 and 31,
7. the peptides set forth in SEQ ID NO:1, 14, 15, 16, 17, 18, 19 and 31,
8. the peptides set forth in SEQ ID NO:2, 14, 15, 16, 17, 18, 19 and 31,
9. the peptides set forth in SEQ ID NO:3, 14, 15, 16, 17, 18, 19 and 31,
10. the peptides set forth in SEQ ID NO:1, 20, 21, 22, 23, 24, 25 and 31,
11. the peptides set forth in SEQ ID NO:2, 20, 21, 22, 23, 24, 25 and 31,
12. the peptides set forth in SEQ ID NO:3, 20, 21, 22, 23, 24, 25 and 31,
13. the peptides set forth in SEQ ID NO:1, 26, 27, 28, 29, 30 and 31,
14. the peptides set forth in SEQ ID NO:2, 26, 27, 28, 29, 30 and 31,
15. the peptides set forth in SEQ ID NO:3, 26, 27, 28, 29, 30 and 31,
16. the peptides set forth in SEQ ID NO:1, 4, 5, 6, 7 and 32,
17. the peptides set forth in SEQ ID NO:2, 4, 5, 6, 7 and 32,
18. the peptides set forth in SEQ ID NO:3, 4, 5, 6, 7 and 32,
19. the peptides set forth in SEQ ID NO:1, 8, 9, 10, 11, 12, 13 and 32,
20. the peptides set forth in SEQ ID NO:2, 8, 9, 10, 11, 12, 13 and 32,
21. the peptides set forth in SEQ ID NO:3, 8, 9, 10, 11, 12, 13 and 32,
22. the peptides set forth in SEQ ID NO:1, 14, 15, 16, 17, 18, 19 and 32,
23. the peptides set forth in SEQ ID NO:2, 14, 15, 16, 17, 18, 19 and 32,
24. the peptides set forth in SEQ ID NO:3, 14, 15, 16, 17, 18, 19 and 32,
25. the peptides set forth in SEQ ID NO:1, 20, 21, 22, 23, 24, 25 and 32,
26. the peptides set forth in SEQ ID NO:2, 20, 21, 22, 23, 24, 25 and 32,
27. the peptides set forth in SEQ ID NO:3, 20, 21, 22, 23, 24, 25 and 32,
28. the peptides set forth in SEQ ID NO:1, 26, 27, 28, 29, 30 and 32,
29. the peptides set forth in SEQ ID NO:2, 26, 27, 28, 29, 30 and 32,
30. the peptides set forth in SEQ ID NO:3, 26, 27, 28, 29, 30 and 32,
31. the peptides set forth in SEQ ID NO:1, 26, 131, 28, 29 and 30,
32. the peptides set forth in SEQ ID NO:2, 26, 131, 28, 29 and 30,
33. the peptides set forth in SEQ ID NO:3, 26, 131, 28, 29 and 30,
170. the peptides set forth in SEQ ID NO:1, 26, 131, 28, 29, 30 and 31,
171. the peptides set forth in SEQ ID NO:2, 26, 131, 28, 29, 30 and 31,
172. the peptides set forth in SEQ ID NO:3, 26, 131, 28, 29, 30 and 31,
173. the peptides set forth in SEQ ID NO:1, 26, 131, 28, 29, 30 and 32,
174. the peptides set forth in SEQ ID NO:2, 26, 131, 28, 29, 30 and 32,
175. the peptides set forth in SEQ ID NO:3, 26, 131, 28, 29, 30 and 32,
176. the peptides set forth in SEQ ID NO:1, 130, 26, 131, 28, 29, 30 and 31,
177. the peptides set forth in SEQ ID NO:2, 130, 26, 131, 28, 29, 30 and 31,
178. the peptides set forth in SEQ ID NO:3, 130, 26, 131, 28, 29, 30 and 31,
179. the peptides set forth in SEQ ID NO:1, 130, 26, 131, 28, 29, 30 and 32,
180. the peptides set forth in SEQ ID NO:2, 130, 26, 131, 28, 29, 30 and 32,
181. the peptides set forth in SEQ ID NO:3, 130, 26, 131, 28, 29, 30 and 32.

In a further embodiment, said HPV other than HPV-16 is HPV-18 and said composition(s) comprise(s) a plurality of peptides selected from the following sets:
34. the peptides set forth in SEQ ID NO:33, 34, 35 and 48,
35. the peptides set forth in SEQ ID NO:36, 37, 38 and 48,
36. the peptides set forth in SEQ ID NO:39, 40, 41 and 48,
37. the peptides set forth in SEQ ID NO:42, 43, 44 and 48,
38. the peptides set forth in SEQ ID NO:45, 46, 47 and 48,
39. the peptides set forth in SEQ ID NO:33, 34, 35, 49 and 50,
40. the peptides set forth in SEQ ID NO:36, 37, 38, 49 and 50,
41. the peptides set forth in SEQ ID NO:39, 40, 41, 49 and 50,
42. the peptides set forth in SEQ ID NO:42, 43, 44, 49 and 50,
43. the peptides set forth in SEQ ID NO:45, 46, 47, 49 and 50,
44. the peptides set forth in SEQ ID NO:33, 34, 35, 51 and 52,
45. the peptides set forth in SEQ ID NO:36, 37, 38, 51 and 52,
46. the peptides set forth in SEQ ID NO:39, 40, 41, 51 and 52,
47. the peptides set forth in SEQ ID NO:42, 43, 44, 51 and 52,
48. the peptides set forth in SEQ ID NO:45, 46, 47, 51 and 52,
49. the peptides set forth in SEQ ID NO:33, 34, 35, 53 and 54,
50. the peptides set forth in SEQ ID NO:36, 37, 38, 53 and 54,
51. the peptides set forth in SEQ ID NO:39, 40, 41, 53 and 54,
52. the peptides set forth in SEQ ID NO:42, 43, 44, 53 and 54,
53. the peptides set forth in SEQ ID NO:45, 46, 47, 53 and 54,
54. the peptides set forth in SEQ ID NO:33, 34, 35, 55 and 56,
55. the peptides set forth in SEQ ID NO:36, 37, 38, 55 and 56,
56. the peptides set forth in SEQ ID NO:39, 40, 41, 55 and 56,
57. the peptides set forth in SEQ ID NO:42, 43, 44, 55 and 56,
58. the peptides set forth in SEQ ID NO:45, 46, 47, 55 and 56,
59. the peptides set forth in SEQ ID NO:33, 34, 35, 57 and 58,
60. the peptides set forth in SEQ ID NO:36, 37, 38, 57 and 58,
61. the peptides set forth in SEQ ID NO:39, 40, 41, 57 and 58,
62. the peptides set forth in SEQ ID NO:42, 43, 44, 57 and 58,
63. the peptides set forth in SEQ ID NO:45, 46, 47, 57 and 58,
64. the peptides set forth in SEQ ID NO:33, 34, 35, 59 and 60,
65. the peptides set forth in SEQ ID NO:36, 37, 38, 59 and 60,
66. the peptides set forth in SEQ ID NO:39, 40, 41, 59 and 60,
67. the peptides set forth in SEQ ID NO:42, 43, 44, 59 and 60,
68. the peptides set forth in SEQ ID NO:45, 46, 47, 59 and 60,
69. the peptides set forth in SEQ ID NO:33, 34, 35, 61, 62 and 63,
70. the peptides set forth in SEQ ID NO:36, 37, 38, 61, 62 and 63,
71. the peptides set forth in SEQ ID NO:39, 40, 41, 61, 62 and 63,
72. the peptides set forth in SEQ ID NO:42, 43, 44, 61, 62 and 63,
73. the peptides set forth in SEQ ID NO:45, 46, 47, 61, 62 and 63.

In another embodiment, said HPV other than HPV-16 is HPV-45 and said composition(s) comprise(s):

i) the peptide set forth in SEQ ID NO:64; or
ii) the peptide set forth in SEQ ID NO:65; or
iii) the peptide set forth in SEQ ID NO:66, or
iv) the peptide set forth in SEQ ID NO:67.

In another embodiment, said HPV other than HPV-16 is HPV-45 and said composition(s) comprise(s):

i) the peptide set forth in SEQ ID NO:68; or
ii) the peptide set forth in SEQ ID NO:69; or
iii) the peptide set forth in SEQ ID NO:70; or
iv) the peptide set forth in SEQ ID NO:71.

In another embodiment, said HPV other than HPV-16 is HPV-45 and said

i) one, two, three or all of the peptides set forth in SEQ ID NO:72, 73, 74 and 75; or
ii) one, two, three, four, five or all of the peptides set forth in SEQ ID NO:76, 77, 78, 79, 80 and 81; or
iii) one, two, three, four or all of the peptides set forth in SEQ ID NO:82, 83, 84 and 85; or
iv) one, two, three or all of the peptides set forth in SEQ ID NO:86, 87, 88 and 89.

In another embodiment, said HPV other than HPV-16 is HPV-45 and said composition(s) comprise(s):

i) the peptide set forth in SEQ ID NO:90.

In another embodiment, said HPV other than HPV-16 is HPV-45 and said composition(s) comprise(s):

i) one, two or all of the peptides set forth in SEQ ID NO:91, 92 and 93; or
ii) one, two or all of the peptides set forth in SEQ ID NO:94, 95 and 96; or
iii) one, two or all of the peptides set forth in SEQ ID NO:97, 98 and 99; or
iv) one, two, three or all of the peptides set forth in SEQ ID NO:100, 101, 102 and 103,
v) one or both of the peptides set forth in SEQ ID NO:104 and 105, or
vi) one, two, three or all of the peptides set forth in SEQ ID NO:106, 107, 108 and 109, or
vii) one, two or all of the peptides set forth in SEQ ID NO: 93, 104 and 132, or
viii) one, two or all of the peptides set forth in SEQ ID NO: 93, 104 and 133.

In another embodiment, said HPV other than HPV-16 is HPV-45 and said composition(s) comprise(s):

i) one or both of the peptides set forth in SEQ ID NO:110 and 111; or
ii) one, two or all of the peptides set forth in SEQ ID NO:112, 113 and 114; or
iii) one, two or all of the peptides set forth in SEQ ID NO:115, 116 and 117, or
iv) one or both of the peptides set forth in SEQ ID NO:118 and 119.

In a further embodiment, said HPV other than HPV-16 is HPV-45 and said composition(s) comprise(s) a plurality of peptides selected from the following sets:
74. the peptides set forth in SEQ ID NO:64, 68, 72, 73, 74, 75 and 90,
75. the peptides set forth in SEQ ID NO:65, 68, 72, 73, 74, 75 and 90,
76. the peptides set forth in SEQ ID NO:66, 68, 72, 73, 74, 75 and 90,
77. the peptides set forth in SEQ ID NO:67, 68, 72, 73, 74, 75 and 90,
78. the peptides set forth in SEQ ID NO:64, 69, 72, 73, 74, 75 and 90,
79. the peptides set forth in SEQ ID NO:65, 69, 72, 73, 74, 75 and 90,
80. the peptides set forth in SEQ ID NO:66, 69, 72, 73, 74, 75 and 90,
81. the peptides set forth in SEQ ID NO:67, 69, 72, 73, 74, 75 and 90,
82. the peptides set forth in SEQ ID NO:64, 70, 72, 73, 74, 75 and 90,
83. the peptides set forth in SEQ ID NO:65, 70, 72, 73, 74, 75 and 90,
84. the peptides set forth in SEQ ID NO:66, 70, 72, 73, 74, 75 and 90,
85. the peptides set forth in SEQ ID NO:67, 70, 72, 73, 74, 75 and 90,
86. the peptides set forth in SEQ ID NO:64, 71, 72, 73, 74, 75 and 90,
87. the peptides set forth in SEQ ID NO:65, 71, 72, 73, 74, 75 and 90,
88. the peptides set forth in SEQ ID NO:66, 71, 72, 73, 74, 75 and 90,
89. the peptides set forth in SEQ ID NO:67, 71, 72, 73, 74, 75 and 90,
90. the peptides set forth in SEQ ID NO:64, 68, 76, 77, 78, 79, 80, 81 and 90,
91. the peptides set forth in SEQ ID NO:65, 68, 76, 77, 78, 79, 80, 81 and 90,
92. the peptides set forth in SEQ ID NO:66, 68, 76, 77, 78, 79, 80, 81 and 90,
93. the peptides set forth in SEQ ID NO:67, 68, 76, 77, 78, 79, 80, 81 and 90,
94. the peptides set forth in SEQ ID NO:64, 69, 76, 77, 78, 79, 80, 81 and 90,
95. the peptides set forth in SEQ ID NO:65, 69, 76, 77, 78, 79, 80, 81 and 90,
96. the peptides set forth in SEQ ID NO:66, 69, 76, 77, 78, 79, 80, 81 and 90,
97. the peptides set forth in SEQ ID NO:67, 69, 76, 77, 78, 79, 80, 81 and 90,
98. the peptides set forth in SEQ ID NO:64, 70, 76, 77, 78, 79, 80, 81 and 90,
99. the peptides set forth in SEQ ID NO:65, 70, 76, 77, 78, 79, 80, 81 and 90,
100. the peptides set forth in SEQ ID NO:66, 70, 76, 77, 78, 79, 80, 81 and 90,
101. the peptides set forth in SEQ ID NO:67, 70, 76, 77, 78, 79, 80, 81 and 90,
102. the peptides set forth in SEQ ID NO:64, 71, 76, 77, 78, 79, 80, 81 and 90,
103. the peptides set forth in SEQ ID NO:65, 71, 76, 77, 78, 79, 80, 81 and 90,
104. the peptides set forth in SEQ ID NO:66, 71, 76, 77, 78, 79, 80, 81 and 90,
105. the peptides set forth in SEQ ID NO:67, 71, 67, 77, 78, 79, 80, 81 and 90,
106. the peptides set forth in SEQ ID NO:64, 68, 82, 83, 84, 85 and 90,
107. the peptides set forth in SEQ ID NO:65, 68, 82, 83, 84, 85 and 90,
108. the peptides set forth in SEQ ID NO:66, 68, 82, 83, 84, 85 and 90,
109. the peptides set forth in SEQ ID NO:67, 68, 82, 83, 84, 85 and 90,
110. the peptides set forth in SEQ ID NO:64, 69, 82, 83, 84, 85 and 90,
111. the peptides set forth in SEQ ID NO:65, 69, 82, 83, 84, 85 and 90,
112. the peptides set forth in SEQ ID NO:66, 69, 82, 83, 84, 85 and 90,
113. the peptides set forth in SEQ ID NO:67, 69, 82, 83, 84, 85 and 90,
114. the peptides set forth in SEQ ID NO:64, 70, 82, 83, 84, 85 and 90,
115. the peptides set forth in SEQ ID NO:65, 70, 82, 83, 84, 85 and 90,
116. the peptides set forth in SEQ ID NO:66, 70, 82, 83, 84, 85 and 90,
117. the peptides set forth in SEQ ID NO:67, 70, 82, 83, 84, 85 and 90,
118. the peptides set forth in SEQ ID NO:64, 71, 82, 83, 84, 85 and 90,
119. the peptides set forth in SEQ ID NO:65, 71, 82, 83, 84, 85 and 90,
120. the peptides set forth in SEQ ID NO:66, 71, 82, 38, 84, 85 and 90,
121. the peptides set forth in SEQ ID NO:67, 71, 82, 83, 84, 85 and 90,
122. the peptides set forth in SEQ ID NO:64, 68, 86, 87, 88, 89 and 90,
123. the peptides set forth in SEQ ID NO:65, 68, 86, 87, 88, 89 and 90,
124. the peptides set forth in SEQ ID NO:66, 68, 86, 87, 88, 89 and 90,
125. the peptides set forth in SEQ ID NO:67, 68, 86, 87, 88, 89 and 90,
126. the peptides set forth in SEQ ID NO:64, 69, 86, 87, 88, 89 and 90,
127. the peptides set forth in SEQ ID NO:65, 69, 86, 87, 88, 89 and 90,
128. the peptides set forth in SEQ ID NO:66, 69, 86, 87, 88, 89 and 90,
129. the peptides set forth in SEQ ID NO:67, 69, 86, 87, 88, 89 and 90,
130. the peptides set forth in SEQ ID NO:64, 70, 86, 87, 88, 89 and 90,
131. the peptides set forth in SEQ ID NO:65, 70, 86, 87, 88, 89 and 90,
132. the peptides set forth in SEQ ID NO:66, 70, 86, 87, 88, 89 and 90,
133. the peptides set forth in SEQ ID NO:67, 70, 86, 87, 88, 89 and 90,
134. the peptides set forth in SEQ ID NO:64, 71, 86, 87, 88, 89 and 90,
135. the peptides set forth in SEQ ID NO:65, 71, 86, 87, 88, 89 and 90,
136. the peptides set forth in SEQ ID NO:66, 71, 86, 87, 88, 89 and 90,
137. the peptides set forth in SEQ ID NO:67, 71, 86, 87, 88, 89 and 90.

In a further embodiment, said HPV other than HPV-16 is HPV-45 and said composition(s) comprise(s) a plurality of peptides selected from the following sets:
138. the peptides set forth in SEQ ID NO:91, 92, 93, 110 and 111,
139. the peptides set forth in SEQ ID NO:94, 95, 96, 110 and 111,
140. the peptides set forth in SEQ ID NO:97, 98, 99, 110 and 111,
141. the peptides set forth in SEQ ID NO:100, 101, 102, 103, 110 and 111,
142. the peptides set forth in SEQ ID NO:104, 105, 110 and 110,
143. the peptides set forth in SEQ ID NO:106, 107, 108, 109, 110 and 111,
144. the peptides set forth in SEQ ID NO: 93, 104, 132, 110 and 111,
145. the peptides set forth in SEQ ID NO: 93, 104, 133, 110 and 111,
146. the peptides set forth in SEQ ID NO:91, 92, 93, 112, 113 and 114,
147. the peptides set forth in SEQ ID NO:94, 95, 96, 112, 113 and 114,
148. the peptides set forth in SEQ ID NO:97, 98, 99, 112, 113 and 114,
149. the peptides set forth in SEQ ID NO:100, 101, 102, 103, 112, 113 and 114,
150. the peptides set forth in SEQ ID NO:104, 105, 112, 113 and 114,
151. the peptides set forth in SEQ ID NO:106, 107, 108, 109, 112, 113 and 114,
152. the peptides set forth in SEQ ID NO: 93, 104, 132, 112, 113 and 114,
153. the peptides set forth in SEQ ID NO: 93, 104, 133, 112, 113 and 114,
154. the peptides set forth in SEQ ID NO:91, 92, 93, 115, 116 and 117,
155. the peptides set forth in SEQ ID NO:94, 95, 96, 115, 116 and 117,
156. the peptides set forth in SEQ ID NO:97, 98, 99, 115, 116 and 117,
157. the peptides set forth in SEQ ID NO:100, 101, 102, 103, 115, 116 and 117,
158. the peptides set forth in SEQ ID NO:104, 105, 115, 116 and 117,
159. the peptides set forth in SEQ ID NO:106, 107, 108, 109, 115, 116 and 117,
160. the peptides set forth in SEQ ID NO: 93, 104, 132, 115, 116 and 117,
161. the peptides set forth in SEQ ID NO: 93, 104, 133, 115, 116 and 117,
162. the peptides set forth in SEQ ID NO:91, 92, 93, 118 and 119,
163. the peptides set forth in SEQ ID NO:94, 95, 96, 118 and 119,
164. the peptides set forth in SEQ ID NO:97, 98, 99, 118 and 119,
165. the peptides set forth in SEQ ID NO:100, 101, 102, 103, 118 and 119,
166. the peptides set forth in SEQ ID NO:104, 105, 118 and 119,
167. the peptides set forth in SEQ ID NO:106, 107, 108, 109, 118 and 119,
168. the peptides set forth in SEQ ID NO: 93, 104, 132, 118 and 119,
169. the peptides set forth in SEQ ID NO: 93, 104, 133, 118 and 119.

In another embodiment, said HPV other than HPV-16 is HPV-33 and said composition(s) comprise(s):

i) the peptide set forth in SEQ ID NO:136; or
ii) the peptide set forth in SEQ ID NO:137; or
iii) the peptide set forth in SEQ ID NO:138.

In another embodiment, said HPV other than HPV-16 is HPV-33 and said composition(s) comprise(s):

i) one, two, three or all of the peptides set forth in SEQ ID NO:139, 140, 141 and 142; or
ii) one, two, three or all of the peptides set forth in SEQ ID NO:139, 140, 143 and 144; or
iii) one, two, three or all of the peptides set forth in SEQ ID NO:141, 145, 146 and 147; or
iv) one, two, three or all of the peptides set forth in SEQ ID NO:145, 148, 149 and 150; or
v) one, two, three, four or all of the peptides set forth in SEQ ID NO:151, 152, 153, 154 and 155; or
vi) one, two, three, four or all of the peptides set forth in SEQ ID NO:142, 156, 157, 158 and 159.

In another embodiment, said HPV other than HPV-16 is HPV-33 and said composition(s) comprise(s):

i) one or both of the peptides set forth in SEQ ID NO:160 and 161; or
ii) one or both of the peptides set forth in SEQ ID NO:162 and 163; or
iii) one or both of the peptides set forth in SEQ ID NO:164 and 165; or
iv) one or both of the peptides set forth in SEQ ID NO:160 and 162; or
v) one or both of the peptides set forth in SEQ ID NO:161 and 163.

In another embodiment, said HPV other than HPV-16 is HPV-33 and said composition(s) comprise(s):

i) one or both of the peptides set forth in SEQ ID NO:166 and 167; or
ii) one, two or all of the peptides set forth in SEQ ID NO:167, 168 and 169; or
iii) one or both of the peptides set forth in SEQ ID NO:167 and 170; or
iv) one, two or all of the peptides set forth in SEQ ID NO:171, 172 and 173; or
v) one, two or all of the peptides set forth in SEQ ID NO:174, 175 and 176.

In another embodiment, said HPV other than HPV-16 is HPV-33 and said composition(s) comprise(s):

i) one or both of the peptides set forth in SEQ ID NO:177 and 178; or
ii) one or both of the peptides set forth in SEQ ID NO:179 and 180; or
iii) one or both of the peptides set forth in SEQ ID NO:181 and 182; or
iv) one or both of the peptides set forth in SEQ ID NO:183 and 184.

In another embodiment, said HPV other than HPV-16 is HPV-33 and said composition(s) comprise(s) the peptide set forth in SEQ ID NO:185.

In another embodiment, said HPV other than HPV-16 is HPV-52 and said composition(s) comprise(s):

i) the peptide set forth in SEQ ID NO:188; or
ii) the peptide set forth in SEQ ID NO:189; or
iii) the peptide set forth in SEQ ID NO:190.

In another embodiment, said HPV other than HPV-16 is HPV-52 and said composition(s) comprise(s):

i) one, two, three, four, five or all of the peptides set forth in SEQ ID NO:191, 192, 193, 194, 195 and 196; or
ii) one, two, three, four, five or all of the peptides set forth in SEQ ID NO:193, 194, 197, 198, 199 and 200; or
iii) one, two, three, four, five or all of the peptides set forth in SEQ ID NO:195, 196, 201, 202, 203 and 204.

In another embodiment, said HPV other than HPV-16 is HPV-52 and said composition(s) comprise(s):

i) one or both of the peptides set forth in SEQ ID NO:205 and 206; or
ii) one or both of the peptides set forth in SEQ ID NO:206 and 207; or
iii) one or both of the peptides set forth in SEQ ID NO:206 and 208.

In another embodiment, said HPV other than HPV-16 is HPV-52 and said composition(s) comprise(s):

i) one, two or all of the peptides set forth in SEQ ID NO:209, 210 and 211; or
ii) one, two or all of the peptides set forth in SEQ ID NO:210, 212 and 213; or
iii) one, two or all of the peptides set forth in SEQ ID NO:211, 214 and 215.

In another embodiment, said HPV other than HPV-16 is HPV-52 and said composition(s) comprise(s):

i) one or both of the peptides set forth in SEQ ID NO:216 and 217; or
ii) one or both of the peptides set forth in SEQ ID NO:218 and 219; or
iii) one or both of the peptides set forth in SEQ ID NO:219 and 220.

In another embodiment, said HPV other than HPV-16 is HPV-31 and said composition(s) comprise(s):

i) one or both of the peptides set forth in SEQ ID NO:223 and 224; or
ii) one or both of the peptides set forth in SEQ ID NO:225 and 226; or
iii) one or both of the peptides set forth in SEQ ID NO:224 and 226.

In another embodiment, said HPV other than HPV-16 is HPV-31 and said composition(s) comprise(s) the peptide set forth in SEQ ID NO:227.

In another embodiment, said HPV other than HPV-16 is HPV-31 and said composition(s) comprise(s):

i) one, two or all of the peptides set forth in SEQ ID NO:228, 229 and 230; or
ii) one or both of the peptides set forth in SEQ ID NO:231 and 232; or
iii) one, two or all of the peptides set forth in SEQ ID NO:229, 230 and 233.

In another embodiment, said HPV other than HPV-16 is HPV-31 and said composition(s) comprise(s):

i) one or both of the peptides set forth in SEQ ID NO:234 and 235; or
ii) one or both of the peptides set forth in SEQ ID NO:234 and 236; or
iii) one or both of the peptides set forth in SEQ ID NO:235 and 237.

In another embodiment, said HPV other than HPV-16 is HPV-31 and said composition(s) comprise(s):

i) one, two or all of the peptides set forth in SEQ ID NO:238, 239 and 240; or
ii) one or both of the peptides set forth in SEQ ID NO:241 and 242; or
iii) one, two or all of the peptides set forth in SEQ ID NO:239, 240 and 243.

In another embodiment, said HPV other than HPV-16 is HPV-31 and said composition(s) comprise(s):

i) one or both of the peptides set forth in SEQ ID NO:244 and 245; or
ii) the peptide set forth in SEQ ID NO:246; or
iii) the peptide set forth in SEQ ID NO:247; or
iv) one or both of the peptides set forth in SEQ ID NO:248 and 249; or
v) the peptide set forth in SEQ ID NO:250.

### Further Methods of Treatment and Vaccines

Methods of Treatment

In a further main aspect, the invention relates to a method for treating an infection, disorder or disease caused by HPV-18, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,

wherein said plurality of peptides comprises or consists of peptides selected from the group consisting of: SEQ ID NO:1 to 63, 130 and 131,

wherein the contiguous E6 and E7 fragments comprised within the plurality of peptides together in total cover at least 70% of the combined E6 and E7 sequences.

In a further main aspect, the invention relates to a method for treating an infection, disorder or disease caused by HPV-18, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,
wherein said plurality of peptides comprises or consists of:
a) a peptide selected from the group consisting of:

i) the peptide set forth in SEQ ID NO:1,

ii) the peptide set forth in SEQ ID NO:2, and

iii) the peptide set forth in SEQ ID NO:3,

and
b) a combination of peptides selected from the group consisting of:

i) the peptides set forth in SEQ ID NO:4, 5, 6 and 7,

ii) the peptides set forth in SEQ ID NO:8, 9, 10, 11, 12 and 13,

iii) the peptides set forth in SEQ ID NO:14, 15, 16, 17, 18 and 19,

iv) the peptides set forth in SEQ ID NO:20, 21, 22, 23, 24 and 25,

v) the peptides set forth in SEQ ID NO:26, 27, 28, 29 and 30,

vi) the peptides set forth in SEQ ID NO:26, 131, 28, 29 and 30,

and
c) a peptide selected from the group consisting of:

i) the peptide set forth in SEQ ID NO:31, and

ii) the peptide set forth in SEQ ID NO:32,

and
d) a combination of peptides selected from the group consisting of:

i) the peptides set forth in SEQ ID NO:33, 34 and 35,

ii) the peptides set forth in SEQ ID NO:36, 37 and 38,

iii) the peptides set forth in SEQ ID NO:39, 40 and 41,

iv) the peptides set forth in SEQ ID NO:42, 43 and 44, and

v) the peptides set forth in SEQ ID NO:45, 46 and 47,

and
e) the peptide set forth in SEQ ID NO:48, or a combination of peptides selected from the group consisting of:

i) the peptides set forth in SEQ ID NO:49 and 50,

ii) the peptides set forth in SEQ ID NO:51 and 52,

iii) the peptides set forth in SEQ ID NO:53 and 54,

iv) the peptides set forth in SEQ ID NO:55 and 56,

v) the peptides set forth in SEQ ID NO:57 and 58,

vi) the peptides set forth in SEQ ID NO:59 and 60, and

vii) the peptides set forth in SEQ ID NO:61, 62 and 63, optionally, the plurality further comprises the peptide set forth in SEQ ID NO:130.

In a further main aspect, the invention relates to a method for treating an infection, disorder or disease caused by HPV-45, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,

wherein said plurality of peptides comprises or consists of peptides selected from the group consisting of: SEQ ID NO:64 to 119, 132 and 133,

wherein the contiguous E6 and E7 fragments comprised within the plurality of peptides together in total cover at least 70% of the combined E6 and E7 sequences.

In a further main aspect, the invention relates to a method for treating an infection, disorder or disease caused by HPV-45, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,
wherein said plurality of peptides comprises or consists of:
a) a peptide selected from the group consisting of:

i) the peptide set forth in SEQ ID NO:64,

ii) the peptide set forth in SEQ ID NO:65,

iii) the peptide set forth in SEQ ID NO:66, and

iv) the peptide set forth in SEQ ID NO:67,

and
b) a peptide selected from the group consisting of:

i) the peptide set forth in SEQ ID NO:68,

ii) the peptide set forth in SEQ ID NO:69,

iii) the peptide set forth in SEQ ID NO:70, and

iv) the peptide set forth in SEQ ID NO:71,

and
c) a combination of peptides selected from the group consisting of:

i) the peptides set forth in SEQ ID NO:72, 73, 74 and 75,

ii) the peptides set forth in SEQ ID NO:76, 77, 78, 79, 80 and 81,

iii) the peptides set forth in SEQ ID NO:82, 83, 84 and 85, and

iv) the peptides set forth in SEQ ID NO:86, 87, 88 and 89,

and
d) the peptide set forth in SEQ ID NO:90,
and
e) a combination of peptides selected from the group consisting of:

i) the peptides set forth in SEQ ID NO:91, 92 and 93,

ii) the peptides set forth in SEQ ID NO:94, 95 and 96,

iii) the peptides set forth in SEQ ID NO:97, 98 and 99,

iv) the peptides set forth in SEQ ID NO:100, 101, 102 and 103,

v) the peptides set forth in SEQ ID NO:104 and 105,

vi) the peptides set forth in SEQ ID NO:106, 107, 108 and 109,

vii) the peptides set forth in SEQ ID NO: 93, 104 and 132, and

viii) the peptides set forth in SEQ ID NO: 93, 104 and 133,

and
f) a combination of peptides selected from the group consisting of:

i) the peptides set forth in SEQ ID NO:110 and 111,

ii) the peptides set forth in SEQ ID NO:112, 113 and 114,

iii) the peptides set forth in SEQ ID NO:115, 116 and 117, and

iv) the peptides set forth in SEQ ID NO:118 and 119.

In a further main aspect, the invention relates to a method for treating an infection, disorder or disease caused by HPV-33, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,

wherein said plurality of peptides comprises or consists of peptides selected from the group consisting of: SEQ ID NO:136 to 185,

wherein the contiguous E6 and E7 fragments comprised within the plurality of peptides together in total cover at least 70% of the combined E6 and E7 sequences.

In a further main aspect, the invention relates to a method for treating an infection, disorder or disease caused by HPV-33, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,
wherein said plurality of peptides comprises or consists of:
a) a peptide selected from the group consisting of:

i) the peptide set forth in SEQ ID NO:136;

ii) the peptide set forth in SEQ ID NO:137; and

iii) the peptide set forth in SEQ ID NO:138,

and
b) a combination of peptides selected from the group consisting of:

i) the peptides set forth in SEQ ID NO:139, 140, 141 and 142;

ii) the peptides set forth in SEQ ID NO:139, 140, 143 and 144;

iii) the peptides set forth in SEQ ID NO:141, 145, 146 and 147;

iv) the peptides set forth in SEQ ID NO:145, 148, 149 and 150;

v) the peptides set forth in SEQ ID NO:151, 152, 153, 154 and 155; and

vi) the peptides set forth in SEQ ID NO:142, 156, 157, 158 and 159,

and
c) a combination of peptides selected from the group consisting of:

i) the peptides set forth in SEQ ID NO:160 and 161;

ii) the peptides set forth in SEQ ID NO:162 and 163;

iii) the peptides set forth in SEQ ID NO:164 and 165;

iv) the peptides set forth in SEQ ID NO:160 and 162; and

v) the peptides set forth in SEQ ID NO:161 and 163,

and
d) a combination of peptides selected from the group consisting of:

i) the peptides set forth in SEQ ID NO:166 and 167;

ii) the peptides set forth in SEQ ID NO:167, 168 and 169;

iii) the peptides set forth in SEQ ID NO:167 and 170;

iv) the peptides set forth in SEQ ID NO:171, 172 and 173; and

v) the peptides set forth in SEQ ID NO:174, 175 and 176,

and
e) a combination of peptides selected from the group consisting of:

i) the peptides set forth in SEQ ID NO:177 and 178;

ii) the peptides set forth in SEQ ID NO:179 and 180;

iii) the peptides set forth in SEQ ID NO:181 and 182;

iv) the peptides set forth in SEQ ID NO:183 and 184, optionally, the plurality further comprises the peptide set forth in SEQ ID NO:185.

In a further main aspect, the invention relates to a method for treating an infection, disorder or disease caused by HPV-52, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,

wherein said plurality of peptides comprises or consists of peptides selected from the group consisting of: SEQ ID NO:188 to 220,

wherein the contiguous E6 and E7 fragments comprised within the plurality of peptides together in total cover at least 70% of the combined E6 and E7 sequences.

In a further main aspect, the invention relates to a method for treating an infection, disorder or disease caused by HPV-52, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,
wherein said plurality of peptides comprises or consists of:
a) a peptide selected from the group consisting of:

i) the peptide set forth in SEQ ID NO:188;

ii) the peptide set forth in SEQ ID NO:189; and

iii) the peptide set forth in SEQ ID NO:190,

and
b) a combination of peptides selected from the group consisting of:

i) the peptides set forth in SEQ ID NO:191, 192, 193, 194, 195 and 196;

ii) the peptides set forth in SEQ ID NO:193, 194, 197, 198, 199 and 200; and

iii) the peptides set forth in SEQ ID NO:195, 196, 201, 202, 203 and 204,

and
c) a combination of peptides selected from the group consisting of:

i) the peptides set forth in SEQ ID NO:205 and 206;

ii) the peptides set forth in SEQ ID NO:206 and 207; and

iii) the peptides set forth in SEQ ID NO:206 and 208,

and
d) a combination of peptides selected from the group consisting of:

i) the peptides set forth in SEQ ID NO:209, 210 and 211;

ii) the peptides set forth in SEQ ID NO:210, 212 and 213; and

iii) the peptides set forth in SEQ ID NO:211, 214 and 215,

and
e) a combination of peptides selected from the group consisting of:

i) the peptides set forth in SEQ ID NO:216 and 217;

ii) the peptides set forth in SEQ ID NO:218 and 219; and

iii) the peptides set forth in SEQ ID NO:219 and 220.

In a further main aspect, the invention relates to a method for treating an infection, disorder or disease caused by HPV-31, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,

wherein said plurality of peptides comprises or consists of peptides selected from the group consisting of: SEQ ID NO:223 to 250,

wherein the contiguous E6 and E7 fragments comprised within the plurality of peptides together in total cover at least 70% of the combined E6 and E7 sequences.

In a further main aspect, the invention relates to a method for treating an infection, disorder or disease caused by HPV-31, comprising

(i) administering to a human subject (or immunizing a human subject with) a plurality of peptides, and/or
(ii) administering to a human subject a population of antigen-loaded activated antigen presenting cells (APCs) or a population of expanded antigen-specific T cells, wherein said population of cells has been generated ex vivo using a plurality of peptides,
wherein said plurality of peptides comprises or consists of:
a) a combination of peptides selected from the group consisting of:

i) the peptides set forth in SEQ ID NO:223 and 224;

ii) the peptides set forth in SEQ ID NO:225 and 226; and

iii) the peptides set forth in SEQ ID NO:224 and 226,

and
b) a combination of peptides selected from the group consisting of:

i) the peptides set forth in SEQ ID NO:228, 229 and 230;

ii) the peptides set forth in SEQ ID NO:231 and 232; and

iii) the peptides set forth in SEQ ID NO:229, 230 and 233,

and
c) a combination of peptides selected from the group consisting of:

i) the peptides set forth in SEQ ID NO:234 and 235;

ii) the peptides set forth in SEQ ID NO:234 and 236; and

iii) the peptides set forth in SEQ ID NO:235 and 237,

and
d) a combination of peptides selected from the group consisting of:

i) the peptides set forth in SEQ ID NO:238, 239 and 240;

ii) the peptides set forth in SEQ ID NO:241 and 242; and

iii) the peptides set forth in SEQ ID NO:239, 240 and 243, and optionally a peptide or combination of peptides selected from the group consisting of:

i) the peptides set forth in SEQ ID NO:244 and 245;

ii) the peptide set forth in SEQ ID NO:246;

iii) the peptide set forth in SEQ ID NO:247;

iv) the peptides set forth in SEQ ID NO:248 and 249; and

v) the peptide set forth in SEQ ID NO:250,

and optionally the peptide set forth in SEQ ID NO:227.

In this method, the plurality of peptides is typically administered in the form of one or more immunogenic compositions. Embodiments of immunogenic compositions are described herein.

In one embodiment, the method of the invention further comprises administration of an adjuvant. In one embodiment, the adjuvant is an emulsifying adjuvant. In another embodiment, the adjuvant is an oil-based adjuvant. Oil-based adjuvants can be used to form emulsions (e.g. water-in-oil or oil-in-water emulsions) and are appreciated in the art to enhance and direct the immune response. Preferably the oil-based adjuvant is a mineral oil-based adjuvant. In one embodiment, the adjuvant is a Montanide adjuvant. Montanide adjuvants which are based on purified squalene and squalene emulsified with highly purified mannide mono-oleate (e.g. Montanide ISA 25 VG, 28 VG, 35 VG, 50 V, 50 V2, 51 VG, 61 VG, 70 VG, 70 M VG, 71 VG, 720 VG, 760 VG, 763 A VG, 775 VG, 780 VG, 201 VG, 206 VG, 207 VG). More preferably, the oil-based adjuvant is Montanide ISA 51VG (Seppic), which is a mixture of Drakeol VR and mannide monooleate.

A further particularly preferred TLR ligand is a Pam3cys and/or derivative thereof, preferably a Pam3cys lipopeptide or variant or derivative thereof, preferably such as described in WO2013051936A1 (incorporated herein by reference), more preferably U-Pam12 or U-Pam14 a.k.a. AMPLIVANT®, as depicted in the following structures:

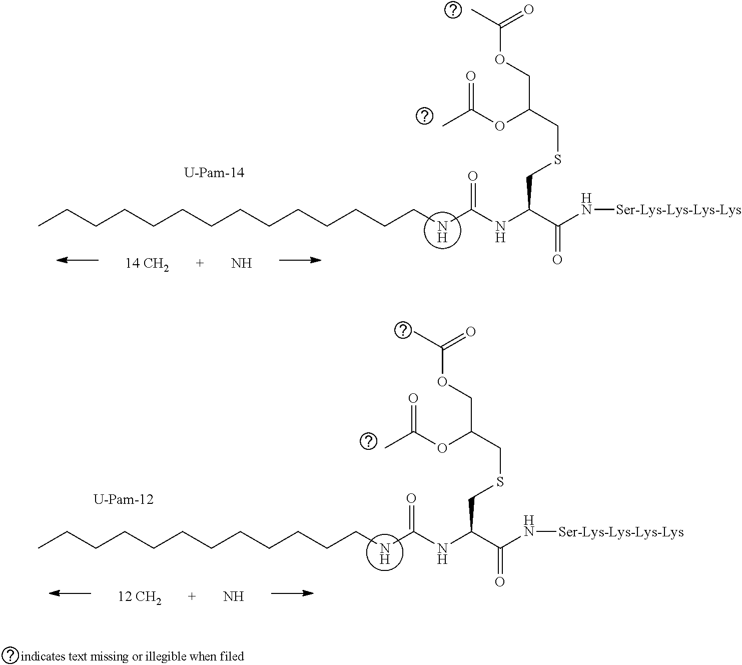

Pam3cys and/or derivatives thereof may optionally be covalently linked to the peptide antigen(s).

In one embodiment, the method is for the treatment of cancer, such as cervical cancer or head-and-neck cancer.

### Vaccines

In a further main aspect, the invention relates to a vaccine comprising a plurality of peptides, wherein said plurality of peptides comprises or consists of peptides selected from the group consisting of: SEQ ID NO:1 to 63, 130 and 131, wherein the contiguous E6 and E7 fragments comprised within the plurality of peptides together in total cover at least 70% of the combined E6 and E7 sequences.

In a further main aspect, the invention relates to a vaccine comprising:

a) a peptide selected from the group consisting of:

i) the peptide set forth in SEQ ID NO:1,

ii) the peptide set forth in SEQ ID NO:2, and

iii) the peptide set forth in SEQ ID NO:3,

and
b) a combination of peptides selected from the group consisting of:

i) the peptides set forth in SEQ ID NO:4, 5, 6 and 7,

ii) the peptides set forth in SEQ ID NO:8, 9, 10, 11, 12 and 13,

iii) the peptides set forth in SEQ ID NO:14, 15, 16, 17, 18 and 19,

iv) the peptides set forth in SEQ ID NO:20, 21, 22, 23, 24 and 25,

v) the peptides set forth in SEQ ID NO:26, 27, 28, 29 and 30,

vi) the peptides set forth in SEQ ID NO:26, 131, 28, 29 and 30,

and
c) a peptide selected from the group consisting of:

i) the peptide set forth in SEQ ID NO:31, and

ii) the peptide set forth in SEQ ID NO:32,

and
d) a combination of peptides selected from the group consisting of:

i) the peptides set forth in SEQ ID NO:33, 34 and 35,

ii) the peptides set forth in SEQ ID NO:36, 37 and 38,

iii) the peptides set forth in SEQ ID NO:39, 40 and 41,

iv) the peptides set forth in SEQ ID NO:42, 43 and 44, and

v) the peptides set forth in SEQ ID NO:45, 46 and 47,

and
e) the peptide set forth in SEQ ID NO:48, or a combination of peptides selected from the group consisting of:

i) the peptides set forth in SEQ ID NO:49 and 50,

ii) the peptides set forth in SEQ ID NO:51 and 52,

iii) the peptides set forth in SEQ ID NO:53 and 54,

iv) the peptides set forth in SEQ ID NO:55 and 56,

v) the peptides set forth in SEQ ID NO:57 and 58,

vi) the peptides set forth in SEQ ID NO:59 and 60, and

vii) the peptides set forth in SEQ ID NO:61, 62 and 63,

optionally, the vaccine further comprises the peptide set forth in SEQ ID NO:130.

In a further main aspect, the invention relates to a vaccine comprising a plurality of peptides, wherein said plurality of peptides comprises or consists of peptides selected from the group consisting of: SEQ ID NO:64 to 119, 132 and 133, wherein the contiguous E6 and E7 fragments comprised within the plurality of peptides together in total cover at least 70% of the combined E6 and E7 sequences.

In a further main aspect, the invention relates to a vaccine comprising:

a) a peptide selected from the group consisting of:

i) the peptide set forth in SEQ ID NO:64,

ii) the peptide set forth in SEQ ID NO:65,

iii) the peptide set forth in SEQ ID NO:66, and

iv) the peptide set forth in SEQ ID NO:67,

and
b) a peptide selected from the group consisting of:

i) the peptide set forth in SEQ ID NO:68,

ii) the peptide set forth in SEQ ID NO:69,

iii) the peptide set forth in SEQ ID NO:70, and

iv) the peptide set forth in SEQ ID NO:71,

and
c) a combination of peptides selected from the group consisting of:

i) the peptides set forth in SEQ ID NO:72, 73, 74 and 75,

ii) the peptides set forth in SEQ ID NO:76, 77, 78, 79, 80 and 81,

iii) the peptides set forth in SEQ ID NO:82, 83, 84 and 85, and

iv) the peptides set forth in SEQ ID NO:86, 87, 88 and 89,

and
d) the peptide set forth in SEQ ID NO:90,
and
e) a combination of peptides selected from the group consisting of:

i) the peptides set forth in SEQ ID NO:91, 92 and 93,

ii) the peptides set forth in SEQ ID NO:94, 95 and 96,

iii) the peptides set forth in SEQ ID NO:97, 98 and 99,

iv) the peptides set forth in SEQ ID NO:100, 101, 102 and 103,

v) the peptides set forth in SEQ ID NO:104 and 105,

vi) the peptides set forth in SEQ ID NO:106, 107, 108 and 109,

vii) the peptides set forth in SEQ ID NO: 93, 104 and 132, and

viii) the peptides set forth in SEQ ID NO: 93, 104 and 133,

and
f) a combination of peptides selected from the group consisting of:

i) the peptides set forth in SEQ ID NO:110 and 111,

ii) the peptides set forth in SEQ ID NO:112, 113 and 114,

iii) the peptides set forth in SEQ ID NO:115, 116 and 117, and

iv) the peptides set forth in SEQ ID NO:118 and 119.

In a further main aspect, the invention relates to a vaccine comprising a plurality of peptides, wherein said plurality of peptides comprises or consists of peptides selected from the group consisting of: SEQ ID NO: 136 to 185, wherein the contiguous E6 and E7 fragments comprised within the plurality of peptides together in total cover at least 70% of the combined E6 and E7 sequences.

In a further main aspect, the invention relates to a vaccine comprising:

a) a peptide selected from the group consisting of:

i) the peptide set forth in SEQ ID NO:136;

ii) the peptide set forth in SEQ ID NO:137; and

iii) the peptide set forth in SEQ ID NO:138,

and
b) a combination of peptides selected from the group consisting of:

i) the peptides set forth in SEQ ID NO:139, 140, 141 and 142;

ii) the peptides set forth in SEQ ID NO:139, 140, 143 and 144;

iii) the peptides set forth in SEQ ID NO:141, 145, 146 and 147;

iv) the peptides set forth in SEQ ID NO:145, 148, 149 and 150;

v) the peptides set forth in SEQ ID NO:151, 152, 153, 154 and 155; and

vi) the peptides set forth in SEQ ID NO:142, 156, 157, 158 and 159,

and
c) a combination of peptides selected from the group consisting of:

i) the peptides set forth in SEQ ID NO:160 and 161;

ii) the peptides set forth in SEQ ID NO:162 and 163;

iii) the peptides set forth in SEQ ID NO:164 and 165;

iv) the peptides set forth in SEQ ID NO:160 and 162; and

v) the peptides set forth in SEQ ID NO:161 and 163,

d) a combination of peptides selected from the group consisting of:

i) the peptides set forth in SEQ ID NO:166 and 167;

ii) the peptides set forth in SEQ ID NO:167, 168 and 169;

iii) the peptides set forth in SEQ ID NO:167 and 170;

iv) the peptides set forth in SEQ ID NO:171, 172 and 173; and

v) the peptides set forth in SEQ ID NO:174, 175 and 176,

and
e) a combination of peptides selected from the group consisting of:

i) the peptides set forth in SEQ ID NO:177 and 178;

ii) the peptides set forth in SEQ ID NO:179 and 180;

iii) the peptides set forth in SEQ ID NO:181 and 182;

iv) the peptides set forth in SEQ ID NO:183 and 184,

optionally, the vaccine further comprises the peptide set forth in SEQ ID NO:185.

In a further main aspect, the invention relates to a vaccine comprising a plurality of peptides, wherein said plurality of peptides comprises or consists of peptides selected from the group consisting of: SEQ ID NO: 188 to 220, wherein the contiguous E6 and E7 fragments comprised within the plurality of peptides together in total cover at least 70% of the combined E6 and E7 sequences.

In a further main aspect, the invention relates to a vaccine comprising:

a) a peptide selected from the group consisting of:

i) the peptide set forth in SEQ ID NO:188;

ii) the peptide set forth in SEQ ID NO:189; and

iii) the peptide set forth in SEQ ID NO:190,

and
b) a combination of peptides selected from the group consisting of:

i) the peptides set forth in SEQ ID NO:191, 192, 193, 194, 195 and 196;

ii) the peptides set forth in SEQ ID NO:193, 194, 197, 198, 199 and 200; and

iii) the peptides set forth in SEQ ID NO:195, 196, 201, 202, 203 and 204,

and
c) a combination of peptides selected from the group consisting of:

i) the peptides set forth in SEQ ID NO:205 and 206;

ii) the peptides set forth in SEQ ID NO:206 and 207; and

iii) the peptides set forth in SEQ ID NO:206 and 208,

and
d) a combination of peptides selected from the group consisting of:

i) the peptides set forth in SEQ ID NO:209, 210 and 211;

ii) the peptides set forth in SEQ ID NO:210, 212 and 213; and

iii) the peptides set forth in SEQ ID NO:211, 214 and 215,

and
e) a combination of peptides selected from the group consisting of:

i) the peptides set forth in SEQ ID NO:216 and 217;

ii) the peptides set forth in SEQ ID NO:218 and 219; and

iii) the peptides set forth in SEQ ID NO:219 and 220.

In a further main aspect, the invention relates to a vaccine comprising a plurality of peptides, wherein said plurality of peptides comprises or consists of peptides selected from the group consisting of: SEQ ID NO: 223 to 250, wherein the contiguous E6 and E7 fragments comprised within the plurality of peptides together in total cover at least 70% of the combined E6 and E7 sequences.

In a further main aspect, the invention relates to a vaccine comprising:

a) a combination of peptides selected from the group consisting of:

i) the peptides set forth in SEQ ID NO:223 and 224;

ii) the peptides set forth in SEQ ID NO:225 and 226; and

iii) the peptides set forth in SEQ ID NO:224 and 226,

and
b) a combination of peptides selected from the group consisting of:

i) the peptides set forth in SEQ ID NO:228, 229 and 230;

ii) the peptides set forth in SEQ ID NO:231 and 232; and

iii) the peptides set forth in SEQ ID NO:229, 230 and 233,

and
c) a combination of peptides selected from the group consisting of:

i) the peptides set forth in SEQ ID NO:234 and 235;

ii) the peptides set forth in SEQ ID NO:234 and 236; and

iii) the peptides set forth in SEQ ID NO:235 and 237,

and
d) a combination of peptides selected from the group consisting of:

i) the peptides set forth in SEQ ID NO:238, 239 and 240;

ii) the peptides set forth in SEQ ID NO:241 and 242; and

iii) the peptides set forth in SEQ ID NO:239, 240 and 243,

and optionally a peptide or combination of peptides selected from the group consisting of:

i) the peptides set forth in SEQ ID NO:244 and 245;

ii) the peptide set forth in SEQ ID NO:246;

iii) the peptide set forth in SEQ ID NO:247;

iv) the peptides set forth in SEQ ID NO:248 and 249; and

v) the peptide set forth in SEQ ID NO:250,

optionally, the vaccine further comprises the peptide set forth in SEQ ID NO:227.

In one embodiment, the vaccine is a kit comprising two or more parts, e.g. two or more vials, wherein the peptides are distributed over said two or more parts, e.g. distributed over two or more vials. For example, the peptides may be distributed over two or more immunogenic compositions. The distribution of the plurality of peptides over multiple compositions may be independent of which protein (E6 or E7) or protein region (e.g. 1-34 or 43-140) the peptides correspond to. For example, two SLPs that together cover a particular protein region can be contained within different immunogenic compositions.

Embodiments of such immunogenic compositions are described herein below. In such an embodiment, the compositions may be mixed before administration of the vaccine to the patient or the compositions may be administered separately. In one embodiment, the vaccine comprises two or more compositions comprising dried or lyophilized peptides and the vaccine further comprises a reconstitution solution and optionally an adjuvant, wherein the adjuvant may be comprised within the reconstitution solution or be provided in a further separate vial.

In one embodiment, the vaccine is for use in the treatment of cancer, such as cervical cancer or head-and-neck cancer.

In further embodiments, the invention provides (i) a vaccine comprising a plurality of peptides, or (ii) a method for treating an infection, disorder or disease caused by HPV-18, comprising administering to a human subject a plurality of peptides,

wherein said plurality of peptides comprises a set of peptides selected from the following sets:
1. the peptides set forth in SEQ ID NO:1, 4, 5, 6, 7 and 31,
2. the peptide set forth in SEQ ID NO:2, 4, 5, 6, 7 and 31,
3. the peptide set forth in SEQ ID NO:3, 4, 5, 6, 7 and 31,
4. the peptides set forth in SEQ ID NO:1, 8, 9, 10, 11, 12, 13 and 31,
5. the peptide set forth in SEQ ID NO:2, 8, 9, 10, 11, 12, 13 and 31,
6. the peptide set forth in SEQ ID NO:3, 8, 9, 10, 11, 12, 13 and 31,
7. the peptides set forth in SEQ ID NO:1, 14, 15, 16, 17, 18, 19 and 31,
8. the peptide set forth in SEQ ID NO:2, 14, 15, 16, 17, 18, 19 and 31,
9. the peptide set forth in SEQ ID NO:3, 14, 15, 16, 17, 18, 19 and 31,
10. the peptides set forth in SEQ ID NO:1, 20, 21, 22, 23, 24, 25 and 31,
11. the peptide set forth in SEQ ID NO:2, 20, 21, 22, 23, 24, 25 and 31,
12. the peptide set forth in SEQ ID NO:3, 20, 21, 22, 23, 24, 25 and 31,
13. the peptides set forth in SEQ ID NO:1, 26, 27, 28, 29, 30 and 31,
14. the peptide set forth in SEQ ID NO:2, 26, 27, 28, 29, 30 and 31,
15. the peptide set forth in SEQ ID NO:3, 26, 27, 28, 29, 30 and 31,
16. the peptides set forth in SEQ ID NO:1, 4, 5, 6, 7 and 32,
17. the peptide set forth in SEQ ID NO:2, 4, 5, 6, 7 and 32,
18. the peptide set forth in SEQ ID NO:3, 4, 5, 6, 7 and 32,
19. the peptides set forth in SEQ ID NO:1, 8, 9, 10, 11, 12, 13 and 32,
20. the peptide set forth in SEQ ID NO:2, 8, 9, 10, 11, 12, 13 and 32,
21. the peptide set forth in SEQ ID NO:3, 8, 9, 10, 11, 12, 13 and 32,
22. the peptides set forth in SEQ ID NO:1, 14, 15, 16, 17, 18, 19 and 32,
23. the peptide set forth in SEQ ID NO:2, 14, 15, 16, 17, 18, 19 and 32,
24. the peptide set forth in SEQ ID NO:3, 14, 15, 16, 17, 18, 19 and 32,
25. the peptides set forth in SEQ ID NO:1, 20, 21, 22, 23, 24, 25 and 32,
26. the peptide set forth in SEQ ID NO:2, 20, 21, 22, 23, 24, 25 and 32,
27. the peptide set forth in SEQ ID NO:3, 20, 21, 22, 23, 24, 25 and 32,
28. the peptides set forth in SEQ ID NO:1, 26, 27, 28, 29, 30 and 32,
29. the peptide set forth in SEQ ID NO:2, 26, 27, 28, 29, 30 and 32,
30. the peptide set forth in SEQ ID NO:3, 26, 27, 28, 29, 30 and 32,
31. the peptides set forth in SEQ ID NO:1, 26, 131, 28, 29 and 30,
32. the peptides set forth in SEQ ID NO:2, 26, 131, 28, 29 and 30,
33. the peptides set forth in SEQ ID NO:3, 26, 131, 28, 29 and 30.
34. the peptides set forth in SEQ ID NO:33, 34, 35 and 48,
35. the peptides set forth in SEQ ID NO:36, 37, 38 and 48,
36. the peptides set forth in SEQ ID NO:39, 40, 41 and 48,
37. the peptides set forth in SEQ ID NO:42, 43, 44 and 48,
38. the peptides set forth in SEQ ID NO:45, 46, 47 and 48,
39. the peptides set forth in SEQ ID NO:33, 34, 35, 49 and 50,
40. the peptides set forth in SEQ ID NO:36, 37, 38, 49 and 50,
41. the peptides set forth in SEQ ID NO:39, 40, 41, 49 and 50,
42. the peptides set forth in SEQ ID NO:42, 43, 44, 49 and 50,
43. the peptides set forth in SEQ ID NO:45, 46, 47, 49 and 50,
44. the peptides set forth in SEQ ID NO:33, 34, 35, 51 and 52,
45. the peptides set forth in SEQ ID NO:36, 37, 38, 51 and 52,
46. the peptides set forth in SEQ ID NO:39, 40, 41, 51 and 52,
47. the peptides set forth in SEQ ID NO:42, 43, 44, 51 and 52,
48. the peptides set forth in SEQ ID NO:45, 46, 47, 51 and 52,
49. the peptides set forth in SEQ ID NO:33, 34, 35, 53 and 54,
50. the peptides set forth in SEQ ID NO:36, 37, 38, 53 and 54,
51. the peptides set forth in SEQ ID NO:39, 40, 41, 53 and 54,
52. the peptides set forth in SEQ ID NO:42, 43, 44, 53 and 54,
53. the peptides set forth in SEQ ID NO:45, 46, 47, 53 and 54,
54. the peptides set forth in SEQ ID NO:33, 34, 35, 55 and 56,
55. the peptides set forth in SEQ ID NO:36, 37, 38, 55 and 56,
56. the peptides set forth in SEQ ID NO:39, 40, 41, 55 and 56,
57. the peptides set forth in SEQ ID NO:42, 43, 44, 55 and 56,
58. the peptides set forth in SEQ ID NO:45, 46, 47, 55 and 56,
59. the peptides set forth in SEQ ID NO:33, 34, 35, 57 and 58,
60. the peptides set forth in SEQ ID NO:36, 37, 38, 57 and 58,
61. the peptides set forth in SEQ ID NO:39, 40, 41, 57 and 58,
62. the peptides set forth in SEQ ID NO:42, 43, 44, 57 and 58,
63. the peptides set forth in SEQ ID NO:45, 46, 47, 57 and 58,
64. the peptides set forth in SEQ ID NO:33, 34, 35, 59 and 60,
65. the peptides set forth in SEQ ID NO:36, 37, 38, 59 and 60,
66. the peptides set forth in SEQ ID NO:39, 40, 41, 59 and 60,
67. the peptides set forth in SEQ ID NO:42, 43, 44, 59 and 60,
68. the peptides set forth in SEQ ID NO:45, 46, 47, 59 and 60,
69. the peptides set forth in SEQ ID NO:33, 34, 35, 61, 62 and 63,
70. the peptides set forth in SEQ ID NO:36, 37, 38, 61, 62 and 63,
71. the peptides set forth in SEQ ID NO:39, 40, 41, 61, 62 and 63,
72. the peptides set forth in SEQ ID NO:42, 43, 44, 61, 62 and 63,
73. the peptides set forth in SEQ ID NO:45, 46, 47, 61, 62 and 63,
170. the peptides set forth in SEQ ID NO:1, 26, 131, 28, 29, 30 and 31,
171. the peptides set forth in SEQ ID NO:2, 26, 131, 28, 29, 30 and 31,
172. the peptides set forth in SEQ ID NO:3, 26, 131, 28, 29, 30 and 31,
173. the peptides set forth in SEQ ID NO:1, 26, 131, 28, 29, 30 and 32,
174. the peptides set forth in SEQ ID NO:2, 26, 131, 28, 29, 30 and 32,
175. the peptides set forth in SEQ ID NO:3, 26, 131, 28, 29, 30 and 32,
176. the peptides set forth in SEQ ID NO:1, 130, 26, 131, 28, 29, 30 and 31,
177. the peptides set forth in SEQ ID NO:2, 130, 26, 131, 28, 29, 30 and 31,
178. the peptides set forth in SEQ ID NO:3, 130, 26, 131, 28, 29, 30 and 31,
179. the peptides set forth in SEQ ID NO:1, 130, 26, 131, 28, 29, 30 and 32,
180. the peptides set forth in SEQ ID NO:2, 130, 26, 131, 28, 29, 30 and 32,
181. the peptides set forth in SEQ ID NO:3, 130, 26, 131, 28, 29, 30 and 32.

In further embodiments, the invention provides (i) a vaccine comprising a plurality of peptides, or (ii) a method for treating an infection, disorder or disease caused by HPV-18, comprising administering to a human subject a plurality of peptides,

wherein said plurality of peptides comprises a set of peptides selected from the following combinations of sets:
Set 1+set 34; set 1+set 35; set 1+set 36; set 1+set 37; set 1+set 38; set 1+set 39; set 1+set 40; set 1+set 41; set 1+set 42; set 1+set 43; set 1+set 44; set 1+set 45; set 1+set 46; set 1+set 47; set 1+set 48; set 1+set 49; set 1+set 50; set 1+set 51; set 1+set 52; set 1+set 53; set 1+set 54; set 1+set 55; set 1+set 56; set 1+set 57; set 1+set 58; set 1+set 59; set 1+set 60; set 1+set 61; set 1+set 62; set 1+set 63; set 1+set 64; set 1+set 65; set 1+set 66; set 1+set 67; set 1+set 68; set 1+set 69; set 1+set 70; set 1+set 71, set 1+set 72; set 1+set 73;
set 2+set 34; set 2+set 35; set 2+set 36; set 2+set 37; set 2+set 38; set 2+set 39; set 2+set 40; set 2+set 41; set 2+set 42; set 2+set 43; set 2+set 44; set 2+set 45; set 2+set 46; set 2+set 47; set 2+set 48; set 2+set 49; set 2+set 50; set 2+set 51; set 2+set 52; set 2+set 53; set 2+set 54; set 2+set 55; set 2+set 56; set 2+set 57; set 2+set 58; set 2+set 59; set 2+set 60; set 2+set 61; set 2+set 62; set 2+set 63; set 2+set 64; set 2+set 65; set 2+set 66; set 2+set 67; set 2+set 68; set 2+set 69; set 2+set 70; set 2+set 71, set 2+set 72; set 2+set 73;
Set 3+set 34; set 3+set 35; set 3+set 36; set 3+set 37; set 3+set 38; set 3+set 39; set 3+set 40; set 3+set 41; set 3+set 42; set 3+set 43; set 3+set 44; set 3+set 45; set 3+set 46; set 3+set 47; set 3+set 48; set 3+set 49; set 3+set 50; set 3+set 51; set 3+set 52; set 3+set 53; set 3+set 54; set 3+set 55; set 3+set 56; set 3+set 57; set 3+set 58; set 3+set 59; set 3+set 60; set 3+set 61; set 3+set 62; set 3+set 63; set 3+set 64; set 3+set 65; set 3+set 66; set 3+set 67; set 3+set 68; set 3+set 69; set 3+set 70; set 3+set 71, set 3+set 72; set 3+set 73;
Set 4+set 34; set 4+set 35; set 4+set 36; set 4+set 37; set 4+set 38; set 4+set 39; set 4+set 40; set 4+set 41; set 4+set 42; set 4+set 43; set 4+set 44; set 4+set 45; set 4+set 46; set 4+set 47; set 4+set 48; set 4+set 49; set 4+set 50; set 4+set 51; set 4+set 52; set 4+set 53; set 4+set 54; set 4+set 55; set 4+set 56; set 4+set 57; set 4+set 58; set 4+set 59; set 4+set 60; set 4+set 61; set 4+set 62; set 4+set 63; set 4+set 64; set 4+set 65; set 4+set 66; set 4+set 67; set 4+set 68; set 4+set 69; set 4+set 70; set 4+set 71, set 4+set 72; set 4+set 73;
Set 5+set 34; set 5+set 35; set 5+set 36; set 5+set 37; set 5+set 38; set 5+set 39; set 5+set 40; set 5+set 41; set 5+set 42; set 5+set 43; set 5+set 44; set 5+set 45; set 5+set 46; set 5+set 47; set 5+set 48; set 5+set 49; set 5+set 50; set 5+set 51; set 5+set 52; set 5+set 53; set 5+set 54; set 5+set 55; set 5+set 56; set 5+set 57; set 5+set 58; set 5+set 59; set 5+set 60; set 5+set 61; set 5+set 62; set 5+set 63; set 5+set 64; set 5+set 65; set 5+set 66; set 5+set 67; set 5+set 68; set 5+set 69; set 5+set 70; set 5+set 71, set 5+set 72; set 5+set 73;
Set 6+set 34; set 6+set 35; set 6+set 36; set 6+set 37; set 6+set 38; set 6+set 39; set 6+set 40; set 6+set 41; set 6+set 42; set 6+set 43; set 6+set 44; set 6+set 45; set 6+set 46; set 6+set 47; set 6+set 48; set 6+set 49; set 6+set 50; set 6+set 51; set 6+set 52; set 6+set 53; set 6+set 54; set 6+set 55; set 6+set 56; set 6+set 57; set 6+set 58; set 6+set 59; set 6+set 60; set 6+set 61; set 6+set 62; set 6+set 63; set 6+set 64; set 6+set 65; set 6+set 66; set 6+set 67; set 6+set 68; set 6+set 69; set 6+set 70; set 6+set 71, set 6+set 72; set 6+set 73;
Set 7+set 34; set 7+set 35; set 7+set 36; set 7+set 37; set 7+set 38; set 7+set 39; set 7+set 40; set 7+set 41; set 7+set 42; set 7+set 43; set 7+set 44; set 7+set 45; set 7+set 46; set 7+set 47; set 7+set 48; set 7+set 49; set 7+set 50; set 7+set 51; set 7+set 52; set 7+set 53; set 7+set 54; set 7+set 55; set 7+set 56; set 7+set 57; set 7+set 58; set 7+set 59; set 7+set 60; set 7+set 61; set 7+set 62; set 7+set 63; set 7+set 64; set 7+set 65; set 7+set 66; set 7+set 67; set 7+set 68; set 7+set 69; set 7+set 70; set 7+set 71, set 7+set 72; set 7+set 73;
Set 8+set 34; set 8+set 35; set 8+set 36; set 8+set 37; set 8+set 38; set 8+set 39; set 8+set 40; set 8+set 41; set 8+set 42; set 8+set 43; set 8+set 44; set 8+set 45; set 8+set 46; set 8+set 47; set 8+set 48; set 8+set 49; set 8+set 50; set 8+set 51; set 8+set 52; set 8+set 53; set 8+set 54; set 8+set 55; set 8+set 56; set 8+set 57; set 8+set 58; set 8+set 59; set 8+set 60; set 8+set 61; set 8+set 62; set 8+set 63; set 8+set 64; set 8+set 65; set 8+set 66; set 8+set 67; set 8+set 68; set 8+set 69; set 8+set 70; set 8+set 71, set 8+set 72; set 8+set 73;
Set 9+set 34; set 9+set 35; set 9+set 36; set 9+set 37; set 9+set 38; set 9+set 39; set 9+set 40; set 9+set 41; set 9+set 42; set 9+set 43; set 9+set 44; set 9+set 45; set 9+set 46; set 9+set 47; set 9+set 48; set 9+set 49; set 9+set 50; set 9+set 51; set 9+set 52; set 9+set 53; set 9+set 54; set 9+set 55; set 9+set 56; set 9+set 57; set 9+set 58; set 9+set 59; set 9+set 60; set 9+set 61; set 9+set 62; set 9+set 63; set 9+set 64; set 9+set 65; set 9+set 66; set 9+set 67; set 9+set 68; set 9+set 69; set 9+set 70; set 9+set 71, set 9+set 72; set 9+set 73;
Set 10+set 34; set 10+set 35; set 10+set 36; set 10+set 37; set 10+set 38; set 10+set 39; set 10+set 40; set 10+set 41; set 10+set 42; set 10+set 43; set 10+set 44; set 10+set 45; set 10+set 46; set 10+set 47; set 10+set 48; set 10+set 49; set 10+set 50; set 10+set 51; set 10+set 52; set 10+set 53; set 10+set 54; set 10+set 55; set 10+set 56; set 10+set 57; set 10+set 58; set 10+set 59; set 10+set 60; set 10+set 61; set 10+set 62; set 10+set 63; set 10+set 64; set 10+set 65; set 10+set 66; set 10+set 67; set 10+set 68; set 10+set 69; set 10+set 70; set 10+set 71, set 10+set 72; set 10+set 73;
Set 11+set 34; set 11+set 35; set 11+set 36; set 11+set 37; set 11+set 38; set 11+set 39; set 11+set 40; set 11+set 41; set 11+set 42; set 11+set 43; set 11+set 44; set 11+set 45; set 11+set 46; set 11+set 47; set 11+set 48; set 11+set 49; set 11+set 50; set 11+set 51; set 11+set 52; set 11+set 53; set 11+set 54; set 11+set 55; set 11+set 56; set 11+set 57; set 11+set 58; set 11+set 59; set 11+set 60; set 11+set 61; set 11+set 62; set 11+set 63; set 11+set 64; set 11+set 65; set 11+set 66; set 11+set 67; set 11+set 68; set 11+set 69; set 11+set 70; set 11+set 71, set 11+set 72; set 11+set 73;
Set 12+set 34; set 12+set 35; set 12+set 36; set 12+set 37; set 12+set 38; set 12+set 39; set 12+set 40; set 12+set 41; set 12+set 42; set 12+set 43; set 12+set 44; set 12+set 45; set 12+set 46; set 12+set 47; set 12+set 48; set 12+set 49; set 12+set 50; set 12+set 51; set 12+set 52; set 12+set 53; set 12+set 54; set 12+set 55; set 12+set 56; set 12+set 57; set 12+set 58; set 12+set 59; set 12+set 60; set 12+set 61; set 12+set 62; set 12+set 63; set 12+set 64; set 12+set 65; set 12+set 66; set 12+set 67; set 12+set 68; set 12+set 69; set 12+set 70; set 12+set 71, set 12+set 72; set 12+set 73;
Set 13+set 34; set 13+set 35; set 13+set 36; set 13+set 37; set 13+set 38; set 13+set 39; set 13+set 40; set 13+set 41; set 13+set 42; set 13+set 43; set 13+set 44; set 13+set 45; set 13+set 46; set 13+set 47; set 13+set 48; set 13+set 49; set 13+set 50; set 13+set 51; set 13+set 52; set 13+set 53; set 13+set 54; set 13+set 55; set 13+set 56; set 13+set 57; set 13+set 58; set 13+set 59; set 13+set 60; set 13+set 61; set 13+set 62; set 13+set 63; set 13+set 64; set 13+set 65; set 13+set 66; set 13+set 67; set 13+set 68; set 13+set 69; set 13+set 70; set 13+set 71, set 13+set 72; set 13+set 73;
Set 14+set 34; set 14+set 35; set 114+set 36; set 14+set 37; set 14+set 38; set 14+set 39; set 14+set 40; set 14+set 41; set 14+set 42; set 14+set 43; set 14+set 44; set 14+set 45; set 14+set 46; set 14+set 47; set 14+set 48; set 14+set 49; set 14+set 50; set 14+set 51; set 14+set 52; set 14+set 53; set 14+set 54; set 14+set 55; set 14+set 56; set 14+set 57; set 14+set 58; set 14+set 59; set 14+set 60; set 14+set 61; set 14+set 62; set 14+set 63; set 14+set 64; set 14+set 65; set 14+set 66; set 14+set 67; set 14+set 68; set 14+set 69; set 14+set 70; set 14+set 71, set 14+set 72; set 14+set 73;
Set 15+set 34; set 15+set 35; set 15+set 36; set 15+set 37; set 15+set 38; set 15+set 39; set 15+set 40; set 15+set 41; set 15+set 42; set 15+set 43; set 15+set 44; set 15+set 45; set 15+set 46; set 15+set 47; set 15+set 48; set 15+set 49; set 15+set 50; set 15+set 51; set 15+set 52; set 15+set 53; set 15+set 54; set 15+set 55; set 15+set 56; set 15+set 57; set 15+set 58; set 15+set 59; set 15+set 60; set 15+set 61; set 15+set 62; set 15+set 63; set 15+set 64; set 15+set 65; set 15+set 66; set 15+set 67; set 15+set 68; set 15+set 69; set 15+set 70; set 15+set 71, set 15+set 72; set 15+set 73
Set 16+set 34; set 16+set 35; set 16+set 36; set 16+set 37; set 16+set 38; set 16+set 39; set 16+set 40; set 16+set 41; set 16+set 42; set 16+set 43; set 16+set 44; set 16+set 45; set 16+set 46; set 16+set 47; set 16+set 48; set 16+set 49; set 16+set 50; set 16+set 51; set 16+set 52; set 16+set 53; set 16+set 54; set 16+set 55; set 16+set 56; set 16+set 57; set 16+set 58; set 16+set 59; set 16+set 60; set 16+set 61; set 16+set 62; set 16+set 63; set 16+set 64; set 16+set 65; set 16+set 66; set 16+set 67; set 16+set 68; set 16+set 69; set 16+set 70; set 16+set 71, set 16+set 72; set 16+set 73;
Set 17+set 34; set 17+set 35; set 17+set 36; set 17+set 37; set 17+set 38; set 17+set 39; set 17+set 40; set 17+set 41; set 17+set 42; set 17+set 43; set 17+set 44; set 17+set 45; set 17+set 46; set 17+set 47; set 17+set 48; set 17+set 49; set 17+set 50; set 17+set 51; set 17+set 52; set 17+set 53; set 17+set 54; set 17+set 55; set 17+set 56; set 17+set 57; set 17+set 58; set 17+set 59; set 17+set 60; set 17+set 61; set 17+set 62; set 17+set 63; set 17+set 64; set 17+set 65; set 17+set 66; set 17+set 67; set 17+set 68; set 17+set 69; set 17+set 70; set 17+set 71, set 17+set 72; set 17+set 73;
Set 18+set 34; set 18+set 35; set 18+set 36; set 18+set 37; set 18+set 38; set 18+set 39; set 18+set 40; set 18+set 41; set 18+set 42; set 18+set 43; set 18+set 44; set 18+set 45; set 18+set 46; set 18+set 47; set 18+set 48; set 18+set 49; set 18+set 50; set 18+set 51; set 18+set 52; set 18+set 53; set 18+set 54; set 18+set 55; set 18+set 56; set 18+set 57; set 18+set 58; set 18+set 59; set 18+set 60; set 18+set 61; set 18+set 62; set 18+set 63; set 18+set 64; set 18+set 65; set 18+set 66; set 18+set 67; set 18+set 68; set 18+set 69; set 18+set 70; set 18+set 71, set 18+set 72; set 18+set 73;
Set 19+set 34; set 19+set 35; set 19+set 36; set 19+set 37; set 19+set 38; set 19+set 39; set 19+set 40; set 19+set 41; set 19+set 42; set 19+set 43; set 19+set 44; set 19+set 45; set 19+set 46; set 19+set 47; set 19+set 48; set 19+set 49; set 19+set 50; set 19+set 51; set 19+set 52; set 19+set 53; set 19+set 54; set 19+set 55; set 19+set 56; set 19+set 57; set 19+set 58; set 19+set 59; set 19+set 60; set 19+set 61; set 19+set 62; set 19+set 63; set 19+set 64; set 19+set 65; set 19+set 66; set 19+set 67; set 19+set 68; set 19+set 69; set 19+set 70; set 19+set 71, set 19+set 72; set 19+set 73;
Set 20+set 34; set 20+set 35; set 20+set 36; set 20+set 37; set 20+set 38; set 20+set 39; set 20+set 40; set 20+set 41; set 20+set 42; set 20+set 43; set 20+set 44; set 20+set 45; set 20+set 46; set 20+set 47; set 20+set 48; set 20+set 49; set 20+set 50; set 20+set 51; set 20+set 52; set 20+set 53; set 20+set 54; set 20+set 55; set 20+set 56; set 20+set 57; set 20+set 58; set 20+set 59; set 20+set 60; set 20+set 61; set 20+set 62; set 20+set 63; set 20+set 64; set 20+set 65; set 20+set 66; set 20+set 67; set 20+set 68; set 20+set 69; set 20+set 70; set 20+set 71, set 20+set 72; set 20+set 73;
Set 21+set 34; set 21+set 35; set 21+set 36; set 21+set 37; set 21+set 38; set 21+set 39; set 21+set 40; set 21+set 41; set 21+set 42; set 21+set 43; set 21+set 44; set 21+set 45; set 21+set 46; set 21+set 47; set 21+set 48; set 21+set 49; set 21+set 50; set 21+set 51; set 21+set 52; set 21+set 53; set 21+set 54; set 21+set 55; set 21+set 56; set 21+set 57; set 21+set 58; set 21+set 59; set 21+set 60; set 21+set 61; set 21+set 62; set 21+set 63; set 21+set 64; set 21+set 65; set 21+set 66; set 21+set 67; set 21+set 68; set 21+set 69; set 21+set 70; set 21+set 71, set 21+set 72; set 21+set 73;
Set 22+set 34; set 22+set 35; set 22+set 36; set 22+set 37; set 22+set 38; set 22+set 39; set 22+set 40; set 22+set 41; set 22+set 42; set 22+set 43; set 22+set 44; set 22+set 45; set 22+set 46; set 22+set 47; set 22+set 48; set 22+set 49; set 22+set 50; set 22+set 51; set 22+set 52; set 22+set 53; set 22+set 54; set 22+set 55; set 22+set 56; set 22+set 57; set 22+set 58; set 22+set 59; set 22+set 60; set 22+set 61; set 22+set 62; set 22+set 63; set 22+set 64; set 22+set 65; set 22+set 66; set 22+set 67; set 22+set 68; set 22+set 69; set 22+set 70; set 22+set 71, set 22+set 72; set 22+set 73;
Set 23+set 34; set 23+set 35; set 23+set 36; set 23+set 37; set 23+set 38; set 23+set 39; set 23+set 40; set 23+set 41; set 23+set 42; set 23+set 43; set 23+set 44; set 23+set 45; set 23+set 46; set 23+set 47; set 23+set 48; set 23+set 49; set 23+set 50; set 23+set 51; set 23+set 52; set 23+set 53; set 23+set 54; set 23+set 55; set 23+set 56; set 23+set 57; set 23+set 58; set 23+set 59; set 23+set 60; set 23+set 61; set 23+set 62; set 23+set 63; set 23+set 64; set 23+set 65; set 23+set 66; set 23+set 67; set 23+set 68; set 23+set 69; set 23+set 70; set 23+set 71, set 23+set 72; set 23+set 73;
Set 24+set 34; set 24+set 35; set 24+set 36; set 24+set 37; set 24+set 38; set 24+set 39; set 24+set 40; set 24+set 41; set 24+set 42; set 24+set 43; set 24+set 44; set 24+set 45; set 24+set 46; set 24+set 47; set 24+set 48; set 24+set 49; set 24+set 50; set 24+set 51; set 24+set 52; set 24+set 53; set 24+set 54; set 24+set 55; set 24+set 56; set 24+set 57; set 24+set 58; set 24+set 59; set 24+set 60; set 24+set 61; set 24+set 62; set 24+set 63; set 24+set 64; set 24+set 65; set 24+set 66; set 24+set 67; set 24+set 68; set 24+set 69; set 24+set 70; set 24+set 71, set 24+set 72; set 24+set 73;
Set 25+set 34; set 25+set 35; set 25+set 36; set 25+set 37; set 25+set 38; set 25+set 39; set 25+set 40; set 25+set 41; set 25+set 42; set 25+set 43; set 25+set 44; set 25+set 45; set 25+set 46; set 25+set 47; set 25+set 48; set 25+set 49; set 25+set 50; set 25+set 51; set 25+set 52; set 25+set 53; set 25+set 54; set 25+set 55; set 25+set 56; set 25+set 57; set 25+set 58; set 25+set 59; set 25+set 60; set 25+set 61; set 25+set 62; set 25+set 63; set 25+set 64; set 25+set 65; set 25+set 66; set 25+set 67; set 25+set 68; set 25+set 69; set 25+set 70; set 25+set 71, set 25+set 72; set 25+set 73;
Set 26+set 34; set 26+set 35; set 26+set 36; set 26+set 37; set 26+set 38; set 26+set 39; set 26+set 40; set 26+set 41; set 26+set 42; set 26+set 43; set 26+set 44; set 26+set 45; set 26+set 46; set 26+set 47; set 26+set 48; set 26+set 49; set 26+set 50; set 26+set 51; set 26+set 52; set 26+set 53; set 26+set 54; set 26+set 55; set 26+set 56; set 26+set 57; set 26+set 58; set 26+set 59; set 26+set 60; set 26+set 61; set 26+set 62; set 26+set 63; set 26+set 64; set 26+set 65; set 26+set 66; set 26+set 67; set 26+set 68; set 26+set 69; set 26+set 70; set 26+set 71, set 26+set 72; set 26+set 73;
Set 27+set 34; set 27+set 35; set 27+set 36; set 27+set 37; set 27+set 38; set 27+set 39; set 27+set 40; set 27+set 41; set 27+set 42; set 27+set 43; set 27+set 44; set 27+set 45; set 27+set 46; set 27+set 47; set 27+set 48; set 27+set 49; set 27+set 50; set 27+set 51; set 27+set 52; set 27+set 53; set 27+set 54; set 27+set 55; set 27+set 56; set 27+set 57; set 27+set 58; set 27+set 59; set 27+set 60; set 27+set 61; set 27+set 62; set 27+set 63; set 27+set 64; set 27+set 65; set 27+set 66; set 27+set 67; set 27+set 68; set 27+set 69; set 27+set 70; set 27+set 71, set 27+set 72; set 27+set 73;
Set 28+set 34; set 28+set 35; set 28+set 36; set 28+set 37; set 28+set 38; set 28+set 39; set 28+set 40; set 28+set 41; set 28+set 42; set 28+set 43; set 28+set 44; set 28+set 45; set 28+set 46; set 28+set 47; set 28+set 48; set 28+set 49; set 28+set 50; set 28+set 51; set 28+set 52; set 28+set 53; set 28+set 54; set 28+set 55; set 28+set 56; set 28+set 57; set 28+set 58; set 28+set 59; set 28+set 60; set 28+set 61; set 28+set 62; set 28+set 63; set 28+set 64; set 28+set 65; set 28+set 66; set 28+set 67; set 28+set 68; set 28+set 69; set 28+set 70; set 28+set 71, set 28+set 72; set 28+set 73;
Set 29+set 34; set 29+set 35; set 29+set 36; set 29+set 37; set 29+set 38; set 29+set 39; set 29+set 40; set 29+set 41; set 29+set 42; set 29+set 43; set 29+set 44; set 29+set 45; set 29+set 46; set 29+set 47; set 29+set 48; set 29+set 49; set 29+set 50; set 29+set 51; set 29+set 52; set 29+set 53; set 29+set 54; set 29+set 55; set 29+set 56; set 29+set 57; set 29+set 58; set 29+set 59; set 29+set 60; set 29+set 61; set 29+set 62; set 29+set 63; set 29+set 64; set 29+set 65; set 29+set 66; set 29+set 67; set 29+set 68; set 29+set 69; set 29+set 70; set 29+set 71, set 29+set 72; set 29+set 73;
Set 30+set 34; set 30+set 35; set 30+set 36; set 30+set 37; set 30+set 38; set 30+set 39; set 30+set 40; set 30+set 41; set 30+set 42; set 30+set 43; set 30+set 44; set 30+set 45; set 30+set 46; set 30+set 47; set 30+set 48; set 30+set 49; set 30+set 50; set 30+set 51; set 30+set 52; set 30+set 53; set 30+set 54; set 30+set 55; set 30+set 56; set 30+set 57; set 30+set 58; set 30+set 59; set 30+set 60; set 30+set 61; set 30+set 62; set 30+set 63; set 30+set 64; set 30+set 65; set 30+set 66; set 30+set 67; set 30+set 68; set 30+set 69; set 30+set 70; set 30+set 71, set 30+set 72; set 30+set 73;
Set 31+set 34; set 31+set 35; set 31+set 36; set 31+set 37; set 31+set 38; set 31+set 39; set 31+set 40; set 31+set 41; set 31+set 42; set 31+set 43; set 31+set 44; set 31+set 45; set 31+set 46; set 31+set 47; set 31+set 48; set 31+set 49; set 31+set 50; set 31+set 51; set 31+set 52; set 31+set 53; set 31+set 54; set 31+set 55; set 31+set 56; set 31+set 57; set 31+set 58; set 31+set 59; set 31+set 60; set 31+set 61; set 31+set 62; set 31+set 63; set 31+set 64; set 31+set 65; set 31+set 66; set 31+set 67; set 31+set 68; set 31+set 69; set 31+set 70; set 31+set 71, set 31+set 72; set 31+set 73;
Set 32+set 34; set 32+set 35; set 32+set 36; set 32+set 37; set 32+set 38; set 32+set 39; set 32+set 40; set 32+set 41; set 32+set 42; set 32+set 43; set 32+set 44; set 32+set 45; set 32+set 46; set 32+set 47; set 32+set 48; set 32+set 49; set 32+set 50; set 32+set 51; set 32+set 52; set 32+set 53; set 32+set 54; set 32+set 55; set 32+set 56; set 32+set 57; set 32+set 58; set 32+set 59; set 32+set 60; set 32+set 61; set 32+set 62; set 32+set 63; set 32+set 64; set 32+set 65; set 32+set 66; set 32+set 67; set 32+set 68; set 32+set 69; set 32+set 70; set 32+set 71, set 32+set 72; set 32+set 73;
Set 33+set 34; set 33+set 35; set 33+set 36; set 33+set 37; set 33+set 38; set 33+set 39; set 33+set 40; set 33+set 41; set 33+set 42; set 33+set 43; set 33+set 44; set 33+set 45; set 33+set 46; set 33+set 47; set 33+set 48; set 33+set 49; set 33+set 50; set 33+set 51; set 33+set 52; set 33+set 53; set 33+set 54; set 33+set 55; set 33+set 56; set 33+set 57; set 33+set 58; set 33+set 59; set 33+set 60; set 33+set 61; set 33+set 62; set 33+set 63; set 33+set 64; set 33+set 65; set 33+set 66; set 33+set 67; set 33+set 68; set 33+set 69; set 33+set 70; set 33+set 71, set 33+set 72; set 33+set 73;
Set 170+set 34; set 170+set 35; set 170+set 36; set 170+set 37; set 170+set 38; set 170+set 39; set 170+set 40; set 170+set 41; set 170+set 42; set 170+set 43; set 170+set 44; set 170+set 45; set 170+set 46; set 170+set 47; set 170+set 48; set 170+set 49; set 170+set 50; set 170+set 51; set 170+set 52; set 170+set 53; set 170+set 54; set 170+set 55; set 170+set 56; set 170+set 57; set 170+set 58; set 170+set 59; set 170+set 60; set 170+set 61; set 170+set 62; set 170+set 63; set 170+set 64; set 170+set 65; set 170+set 66; set 170+set 67; set 170+set 68; set 170+set 69; set 170+set 70; set 170+set 71, set 170+set 72; set 170+set 73;
Set 171+set 34; set 171+set 35; set 171+set 36; set 171+set 37; set 171+set 38; set 171+set 39; set 171+set 40; set 171+set 41; set 171+set 42; set 171+set 43; set 171+set 44; set 171+set 45; set 171+set 46; set 171+set 47; set 171+set 48; set 171+set 49; set 171+set 50; set 171+set 51; set 171+set 52; set 171+set 53; set 171+set 54; set 171+set 55; set 171+set 56; set 171+set 57; set 171+set 58; set 171+set 59; set 171+set 60; set 171+set 61; set 171+set 62; set 171+set 63; set 171+set 64; set 171+set 65; set 171+set 66; set 171+set 67; set 171+set 68; set 171+set 69; set 171+set 70; set 171+set 71, set 171+set 72; set 171+set 73;
Set 172+set 34; set 172+set 35; set 172+set 36; set 172+set 37; set 172+set 38; set 172+set 39; set 172+set 40; set 172+set 41; set 172+set 42; set 172+set 43; set 172+set 44; set 172+set 45; set 172+set 46; set 172+set 47; set 172+set 48; set 172+set 49; set 172+set 50; set 172+set 51; set 172+set 52; set 172+set 53; set 172+set 54; set 172+set 55; set 172+set 56; set 172+set 57; set 172+set 58; set 172+set 59; set 172+set 60; set 172+set 61; set 172+set 62; set 172+set 63; set 172+set 64; set 172+set 65; set 172+set 66; set 172+set 67; set 172+set 68; set 172+set 69; set 172+set 70; set 172+set 71, set 172+set 72; set 172+set 73;
Set 173+set 34; set 173+set 35; set 173+set 36; set 173+set 37; set 173+set 38; set 173+set 39; set 173+set 40; set 173+set 41; set 173+set 42; set 173+set 43; set 173+set 44; set 173+set 45; set 173+set 46; set 173+set 47; set 173+set 48; set 173+set 49; set 173+set 50; set 173+set 51; set 173+set 52; set 173+set 53; set 173+set 54; set 173+set 55; set 173+set 56; set 173+set 57; set 173+set 58; set 173+set 59; set 173+set 60; set 173+set 61; set 173+set 62; set 173+set 63; set 173+set 64; set 173+set 65; set 173+set 66; set 173+set 67; set 173+set 68; set 173+set 69; set 173+set 70; set 173+set 71, set 173+set 72; set 173+set 73;
Set 174+set 34; set 174+set 35; set 174+set 36; set 174+set 37; set 174+set 38; set 174+set 39; set 174+set 40; set 174+set 41; set 174+set 42; set 174+set 43; set 174+set 44; set 174+set 45; set 174+set 46; set 174+set 47; set 174+set 48; set 174+set 49; set 174+set 50; set 174+set 51; set 174+set 52; set 174+set 53; set 174+set 54; set 174+set 55; set 174+set 56; set 174+set 57; set 174+set 58; set 174+set 59; set 174+set 60; set 174+set 61; set 174+set 62; set 174+set 63; set 174+set 64; set 174+set 65; set 174+set 66; set 174+set 67; set 174+set 68; set 174+set 69; set 174+set 70; set 174+set 71, set 174+set 72; set 174+set 73;
Set 175+set 34; set 175+set 35; set 175+set 36; set 175+set 37; set 175+set 38; set 175+set 39; set 175+set 40; set 175+set 41; set 175+set 42; set 175+set 43; set 175+set 44; set 175+set 45; set 175+set 46; set 175+set 47; set 175+set 48; set 175+set 49; set 175+set 50; set 175+set 51; set 175+set 52; set 175+set 53; set 175+set 54; set 175+set 55; set 175+set 56; set 175+set 57; set 175+set 58; set 175+set 59; set 175+set 60; set 175+set 61; set 175+set 62; set 175+set 63; set 175+set 64; set 175+set 65; set 175+set 66; set 175+set 67; set 175+set 68; set 175+set 69; set 175+set 70; set 175+set 71, set 175+set 72; set 175+set 73;
Set 176+set 34; set 176+set 35; set 176+set 36; set 176+set 37; set 176+set 38; set 176+set 39; set 176+set 40; set 176+set 41; set 1+set 42; set 176+set 43; set 176+set 44; set 176+set 45; set 176+set 46; set 176+set 47; set 176+set 48; set 176+set 49; set 176+set 50; set 176+set 51; set 176+set 52; set 176+set 53; set 176+set 54; set 176+set 55; set 176+set 56; set 176+set 57; set 176+set 58; set 176+set 59; set 176+set 60; set 176+set 61; set 176+set 62; set 176+set 63; set 176+set 64; set 176+set 65; set 176+set 66; set 176+set 67; set 176+set 68; set 176+set 69; set 176+set 70; set 176+set 71, set 176+set 72; set 176+set 73;
Set 177+set 34; set 177+set 35; set 177+set 36; set 177+set 37; set 177+set 38; set 177+set 39; set 177+set 40; set 177+set 41; set 177+set 42; set 177+set 43; set 177+set 44; set 177+set 45; set 177+set 46; set 177+set 47; set 177+set 48; set 177+set 49; set 177+set 50; set 177+set 51; set 177+set 52; set 177+set 53; set 177+set 54; set 177+set 55; set 177+set 56; set 177+set 57; set 177+set 58; set 177+set 59; set 177+set 60; set 177+set 61; set 177+set 62; set 177+set 63; set 177+set 64; set 177+set 65; set 177+set 66; set 177+set 67; set 177+set 68; set 177+set 69; set 177+set 70; set 177+set 71, set 177+set 72; set 177+set 73;
Set 178+set 34; set 178+set 35; set 178+set 36; set 178+set 37; set 178+set 38; set 178+set 39; set 178+set 40; set 178+set 41; set 178+set 42; set 178+set 43; set 178+set 44; set 178+set 45; set 178+set 46; set 178+set 47; set 178+set 48; set 178+set 49; set 178+set 50; set 178+set 51; set 178+set 52; set 178+set 53; set 178+set 54; set 178+set 55; set 178+set 56; set 178+set 57; set 178+set 58; set 178+set 59; set 178+set 60; set 178+set 61; set 178+set 62; set 178+set 63; set 178+set 64; set 178+set 65; set 178+set 66; set 178+set 67; set 178+set 68; set 178+set 69; set 178+set 70; set 178+set 71, set 178+set 72; set 178+set 73;
Set 179+set 34; set 179+set 35; set 179+set 36; set 179+set 37; set 179+set 38; set 179+set 39; set 179+set 40; set 179+set 41; set 179+set 42; set 179+set 43; set 179+set 44; set 179+set 45; set 179+set 46; set 179+set 47; set 179+set 48; set 179+set 49; set 179+set 50; set 179+set 51; set 179+set 52; set 179+set 53; set 179+set 54; set 179+set 55; set 179+set 56; set 179+set 57; set 179+set 58; set 179+set 59; set 179+set 60; set 179+set 61; set 179+set 62; set 179+set 63; set 179+set 64; set 179+set 65; set 179+set 66; set 179+set 67; set 179+set 68; set 179+set 69; set 179+set 70; set 179+set 71, set 179+set 72; set 179+set 73;
Set 180+set 34; set 180+set 35; set 180+set 36; set 180+set 37; set 180+set 38; set 180+set 39; set 180+set 40; set 180+set 41; set 180+set 42; set 180+set 43; set 180+set 44; set 180+set 45; set 180+set 46; set 180+set 47; set 180+set 48; set 180+set 49; set 180+set 50; set 180+set 51; set 180+set 52; set 180+set 53; set 180+set 54; set 180+set 55; set 180+set 56; set 180+set 57; set 180+set 58; set 180+set 59; set 180+set 60; set 180+set 61; set 180+set 62; set 180+set 63; set 180+set 64; set 180+set 65; set 180+set 66; set 180+set 67; set 180+set 68; set 180+set 69; set 180+set 70; set 180+set 71, set 180+set 72; set 180+set 73;
Set 181+set 34; set 181+set 35; set 181+set 36; set 181+set 37; set 181+set 38; set 181+set 39; set 181+set 40; set 181+set 41; set 181+set 42; set 181+set 43; set 181+set 44; set 181+set 45; set 181+set 46; set 181+set 47; set 181+set 48; set 181+set 49; set 181+set 50; set 181+set 51; set 181+set 52; set 181+set 53; set 181+set 54; set 181+set 55; set 181+set 56; set 181+set 57; set 181+set 58; set 181+set 59; set 181+set 60; set 181+set 61; set 181+set 62; set 181+set 63; set 181+set 64; set 181+set 65; set 181+set 66; set 181+set 67; set 181+set 68; set 181+set 69; set 181+set 70; set 181+set 71, set 181+set 72; set 181+set 73.

In further embodiments, the invention provides (i) a vaccine comprising a plurality of peptides, or (ii) a method for treating an infection, disorder or disease caused by HPV-45, comprising administering to a human subject a plurality of peptides,

wherein said plurality of peptides comprises a set of peptides selected from the following sets:
74. the peptides set forth in SEQ ID NO:64, 68, 72, 73, 74, 75 and 90,
75. the peptides set forth in SEQ ID NO:65, 68, 72, 73, 74, 75 and 90,
76. the peptides set forth in SEQ ID NO:66, 68, 72, 73, 74, 75 and 90,
77. the peptides set forth in SEQ ID NO:67, 68, 72, 73, 74, 75 and 90,
78. the peptides set forth in SEQ ID NO:64, 69, 72, 73, 74, 75 and 90,
79. the peptides set forth in SEQ ID NO:65, 69, 72, 73, 74, 75 and 90,
80. the peptides set forth in SEQ ID NO:66, 69, 72, 73, 74, 75 and 90,
81. the peptides set forth in SEQ ID NO:67, 69, 72, 73, 74, 75 and 90,
82. the peptides set forth in SEQ ID NO:64, 70, 72, 73, 74, 75 and 90,
83. the peptides set forth in SEQ ID NO:65, 70, 72, 73, 74, 75 and 90,
84. the peptides set forth in SEQ ID NO:66, 70, 72, 73, 74, 75 and 90,
85. the peptides set forth in SEQ ID NO:67, 70, 72, 73, 74, 75 and 90,
86. the peptides set forth in SEQ ID NO:64, 71, 72, 73, 74, 75 and 90,
87. the peptides set forth in SEQ ID NO:65, 71, 72, 73, 74, 75 and 90,
88. the peptides set forth in SEQ ID NO:66, 71, 72, 73, 74, 75 and 90,
89. the peptides set forth in SEQ ID NO:67, 71, 72, 73, 74, 75 and 90,
90. the peptides set forth in SEQ ID NO:64, 68, 76, 77, 78, 79, 80, 81 and 90,
91. the peptides set forth in SEQ ID NO:65, 68, 76, 77, 78, 79, 80, 81 and 90,
92. the peptides set forth in SEQ ID NO:66, 68, 76, 77, 78, 79, 80, 81 and 90,
93. the peptides set forth in SEQ ID NO:67, 68, 76, 77, 78, 79, 80, 81 and 90,
94. the peptides set forth in SEQ ID NO:64, 69, 76, 77, 78, 79, 80, 81 and 90,
95. the peptides set forth in SEQ ID NO:65, 69, 76, 77, 78, 79, 80, 81 and 90,
96. the peptides set forth in SEQ ID NO:66, 69, 76, 77, 78, 79, 80, 81 and 90,
97. the peptides set forth in SEQ ID NO:67, 69, 76, 77, 78, 79, 80, 81 and 90,
98. the peptides set forth in SEQ ID NO:64, 70, 76, 77, 78, 79, 80, 81 and 90,
99. the peptides set forth in SEQ ID NO:65, 70, 76, 77, 78, 79, 80, 81 and 90,
100. the peptides set forth in SEQ ID NO:66, 70, 76, 77, 78, 79, 80, 81 and 90,
101. the peptides set forth in SEQ ID NO:67, 70, 76, 77, 78, 79, 80, 81 and 90,
102. the peptides set forth in SEQ ID NO:64, 71, 76, 77, 78, 79, 80, 81 and 90,
103. the peptides set forth in SEQ ID NO:65, 71, 76, 77, 78, 79, 80, 81 and 90,
104. the peptides set forth in SEQ ID NO:66, 71, 76, 77, 78, 79, 80, 81 and 90,
105. the peptides set forth in SEQ ID NO:67, 71, 67, 77, 78, 79, 80, 81 and 90,
106. the peptides set forth in SEQ ID NO:64, 68, 82, 83, 84, 85 and 90,
107. the peptides set forth in SEQ ID NO:65, 68, 82, 83, 84, 85 and 90,
108. the peptides set forth in SEQ ID NO:66, 68, 82, 83, 84, 85 and 90,
109. the peptides set forth in SEQ ID NO:67, 68, 82, 83, 84, 85 and 90,
110. the peptides set forth in SEQ ID NO:64, 69, 82, 83, 84, 85 and 90,
111. the peptides set forth in SEQ ID NO:65, 69, 82, 83, 84, 85 and 90,
112. the peptides set forth in SEQ ID NO:66, 69, 82, 83, 84, 85 and 90,
113. the peptides set forth in SEQ ID NO:67, 69, 82, 83, 84, 85 and 90,
114. the peptides set forth in SEQ ID NO:64, 70, 82, 83, 84, 85 and 90,
115. the peptides set forth in SEQ ID NO:65, 70, 82, 83, 84, 85 and 90,
116. the peptides set forth in SEQ ID NO:66, 70, 82, 83, 84, 85 and 90,
117. the peptides set forth in SEQ ID NO:67, 70, 82, 83, 84, 85 and 90,
118. the peptides set forth in SEQ ID NO:64, 71, 82, 83, 84, 85 and 90,
119. the peptides set forth in SEQ ID NO:65, 71, 82, 83, 84, 85 and 90,
120. the peptides set forth in SEQ ID NO:66, 71, 82, 38, 84, 85 and 90,
121. the peptides set forth in SEQ ID NO:67, 71, 82, 83, 84, 85 and 90,
122. the peptides set forth in SEQ ID NO:64, 68, 86, 87, 88, 89 and 90,
123. the peptides set forth in SEQ ID NO:65, 68, 86, 87, 88, 89 and 90,
124. the peptides set forth in SEQ ID NO:66, 68, 86, 87, 88, 89 and 90,
125. the peptides set forth in SEQ ID NO:67, 68, 86, 87, 88, 89 and 90,
126. the peptides set forth in SEQ ID NO:64, 69, 86, 87, 88, 89 and 90,
127. the peptides set forth in SEQ ID NO:65, 69, 86, 87, 88, 89 and 90,
128. the peptides set forth in SEQ ID NO:66, 69, 86, 87, 88, 89 and 90,
129. the peptides set forth in SEQ ID NO:67, 69, 86, 87, 88, 89 and 90,
130. the peptides set forth in SEQ ID NO:64, 70, 86, 87, 88, 89 and 90,
131. the peptides set forth in SEQ ID NO:65, 70, 86, 87, 88, 89 and 90,
132. the peptides set forth in SEQ ID NO:66, 70, 86, 87, 88, 89 and 90,
133. the peptides set forth in SEQ ID NO:67, 70, 86, 87, 88, 89 and 90,
134. the peptides set forth in SEQ ID NO:64, 71, 86, 87, 88, 89 and 90,
135. the peptides set forth in SEQ ID NO:65, 71, 86, 87, 88, 89 and 90,
136. the peptides set forth in SEQ ID NO:66, 71, 86, 87, 88, 89 and 90,
137. the peptides set forth in SEQ ID NO:67, 71, 86, 87, 88, 89 and 90.
138. the peptides set forth in SEQ ID NO:91, 92, 93, 110 and 111,
139. the peptides set forth in SEQ ID NO:94, 95, 96, 110 and 111,
140. the peptides set forth in SEQ ID NO:97, 98, 99, 110 and 111,
141. the peptides set forth in SEQ ID NO:100, 101, 102, 103, 110 and 111,
142. the peptides set forth in SEQ ID NO:104, 105, 110 and 110,
143. the peptides set forth in SEQ ID NO:106, 107, 108, 109, 110 and 111,
144. the peptides set forth in SEQ ID NO: 93, 104, 132, 110 and 111,
145. the peptides set forth in SEQ ID NO: 93, 104, 133, 110 and 111,
146. the peptides set forth in SEQ ID NO:91, 92, 93, 112, 113 and 114,
147. the peptides set forth in SEQ ID NO:94, 95, 96, 112, 113 and 114,
148. the peptides set forth in SEQ ID NO:97, 98, 99, 112, 113 and 114,
149. the peptides set forth in SEQ ID NO:100, 101, 102, 103, 112, 113 and 114,
150. the peptides set forth in SEQ ID NO:104, 105, 112, 113 and 114,
151. the peptides set forth in SEQ ID NO:106, 107, 108, 109, 112, 113 and 114,
152. the peptides set forth in SEQ ID NO: 93, 104, 132, 112, 113 and 114,
153. the peptides set forth in SEQ ID NO: 93, 104, 133, 112, 113 and 114,
154. the peptides set forth in SEQ ID NO:91, 92, 93, 115, 116 and 117,
155. the peptides set forth in SEQ ID NO:94, 95, 96, 115, 116 and 117,
156. the peptides set forth in SEQ ID NO:97, 98, 99, 115, 116 and 117,
157. the peptides set forth in SEQ ID NO:100, 101, 102, 103, 115, 116 and 117,
158. the peptides set forth in SEQ ID NO:104, 105, 115, 116 and 117,
159. the peptides set forth in SEQ ID NO:106, 107, 108, 109, 115, 116 and 117,
160. the peptides set forth in SEQ ID NO: 93, 104, 132, 115, 116 and 117,
161. the peptides set forth in SEQ ID NO: 93, 104, 133, 115, 116 and 117,
162. the peptides set forth in SEQ ID NO:91, 92, 93, 118 and 119,
163. the peptides set forth in SEQ ID NO:94, 95, 96, 118 and 119,
164. the peptides set forth in SEQ ID NO:97, 98, 99, 118 and 119,
165. the peptides set forth in SEQ ID NO:100, 101, 102, 103, 118 and 119,
166. the peptides set forth in SEQ ID NO:104, 105, 118 and 119,
167. the peptides set forth in SEQ ID NO:106, 107, 108, 109, 118 and 119
168. the peptides set forth in SEQ ID NO: 93, 104, 132, 118 and 119,
169. the peptides set forth in SEQ ID NO: 93, 104, 133, 118 and 119.

In further embodiments, the invention provides (i) a vaccine comprising a plurality of peptides, or (ii) a method for treating an infection, disorder or disease caused by HPV-45, comprising administering to a human subject a plurality of peptides,

wherein said plurality of peptides comprises a set of peptides selected from the following combinations of sets:
Set 74+set 138; set 74+set 139; set 74+set 140; set 74+set 141; set 74+set 142; set 74+set 143; set 74+set 144; set 74+set 145; set 74+set 146; set 74+set 147; set 74+set 148; set 74+set 149; set 74+set 150; set 74+set 151; set 74+set 152; set 74+set 153; set 74+set 154; set 74+set 155; set 74+set 156; set 74+set 157; set 74+set 158; set 74+set 159; set 74+set 160; set 74+set 161; set 74+set 162; set 74+set 163; set 74+set 164; set 74+set 165; set 74+set 166; set 74+set 167; set 74+set 168; set 74+set 169;
Set 75+set 138; set 75+set 139; set 75+set 140; set 75+set 141; set 75+set 142; set 75+set 143; set 75+set 144; set 75+set 145; set 75+set 146; set 75+set 147; set 75+set 148; set 75+set 149; set 75+set 150; set 75+set 151; set 75+set 152; set 75+set 153; set 75+set 154; set 75+set 155; set 75+set 156; set 75+set 157; set 75+set 158; set 75+set 159; set 75+set 160; set 75+set 161; set 75+set 162; set 75+set 163; set 75+set 164; set 75+set 165; set 75+set 166; set 75+set 167; set 75+set 168; set 75+set 169;
Set 76+set 138; set 76+set 139; set 76+set 140; set 76+set 141; set 76+set 142; set 76+set 143; set 76+set 144; set 76+set 145; set 76+set 146; set 76+set 147; set 76+set 148; set 76+set 149; set 76+set 150; set 76+set 151; set 76+set 152; set 76+set 153; set 76+set 154; set 76+set 155; set 76+set 156; set 76+set 157; set 76+set 158; set 76+set 159; set 76+set 160; set 76+set 161; set 76+set 162; set 76+set 163; set 76+set 164; set 76+set 165; set 76+set 166; set 76+set 167; set 76+set 168; set 76+set 169;
Set 77+set 138; set 77+set 139; set 77+set 140; set 77+set 141; set 77+set 142; set 77+set 143; set 77+set 144; set 77+set 145; set 77+set 146; set 77+set 147; set 77+set 148; set 77+set 149; set 77+set 150; set 77+set 151; set 77+set 152; set 77+set 153; set 77+set 154; set 77+set 155; set 77+set 156; set 77+set 157; set 77+set 158; set 77+set 159; set 77+set 160; set 77+set 161; set 77+set 162; set 77+set 163; set 77+set 164; set 77+set 165; set 77+set 166; set 77+set 167; set 77+set 168; set 77+set 169;
Set 78+set 138; set 78+set 139; set 78+set 140; set 78+set 141; set 78+set 142; set 78+set 143; set 78+set 144; set 78+set 145; set 78+set 146; set 78+set 147; set 78+set 148; set 78+set 149; set 78+set 150; set 78+set 151; set 78+set 152; set 78+set 153; set 78+set 154; set 78+set 155; set 78+set 156; set 78+set 157; set 78+set 158; set 78+set 159; set 78+set 160; set 78+set 161; set 78+set 162; set 78+set 163; set 78+set 164; set 78+set 165; set 78+set 166; set 78+set 167; set 78+set 168; set 78+set 169;
Set 79+set 138; set 79+set 139; set 79+set 140; set 79+set 141; set 79+set 142; set 79+set 143; set 79+set 144; set 79+set 145; set 79+set 146; set 79+set 147; set 79+set 148; set 79+set 149; set 79+set 150; set 79+set 151; set 79+set 152; set 79+set 153; set 79+set 154; set 79+set 155; set 79+set 156; set 79+set 157; set 79+set 158; set 79+set 159; set 79+set 160; set 79+set 161; set 79+set 162; set 79+set 163; set 79+set 164; set 79+set 165; set 79+set 166; set 79+set 167; set 79+set 168; set 79+set 169;
Set 80+set 138; set 80+set 139; set 80+set 140; set 80+set 141; set 80+set 142; set 80+set 143; set 80+set 144; set 80+set 145; set 80+set 146; set 80+set 147; set 80+set 148; set 80+set 149; set 80+set 150; set 80+set 151; set 80+set 152; set 80+set 153; set 80+set 154; set 80+set 155; set 80+set 156; set 80+set 157; set 80+set 158; set 80+set 159; set 80+set 160; set 80+set 161; set 80+set 162; set 80+set 163; set 80+set 164; set 80+set 165; set 80+set 166; set 80+set 167; set 80+set 168; set 80+set 169;
Set 81+set 138; set 81+set 139; set 81+set 140; set 81+set 141; set 81+set 142; set 81+set 143; set 81+set 144; set 81+set 145; set 81+set 146; set 81+set 147; set 81+set 148; set 81+set 149; set 81+set 150; set 81+set 151; set 81+set 152; set 81+set 153; set 81+set 154; set 81+set 155; set 81+set 156; set 81+set 157; set 81+set 158; set 81+set 159; set 81+set 160; set 81+set 161; set 81+set 162; set 81+set 163; set 81+set 164; set 81+set 165; set 81+set 166; set 81+set 167; set 81+set 168; set 81+set 169;
Set 82+set 138; set 82+set 139; set 82+set 140; set 82+set 141; set 82+set 142; set 82+set 143; set 82+set 144; set 82+set 145; set 82+set 146; set 82+set 147; set 82+set 148; set 82+set 149; set 82+set 150; set 82+set 151; set 82+set 152; set 82+set 153; set 82+set 154; set 82+set 155; set 82+set 156; set 82+set 157; set 82+set 158; set 82+set 159; set 82+set 160; set 82+set 161; set 82+set 162; set 82+set 163; set 82+set 164; set 82+set 165; set 82+set 166; set 82+set 167; set 82+set 168; set 82+set 169;
Set 83+set 138; set 83+set 139; set 83+set 140; set 83+set 141; set 83+set 142; set 83+set 143; set 83+set 144; set 83+set 145; set 83+set 146; set 83+set 147; set 83+set 148; set 83+set 149; set 83+set 150; set 83+set 151; set 83+set 152; set 83+set 153; set 83+set 154; set 83+set 155; set 83+set 156; set 83+set 157; set 83+set 158; set 83+set 159; set 83+set 160; set 83+set 161; set 83+set 162; set 83+set 163; set 83+set 164; set 83+set 165; set 83+set 166; set 83+set 167; set 83+set 168; set 83+set 169;
Set 84+set 138; set 84+set 139; set 84+set 140; set 84+set 141; set 84+set 142; set 84+set 143; set 84+set 144; set 84+set 145; set 84+set 146; set 84+set 147; set 84+set 148; set 84+set 149; set 84+set 150; set 84+set 151; set 84+set 152; set 84+set 153; set 84+set 154; set 84+set 155; set 84+set 156; set 84+set 157; set 84+set 158; set 84+set 159; set 84+set 160; set 84+set 161; set 84+set 162; set 84+set 163; set 84+set 164; set 84+set 165; set 84+set 166; set 84+set 167; set 84+set 168; set 84+set 169;
Set 85+set 138; set 85+set 139; set 85+set 140; set 85+set 141; set 85+set 142; set 85+set 143; set 85+set 144; set 85+set 145; set 85+set 146; set 85+set 147; set 85+set 148; set 85+set 149; set 85+set 150; set 85+set 151; set 85+set 152; set 85+set 153; set 85+set 154; set 85+set 155; set 85+set 156; set 85+set 157; set 85+set 158; set 85+set 159; set 85+set 160; set 85+set 161; set 85+set 162; set 85+set 163; set 85+set 164; set 85+set 165; set 85+set 166; set 85+set 167; set 85+set 168; set 85+set 169;
Set 86+set 138; set 86+set 139; set 86+set 140; set 86+set 141; set 86+set 142; set 86+set 143; set 86+set 144; set 86+set 145; set 86+set 146; set 86+set 147; set 86+set 148; set 86+set 149; set 86+set 150; set 86+set 151; set 86+set 152; set 86+set 153; set 86+set 154; set 86+set 155; set 86+set 156; set 86+set 157; set 86+set 158; set 86+set 159; set 86+set 160; set 86+set 161; set 86+set 162; set 86+set 163; set 86+set 164; set 86+set 165; set 86+set 166; set 86+set 167; set 86+set 168; set 86+set 169;
Set 87+set 138; set 87+set 139; set 87+set 140; set 87+set 141; set 87+set 142; set 87+set 143; set 87+set 144; set 87+set 145; set 87+set 146; set 87+set 147; set 87+set 148; set 87+set 149; set 87+set 150; set 87+set 151; set 87+set 152; set 87+set 153; set 87+set 154; set 87+set 155; set 87+set 156; set 87+set 157; set 87+set 158; set 87+set 159; set 87+set 160; set 87+set 161; set 87+set 162; set 87+set 163; set 87+set 164; set 87+set 165; set 87+set 166; set 87+set 167; set 87+set 168; set 87+set 169;
Set 88+set 138; set 88+set 139; set 88+set 140; set 88+set 141; set 88+set 142; set 88+set 143; set 88+set 144; set 88+set 145; set 88+set 146; set 88+set 147; set 88+set 148; set 88+set 149; set 88+set 150; set 88+set 151; set 88+set 152; set 88+set 153; set 88+set 154; set 88+set 155; set 88+set 156; set 88+set 157; set 88+set 158; set 88+set 159; set 88+set 160; set 88+set 161; set 88+set 162; set 88+set 163; set 88+set 164; set 88+set 165; set 88+set 166; set 88+set 167; set 88+set 168; set 88+set 169;
Set 89+set 138; set 89+set 139; set 89+set 140; set 89+set 141; set 89+set 142; set 89+set 143; set 89+set 144; set 89+set 145; set 89+set 146; set 89+set 147; set 89+set 148; set 89+set 149; set 89+set 150; set 89+set 151; set 89+set 152; set 89+set 153; set 89+set 154; set 89+set 155; set 89+set 156; set 89+set 157; set 89+set 158; set 89+set 159; set 89+set 160; set 89+set 161; set 89+set 162; set 89+set 163; set 89+set 164; set 89+set 165; set 89+set 166; set 89+set 167; set 89+set 168; set 89+set 169;
Set 90+set 138; set 90+set 139; set 90+set 140; set 90+set 141; set 90+set 142; set 90+set 143; set 90+set 144; set 90+set 145; set 90+set 146; set 90+set 147; set 90+set 148; set 90+set 149; set 90+set 150; set 90+set 151; set 90+set 152; set 90+set 153; set 90+set 154; set 90+set 155; set 90+set 156; set 90+set 157; set 90+set 158; set 90+set 159; set 90+set 160; set 90+set 161; set 90+set 162; set 90+set 163; set 90+set 164; set 90+set 165; set 90+set 166; set 90+set 167; set 90+set 168; set 90+set 169;
Set 91+set 138; set 91+set 139; set 91+set 140; set 91+set 141; set 91+set 142; set 91+set 143; set 91+set 144; set 91+set 145; set 91+set 146; set 91+set 147; set 91+set 148; set 91+set 149; set 91+set 150; set 91+set 151; set 91+set 152; set 91+set 153; set 91+set 154; set 91+set 155; set 91+set 156; set 91+set 157; set 91+set 158; set 91+set 159; set 91+set 160; set 91+set 161; set 91+set 162; set 91+set 163; set 91+set 164; set 91+set 165; set 91+set 166; set 91+set 167; set 91+set 168; set 91+set 169;
Set 92+set 138; set 92+set 139; set 92+set 140; set 92+set 141; set 92+set 142; set 92+set 143; set 92+set 144; set 92+set 145; set 92+set 146; set 92+set 147; set 92+set 148; set 92+set 149; set 92+set 150; set 92+set 151; set 92+set 152; set 92+set 153; set 92+set 154; set 92+set 155; set 92+set 156; set 92+set 157; set 92+set 158; set 92+set 159; set 92+set 160; set 92+set 161; set 92+set 162; set 92+set 163; set 92+set 164; set 92+set 165; set 92+set 166; set 92+set 167; set 92+set 168; set 92+set 169;
Set 93+set 138; set 93+set 139; set 93+set 140; set 93+set 141; set 93+set 142; set 93+set 143; set 93+set 144; set 93+set 145; set 93+set 146; set 93+set 147; set 93+set 148; set 93+set 149; set 93+set 150; set 93+set 151; set 93+set 152; set 93+set 153; set 93+set 154; set 93+set 155; set 93+set 156; set 93+set 157; set 93+set 158; set 93+set 159; set 93+set 160; set 93+set 161; set 93+set 162; set 93+set 163; set 93+set 164; set 93+set 165; set 93+set 166; set 93+set 167; set 93+set 168; set 93+set 169;
Set 94+set 138; set 94+set 139; set 94+set 140; set 94+set 141; set 94+set 142; set 94+set 143; set 94+set 144; set 94+set 145; set 94+set 146; set 94+set 147; set 94+set 148; set 94+set 149; set 94+set 150; set 94+set 151; set 94+set 152; set 94+set 153; set 94+set 154; set 94+set 155; set 94+set 156; set 94+set 157; set 94+set 158; set 94+set 159; set 94+set 160; set 94+set 161; set 94+set 162; set 94+set 163; set 94+set 164; set 94+set 165; set 94+set 166; set 94+set 167; set 94+set 168; set 94+set 169;
Set 95+set 138; set 95+set 139; set 95+set 140; set 95+set 141; set 95+set 142; set 95+set 143; set 95+set 144; set 95+set 145; set 95+set 146; set 95+set 147; set 95+set 148; set 95+set 149; set 95+set 150; set 95+set 151; set 95+set 152; set 95+set 153; set 95+set 154; set 95+set 155; set 95+set 156; set 95+set 157; set 95+set 158; set 95+set 159; set 95+set 160; set 95+set 161; set 95+set 162; set 95+set 163; set 95+set 164; set 95+set 165; set 95+set 166; set 95+set 167; set 95+set 168; set 95+set 169;
Set 96+set 138; set 96+set 139; set 96+set 140; set 96+set 141; set 96+set 142; set 96+set 143; set 96+set 144; set 96+set 145; set 96+set 146; set 96+set 147; set 96+set 148; set 96+set 149; set 96+set 150; set 96+set 151; set 96+set 152; set 96+set 153; set 96+set 154; set 96+set 155; set 96+set 156; set 96+set 157; set 96+set 158; set 96+set 159; set 96+set 160; set 96+set 161; set 96+set 162; set 96+set 163; set 96+set 164; set 96+set 165; set 96+set 166; set 96+set 167; set 96+set 168; set 96+set 169;
Set 97+set 138; set 97+set 139; set 97+set 140; set 97+set 141; set 97+set 142; set 97+set 143; set 97+set 144; set 97+set 145; set 97+set 146; set 97+set 147; set 97+set 148; set 97+set 149; set 97+set 150; set 97+set 151; set 97+set 152; set 97+set 153; set 97+set 154; set 97+set 155; set 97+set 156; set 97+set 157; set 97+set 158; set 97+set 159; set 97+set 160; set 97+set 161; set 97+set 162; set 97+set 163; set 97+set 164; set 97+set 165; set 97+set 166; set 97+set 167; set 97+set 168; set 97+set 169;
Set 98+set 138; set 98+set 139; set 98+set 140; set 98+set 141; set 98+set 142; set 98+set 143; set 98+set 144; set 98+set 145; set 98+set 146; set 98+set 147; set 98+set 148; set 98+set 149; set 98+set 150; set 98+set 151; set 98+set 152; set 98+set 153; set 98+set 154; set 98+set 155; set 98+set 156; set 98+set 157; set 98+set 158; set 98+set 159; set 98+set 160; set 98+set 161; set 98+set 162; set 98+set 163; set 98+set 164; set 98+set 165; set 98+set 166; set 98+set 167; set 98+set 168; set 98+set 169;
Set 99+set 138; set 99+set 139; set 99+set 140; set 99+set 141; set 99+set 142; set 99+set 143; set 99+set 144; set 99+set 145; set 99+set 146; set 99+set 147; set 99+set 148; set 99+set 149; set 99+set 150; set 99+set 151; set 99+set 152; set 99+set 153; set 99+set 154; set 99+set 155; set 99+set 156; set 99+set 157; set 99+set 158; set 99+set 159; set 99+set 160; set 99+set 161; set 99+set 162; set 99+set 163; set 99+set 164; set 99+set 165; set 99+set 166; set 99+set 167; set 99+set 168; set 99+set 169;
Set 100+set 138; set 100+set 139; set 100+set 140; set 100+set 141; set 100+set 142; set 100+set 143; set 100+set 144; set 100+set 145; set 100+set 146; set 100+set 147; set 100+set 148; set 100+set 149; set 100+set 150; set 100+set 151; set 100+set 152; set 100+set 153; set 100+set 154; set 100+set 155; set 100+set 156; set 100+set 157; set 100+set 158; set 100+set 159; set 100+set 160; set 100+set 161; set 100+set 162; set 100+set 163; set 100+set 164; set 100+set 165; set 100+set 166; set 100+set 167; set 100+set 168; set 100+set 169;
Set 101+set 138; set 101+set 139; set 101+set 140; set 101+set 141; set 101+set 142; set 101+set 143; set 101+set 144; set 101+set 145; set 101+set 146; set 101+set 147; set 101+set 148; set 101+set 149; set 101+set 150; set 101+set 151; set 101+set 152; set 101+set 153; set 101+set 154; set 101+set 155; set 101+set 156; set 101+set 157; set 101+set 158; set 101+set 159; set 101+set 160; set 101+set 161; set 101+set 162; set 101+set 163; set 101+set 164; set 101+set 165; set 101+set 166; set 101+set 167; set 101+set 168; set 101+set 169;
Set 102+set 138; set 102+set 139; set 102+set 140; set 102+set 141; set 102+set 142; set 102+set 143; set 102+set 144; set 102+set 145; set 102+set 146; set 102+set 147; set 102+set 148; set 102+set 149; set 102+set 150; set 102+set 151; set 102+set 152; set 102+set 153; set 102+set 154; set 102+set 155; set 102+set 156; set 102+set 157; set 102+set 158; set 102+set 159; set 102+set 160; set 102+set 161; set 102+set 162; set 102+set 163; set 102+set 164; set 102+set 165; set 102+set 166; set 102+set 167; set 102+set 168; set 102+set 169;
Set 103+set 138; set 103+set 139; set 103+set 140; set 103+set 141; set 103+set 142; set 103+set 143; set 103+set 144; set 103+set 145; set 103+set 146; set 103+set 147; set 103+set 148; set 103+set 149; set 103+set 150; set 103+set 151; set 103+set 152; set 103+set 153; set 103+set 154; set 103+set 155; set 103+set 156; set 103+set 157; set 103+set 158; set 103+set 159; set 103+set 160; set 103+set 161; set 103+set 162; set 103+set 163; set 103+set 164; set 103+set 165; set 103+set 166; set 103+set 167; set 103+set 168; set 103+set 169;
Set 104+set 138; set 104+set 139; set 104+set 140; set 104+set 141; set 104+set 142; set 104+set 143; set 104+set 144; set 104+set 145; set 104+set 146; set 104+set 147; set 104+set 148; set 104+set 149; set 104+set 150; set 104+set 151; set 104+set 152; set 104+set 153; set 104+set 154; set 104+set 155; set 104+set 156; set 104+set 157; set 104+set 158; set 104+set 159; set 104+set 160; set 104+set 161; set 104+set 162; set 104+set 163; set 104+set 164; set 104+set 165; set 104+set 166; set 104+set 167; set 104+set 168; set 104+set 169;
Set 105+set 138; set 105+set 139; set 105+set 140; set 105+set 141; set 105+set 142; set 105+set 143; set 105+set 144; set 105+set 145; set 105+set 146; set 105+set 147; set 105+set 148; set 105+set 149; set 105+set 150; set 105+set 151; set 105+set 152; set 105+set 153; set 105+set 154; set 105+set 155; set 105+set 156; set 105+set 157; set 105+set 158; set 105+set 159; set 105+set 160; set 105+set 161; set 105+set 162; set 105+set 163; set 105+set 164; set 105+set 165; set 105+set 166; set 105+set 167; set 105+set 168; set 105+set 169;
Set 106+set 138; set 106+set 139; set 106+set 140; set 106+set 141; set 106+set 142; set 106+set 143; set 106+set 144; set 106+set 145; set 106+set 146; set 106+set 147; set 106+set 148; set 106+set 149; set 106+set 150; set 106+set 151; set 106+set 152; set 106+set 153; set 106+set 154; set 106+set 155; set 106+set 156; set 106+set 157; set 106+set 158; set 106+set 159; set 106+set 160; set 106+set 161; set 106+set 162; set 106+set 163; set 106+set 164; set 106+set 165; set 106+set 166; set 106+set 167; set 106+set 168; set 106+set 169;
Set 107+set 138; set 107+set 139; set 107+set 140; set 107+set 141; set 107+set 142; set 107+set 143; set 107+set 144; set 107+set 145; set 107+set 146; set 107+set 147; set 107+set 148; set 107+set 149; set 107+set 150; set 107+set 151; set 107+set 152; set 107+set 153; set 107+set 154; set 107+set 155; set 107+set 156; set 107+set 157; set 107+set 158; set 107+set 159; set 107+set 160; set 107+set 161; set 107+set 162; set 107+set 163; set 107+set 164; set 107+set 165; set 107+set 166; set 107+set 167; set 107+set 168; set 107+set 169;
Set 108+set 138; set 108+set 139; set 108+set 140; set 108+set 141; set 108+set 142; set 108+set 143; set 108+set 144; set 108+set 145; set 108+set 146; set 108+set 147; set 108+set 148; set 108+set 149; set 108+set 150; set 108+set 151; set 108+set 152; set 108+set 153; set 108+set 154; set 108+set 155; set 108+set 156; set 108+set 157; set 108+set 158; set 108+set 159; set 108+set 160; set 108+set 161; set 108+set 162; set 108+set 163; set 108+set 164; set 108+set 165; set 108+set 166; set 108+set 167; set 108+set 168; set 108+set 169;
Set 109+set 138; set 109+set 139; set 109+set 140; set 109+set 141; set 109+set 142; set 109+set 143; set 109+set 144; set 109+set 145; set 109+set 146; set 109+set 147; set 109+set 148; set 109+set 149; set 109+set 150; set 109+set 151; set 109+set 152; set 109+set 153; set 109+set 154; set 109+set 155; set 109+set 156; set 109+set 157; set 109+set 158; set 109+set 159; set 109+set 160; set 109+set 161; set 109+set 162; set 109+set 163; set 109+set 164; set 109+set 165; set 109+set 166; set 109+set 167; set 109+set 168; set 109+set 169;
Set 110+set 138; set 110+set 139; set 110+set 140; set 110+set 141; set 110+set 142; set 110+set 143; set 110+set 144; set 110+set 145; set 110+set 146; set 110+set 147; set 110+set 148; set 110+set 149; set 110+set 150; set 110+set 151; set 110+set 152; set 110+set 153; set 110+set 154; set 110+set 155; set 110+set 156; set 110+set 157; set 110+set 158; set 110+set 159; set 110+set 160; set 110+set 161; set 110+set 162; set 110+set 163; set 110+set 164; set 110+set 165; set 110+set 166; set 110+set 167; set 110+set 168; set 110+set 169;
Set 111+set 138; set 111+set 139; set 111+set 140; set 111+set 141; set 111+set 142; set 111+set 143; set 111+set 144; set 111+set 145; set 111+set 146; set 111+set 147; set 111+set 148; set 111+set 149; set 111+set 150; set 111+set 151; set 111+set 152; set 111+set 153; set 111+set 154; set 111+set 155; set 111+set 156; set 111+set 157; set 111+set 158; set 111+set 159; set 111+set 160; set 111+set 161; set 111+set 162; set 111+set 163; set 111+set 164; set 111+set 165; set 111+set 166; set 111+set 167; set 111+set 168; set 111+set 169;
Set 112+set 138; set 112+set 139; set 112+set 140; set 112+set 141; set 112+set 142; set 112+set 143; set 112+set 144; set 112+set 145; set 112+set 146; set 112+set 147; set 112+set 148; set 112+set 149; set 112+set 150; set 112+set 151; set 112+set 152; set 112+set 153; set 112+set 154; set 112+set 155; set 112+set 156; set 112+set 157; set 112+set 158; set 112+set 159; set 112+set 160; set 112+set 161; set 112+set 162; set 112+set 163; set 112+set 164; set 112+set 165; set 112+set 166; set 112+set 167; set 112+set 168; set 112+set 169;
Set 113+set 138; set 113+set 139; set 113+set 140; set 113+set 141; set 113+set 142; set 113+set 143; set 113+set 144; set 113+set 145; set 113+set 146; set 113+set 147; set 113+set 148; set 113+set 149; set 113+set 150; set 113+set 151; set 113+set 152; set 113+set 153; set 113+set 154; set 113+set 155; set 113+set 156; set 113+set 157; set 113+set 158; set 113+set 159; set 113+set 160; set 113+set 161; set 113+set 162; set 113+set 163; set 113+set 164; set 113+set 165; set 113+set 166; set 113+set 167; set 113+set 168; set 113+set 169;
Set 114+set 138; set 114+set 139; set 114+set 140; set 114+set 141; set 114+set 142; set 114+set 143; set 114+set 144; set 114+set 145; set 114+set 146; set 114+set 147; set 114+set 148; set 114+set 149; set 114+set 150; set 114+set 151; set 114+set 152; set 114+set 153; set 114+set 154; set 114+set 155; set 114+set 156; set 114+set 157; set 114+set 158; set 114+set 159; set 114+set 160; set 114+set 161; set 114+set 162; set 114+set 163; set 114+set 164; set 114+set 165; set 114+set 166; set 114+set 167; set 114+set 168; set 114+set 169;
Set 115+set 138; set 115+set 139; set 115+set 140; set 115+set 141; set 115+set 142; set 115+set 143; set 115+set 144; set 115+set 145; set 115+set 146; set 115+set 147; set 115+set 148; set 115+set 149; set 115+set 150; set 115+set 151; set 115+set 152; set 115+set 153; set 115+set 154; set 115+set 155; set 115+set 156; set 115+set 157; set 115+set 158; set 115+set 159; set 115+set 160; set 115+set 161; set 115+set 162; set 115+set 163; set 115+set 164; set 115+set 165; set 115+set 166; set 115+set 167; set 115+set 168; set 115+set 169;
Set 116+set 138; set 116+set 139; set 116+set 140; set 116+set 141; set 116+set 142; set 116+set 143; set 116+set 144; set 116+set 145; set 116+set 146; set 116+set 147; set 116+set 148; set 116+set 149; set 116+set 150; set 116+set 151; set 116+set 152; set 116+set 153; set 116+set 154; set 116+set 155; set 116+set 156; set 116+set 157; set 116+set 158; set 116+set 159; set 116+set 160; set 116+set 161; set 116+set 162; set 116+set 163; set 116+set 164; set 116+set 165; set 116+set 166; set 116+set 167; set 116+set 168; set 116+set 169;
Set 117+set 138; set 117+set 139; set 117+set 140; set 117+set 141; set 117+set 142; set 117+set 143; set 117+set 144; set 117+set 145; set 117+set 146; set 117+set 147; set 117+set 148; set 117+set 149; set 117+set 150; set 117+set 151; set 117+set 152; set 117+set 153; set 117+set 154; set 117+set 155; set 117+set 156; set 117+set 157; set 117+set 158; set 117+set 159; set 117+set 160; set 117+set 161; set 117+set 162; set 117+set 163; set 117+set 164; set 117+set 165; set 117+set 166; set 117+set 167; set 117+set 168; set 117+set 169;
Set 118+set 138; set 118+set 139; set 118+set 140; set 118+set 141; set 118+set 142; set 118+set 143; set 118+set 144; set 118+set 145; set 118+set 146; set 118+set 147; set 118+set 148; set 118+set 149; set 118+set 150; set 118+set 151; set 118+set 152; set 118+set 153; set 118+set 154; set 118+set 155; set 118+set 156; set 118+set 157; set 118+set 158; set 118+set 159; set 118+set 160; set 118+set 161; set 118+set 162; set 118+set 163; set 118+set 164; set 118+set 165; set 118+set 166; set 118+set 167; set 118+set 168; set 118+set 169;
Set 119+set 138; set 119+set 139; set 119+set 140; set 119+set 141; set 119+set 142; set 119+set 143; set 119+set 144; set 119+set 145; set 119+set 146; set 119+set 147; set 119+set 148; set 119+set 149; set 119+set 150; set 119+set 151; set 119+set 152; set 119+set 153; set 119+set 154; set 119+set 155; set 119+set 156; set 119+set 157; set 119+set 158; set 119+set 159; set 119+set 160; set 119+set 161; set 119+set 162; set 119+set 163; set 119+set 164; set 119+set 165; set 119+set 166; set 119+set 167; set 119+set 168; set 119+set 169;
Set 120+set 138; set 120+set 139; set 120+set 140; set 120+set 141; set 120+set 142; set 120+set 143; set 120+set 144; set 120+set 145; set 120+set 146; set 120+set 147; set 120+set 148; set 120+set 149; set 120+set 150; set 120+set 151; set 120+set 152; set 120+set 153; set 120+set 154; set 120+set 155; set 120+set 156; set 120+set 157; set 120+set 158; set 120+set 159; set 120+set 160; set 120+set 161; set 120+set 162; set 120+set 163; set 120+set 164; set 120+set 165; set 120+set 166; set 120+set 167; set 120+set 168; set 120+set 169;
Set 121+set 138; set 121+set 139; set 121+set 140; set 121+set 141; set 121+set 142; set 121+set 143; set 121+set 144; set 121+set 145; set 121+set 146; set 121+set 147; set 121+set 148; set 121+set 149; set 121+set 150; set 121+set 151; set 121+set 152; set 121+set 153; set 121+set 154; set 121+set 155; set 121+set 156; set 121+set 157; set 121+set 158; set 121+set 159; set 121+set 160; set 121+set 161; set 121+set 162; set 121+set 163; set 121+set 164; set 121+set 165; set 121+set 166; set 121+set 167; set 121+set 168; set 121+set 169;
Set 122+set 138; set 122+set 139; set 122+set 140; set 122+set 141; set 122+set 142; set 122+set 143; set 122+set 144; set 122+set 145; set 122+set 146; set 122+set 147; set 122+set 148; set 122+set 149; set 122+set 150; set 122+set 151; set 122+set 152; set 122+set 153; set 122+set 154; set 122+set 155; set 122+set 156; set 122+set 157; set 122+set 158; set 122+set 159; set 122+set 160; set 122+set 161; set 122+set 162; set 122+set 163; set 122+set 164; set 122+set 165; set 122+set 166; set 122+set 167; set 122+set 168; set 122+set 169;
Set 123+set 138; set 123+set 139; set 123+set 140; set 123+set 141; set 123+set 142; set 123+set 143; set 123+set 144; set 123+set 145; set 123+set 146; set 123+set 147; set 123+set 148; set 123+set 149; set 123+set 150; set 123+set 151; set 123+set 152; set 123+set 153; set 123+set 154; set 123+set 155; set 123+set 156; set 123+set 157; set 123+set 158; set 123+set 159; set 123+set 160; set 123+set 161; set 123+set 162; set 123+set 163; set 123+set 164; set 123+set 165; set 123+set 166; set 123+set 167; set 123+set 168; set 123+set 169;
Set 124+set 138; set 124+set 139; set 124+set 140; set 124+set 141; set 124+set 142; set 124+set 143; set 124+set 144; set 124+set 145; set 124+set 146; set 124+set 147; set 124+set 148; set 124+set 149; set 124+set 150; set 124+set 151; set 124+set 152; set 124+set 153; set 124+set 154; set 124+set 155; set 124+set 156; set 124+set 157; set 124+set 158; set 124+set 159; set 124+set 160; set 124+set 161; set 124+set 162; set 124+set 163; set 124+set 164; set 124+set 165; set 124+set 166; set 124+set 167; set 124+set 168; set 124+set 169;
Set 125+set 138; set 125+set 139; set 125+set 140; set 125+set 141; set 125+set 142; set 125+set 143; set 125+set 144; set 125+set 145; set 125+set 146; set 125+set 147; set 125+set 148; set 125+set 149; set 125+set 150; set 125+set 151; set 125+set 152; set 125+set 153; set 125+set 154; set 125+set 155; set 125+set 156; set 125+set 157; set 125+set 158; set 125+set 159; set 125+set 160; set 125+set 161; set 125+set 162; set 125+set 163; set 125+set 164; set 125+set 165; set 125+set 166; set 125+set 167; set 125+set 168; set 125+set 169;
Set 126+set 138; set 126+set 139; set 126+set 140; set 126+set 141; set 126+set 142; set 126+set 143; set 126+set 144; set 126+set 145; set 126+set 146; set 126+set 147; set 126+set 148; set 126+set 149; set 126+set 150; set 126+set 151; set 126+set 152; set 126+set 153; set 126+set 154; set 126+set 155; set 126+set 156; set 126+set 157; set 126+set 158; set 126+set 159; set 126+set 160; set 126+set 161; set 126+set 162; set 126+set 163; set 126+set 164; set 126+set 165; set 126+set 166; set 126+set 167; set 126+set 168; set 126+set 169;
Set 127+set 138; set 127+set 139; set 127+set 140; set 127+set 141; set 127+set 142; set 127+set 143; set 127+set 144; set 127+set 145; set 127+set 146; set 127+set 147; set 127+set 148; set 127+set 149; set 127+set 150; set 127+set 151; set 127+set 152; set 127+set 153; set 127+set 154; set 127+set 155; set 127+set 156; set 127+set 157; set 127+set 158; set 127+set 159; set 127+set 160; set 127+set 161; set 127+set 162; set 127+set 163; set 127+set 164; set 127+set 165; set 127+set 166; set 127+set 167; set 127+set 168; set 127+set 169;
Set 128+set 138; set 128+set 139; set 128+set 140; set 128+set 141; set 128+set 142; set 128+set 143; set 128+set 144; set 128+set 145; set 128+set 146; set 128+set 147; set 128+set 148; set 128+set 149; set 128+set 150; set 128+set 151; set 128+set 152; set 128+set 153; set 128+set 154; set 128+set 155; set 128+set 156; set 128+set 157; set 128+set 158; set 128+set 159; set 128+set 160; set 128+set 161; set 128+set 162; set 128+set 163; set 128+set 164; set 128+set 165; set 128+set 166; set 128+set 167; set 128+set 168; set 128+set 169;
Set 129+set 138; set 129+set 139; set 129+set 140; set 129+set 141; set 129+set 142; set 129+set 143; set 129+set 144; set 129+set 145; set 129+set 146; set 129+set 147; set 129+set 148; set 129+set 149; set 129+set 150; set 129+set 151; set 129+set 152; set 129+set 153; set 129+set 154; set 129+set 155; set 129+set 156; set 129+set 157; set 129+set 158; set 129+set 159; set 129+set 160; set 129+set 161; set 129+set 162; set 129+set 163; set 129+set 164; set 129+set 165; set 129+set 166; set 129+set 167; set 129+set 168; set 129+set 169;
Set 130+set 138; set 130+set 139; set 130+set 140; set 130+set 141; set 130+set 142; set 130+set 143; set 130+set 144; set 130+set 145; set 130+set 146; set 130+set 147; set 130+set 148; set 130+set 149; set 130+set 150; set 130+set 151; set 130+set 152; set 130+set 153; set 130+set 154; set 130+set 155; set 130+set 156; set 130+set 157; set 130+set 158; set 130+set 159; set 130+set 160; set 130+set 161; set 130+set 162; set 130+set 163; set 130+set 164; set 130+set 165; set 130+set 166; set 130+set 167; set 130+set 168; set 130+set 169;
Set 131+set 138; set 131+set 139; set 131+set 140; set 131+set 141; set 131+set 142; set 131+set 143; set 131+set 144; set 131+set 145; set 131+set 146; set 131+set 147; set 131+set 148; set 131+set 149; set 131+set 150; set 131+set 151; set 131+set 152; set 131+set 153; set 131+set 154; set 131+set 155; set 131+set 156; set 131+set 157; set 131+set 158; set 131+set 159; set 131+set 160; set 131+set 161; set 131+set 162; set 131+set 163; set 131+set 164; set 131+set 165; set 131+set 166; set 131+set 167; set 131+set 168; set 131+set 169;
Set 132+set 138; set 132+set 139; set 132+set 140; set 132+set 141; set 132+set 142; set 132+set 143; set 132+set 144; set 132+set 145; set 132+set 146; set 132+set 147; set 132+set 148; set 132+set 149; set 132+set 150; set 132+set 151; set 132+set 152; set 132+set 153; set 132+set 154; set 132+set 155; set 132+set 156; set 132+set 157; set 132+set 158; set 132+set 159; set 132+set 160; set 132+set 161; set 132+set 162; set 132+set 163; set 132+set 164; set 132+set 165; set 132+set 166; set 132+set 167; set 132+set 168; set 132+set 169;
Set 133+set 138; set 133+set 139; set 133+set 140; set 133+set 141; set 133+set 142; set 133+set 143; set 133+set 144; set 133+set 145; set 133+set 146; set 133+set 147; set 133+set 148; set 133+set 149; set 133+set 150; set 133+set 151; set 133+set 152; set 133+set 153; set 133+set 154; set 133+set 155; set 133+set 156; set 133+set 157; set 133+set 158; set 133+set 159; set 133+set 160; set 133+set 161; set 133+set 162; set 133+set 163; set 133+set 164; set 133+set 165; set 133+set 166; set 133+set 167; set 133+set 168; set 133+set 169;
Set 134+set 138; set 134+set 139; set 134+set 140; set 134+set 141; set 134+set 142; set 134+set 143; set 134+set 144; set 134+set 145; set 134+set 146; set 134+set 147; set 134+set 148; set 134+set 149; set 134+set 150; set 134+set 151; set 134+set 152; set 134+set 153; set 134+set 154; set 134+set 155; set 134+set 156; set 134+set 157; set 134+set 158; set 134+set 159; set 134+set 160; set 134+set 161; set 134+set 162; set 134+set 163; set 134+set 164; set 134+set 165; set 134+set 166; set 134+set 167; set 134+set 168; set 134+set 169;
Set 135+set 138; set 135+set 139; set 135+set 140; set 135+set 141; set 135+set 142; set 135+set 143; set 135+set 144; set 135+set 145; set 135+set 146; set 135+set 147; set 135+set 148; set 135+set 149; set 135+set 150; set 135+set 151; set 135+set 152; set 135+set 153; set 135+set 154; set 135+set 155; set 135+set 156; set 135+set 157; set 135+set 158; set 135+set 159; set 135+set 160; set 135+set 161; set 135+set 162; set 135+set 163; set 135+set 164; set 135+set 165; set 135+set 166; set 135+set 167; set 135+set 168; set 135+set 169;
Set 136+set 138; set 136+set 139; set 136+set 140; set 136+set 141; set 136+set 142; set 136+set 143; set 136+set 144; set 136+set 145; set 136+set 146; set 136+set 147; set 136+set 148; set 136+set 149; set 136+set 150; set 136+set 151; set 136+set 152; set 136+set 153; set 136+set 154; set 136+set 155; set 136+set 156; set 136+set 157; set 136+set 158; set 136+set 159; set 136+set 160; set 136+set 161; set 136+set 162; set 136+set 163; set 136+set 164; set 136+set 165; set 136+set 166; set 136+set 167; set 136+set 168; set 136+set 169;
Set 137+set 138; set 137+set 139; set 137+set 140; set 137+set 141; set 137+set 142; set 137+set 143; set 137+set 144; set 137+set 145; set 137+set 146; set 137+set 147; set 137+set 148; set 137+set 149; set 137+set 150; set 137+set 151; set 137+set 152; set 137+set 153; set 137+set 154; set 137+set 155; set 137+set 156; set 137+set 157; set 137+set 158; set 137+set 159; set 137+set 160; set 137+set 161; set 137+set 162; set 137+set 163; set 137+set 164; set 137+set 165; set 137+set 166; set 137+set 167; set 137+set 168; set 137+set 169.

In further embodiments, the invention provides (i) a vaccine comprising a plurality of peptides, or (ii) a method for treating an infection, disorder or disease caused by HPV-33, comprising administering to a human subject a plurality of peptides,

wherein said plurality of peptides comprises a set of peptides selected from the
182. the peptides set forth in SEQ ID NO: 136, 139, 140, 141, 142, 160 and 161,
183. the peptides set forth in SEQ ID NO: 137, 139, 140, 141, 142, 160 and 161,
184. the peptides set forth in SEQ ID NO: 138, 139, 140, 141, 142, 160 and 161,
185. the peptides set forth in SEQ ID NO: 136, 139, 140, 143, 144, 160 and 161,
186. the peptides set forth in SEQ ID NO: 137, 139, 140, 143, 144, 160 and 161,
187. the peptides set forth in SEQ ID NO: 138, 139, 140, 143, 144, 160 and 161,
188. the peptides set forth in SEQ ID NO: 136, 141, 145, 146, 147, 160 and 161,
189. the peptides set forth in SEQ ID NO: 137, 141, 145, 146, 147, 160 and 161,
190. the peptides set forth in SEQ ID NO: 138, 141, 145, 146, 147, 160 and 161,
191. the peptides set forth in SEQ ID NO: 136, 139, 140, 141, 142, 162 and 163,
192. the peptides set forth in SEQ ID NO: 137, 139, 140, 141, 142, 162 and 163,
193. the peptides set forth in SEQ ID NO: 138, 139, 140, 141, 142, 162 and 163,
194. the peptides set forth in SEQ ID NO: 136, 139, 140, 143, 144, 162 and 163,
195. the peptides set forth in SEQ ID NO: 137, 139, 140, 143, 144, 162 and 163,
196. the peptides set forth in SEQ ID NO: 138, 139, 140, 143, 144, 162 and 163,
197. the peptides set forth in SEQ ID NO: 136, 141, 145, 146, 147, 162 and 163,
198. the peptides set forth in SEQ ID NO: 137, 141, 145, 146, 147, 162 and 163,
199. the peptides set forth in SEQ ID NO: 138, 141, 145, 146, 147, 162 and 163,
200. the peptides set forth in SEQ ID NO: 136, 139, 140, 141, 142, 160 and 162,
201. the peptides set forth in SEQ ID NO: 137, 139, 140, 141, 142, 160 and 162,
202. the peptides set forth in SEQ ID NO: 138, 139, 140, 141, 142, 160 and 162,
203. the peptides set forth in SEQ ID NO: 136, 139, 140, 143, 144, 160 and 162,
204. the peptides set forth in SEQ ID NO: 137, 139, 140, 143, 144, 160 and 162,
205. the peptides set forth in SEQ ID NO: 138, 139, 140, 143, 144, 160 and 162,
206. the peptides set forth in SEQ ID NO: 136, 141, 145, 146, 147, 160 and 162,
207. the peptides set forth in SEQ ID NO: 137, 141, 145, 146, 147, 160 and 162,
208. the peptides set forth in SEQ ID NO: 138, 141, 145, 146, 147, 160 and 162.

In further embodiments, the invention provides (i) a vaccine comprising a plurality of peptides, or (ii) a method for treating an infection, disorder or disease caused by HPV-33, comprising administering to a human subject a plurality of peptides,

wherein said plurality of peptides comprises a set of peptides selected from the following sets:
209. the peptides set forth in SEQ ID NO:166, 167, 177 and 178,
210. the peptides set forth in SEQ ID NO:167, 168, 169, 177 and 178,
211. the peptides set forth in SEQ ID NO:167, 170, 177 and 178,
212. the peptides set forth in SEQ ID NO:166, 167, 179 and 180,
213. the peptides set forth in SEQ ID NO:167, 168, 169, 179 and 180,
214. the peptides set forth in SEQ ID NO:167, 170, 179 and 180,
215. the peptides set forth in SEQ ID NO: 166, 167, 181 and 182,
216. the peptides set forth in SEQ ID NO:167, 168, 169, 181 and 182,
217. the peptides set forth in SEQ ID NO:167, 170, 181 and 182,
218. the peptides set forth in SEQ ID NO:166, 167, 185, 177 and 178,
219. the peptides set forth in SEQ ID NO:167, 168, 169, 185, 177 and 178,
220. the peptides set forth in SEQ ID NO:167, 170, 185, 177 and 178,
221. the peptides set forth in SEQ ID NO:166, 167, 185, 179 and 180,
222. the peptides set forth in SEQ ID NO:167, 168, 169, 185, 179 and 180,
223. the peptides set forth in SEQ ID NO:167, 170, 185, 179 and 180,
224. the peptides set forth in SEQ ID NO:166, 167, 185, 181 and 182,
225. the peptides set forth in SEQ ID NO:167, 168, 169, 185, 181 and 182,
226. the peptides set forth in SEQ ID NO:167, 170, 185, 181 and 182.

In further embodiments, the invention provides (i) a vaccine comprising a plurality of peptides, or (ii) a method for treating an infection, disorder or disease caused by HPV-33, comprising administering to a human subject a plurality of peptides,

wherein said plurality of peptides comprises a set of peptides selected from the following combinations of sets:
Set 182+set 209; set 182+set 210; set 182+set 211; set 182+set 212; set 182+set 213; set 182+set 214; set 182+set 215; set 182+set 216; set 182+set 217; set 182+set 218; set 182+set 219; set 182+set 220; set 182+set 221; set 182+set 222; set 182+set 223; set 182+set 224; set 182+set 225; set 182+set 226;
Set 183+set 209; set 183+set 210; set 183+set 211; set 183+set 212; set 183+set 213; set 183+set 214; set 183+set 215; set 183+set 216; set 183+set 217; set 183+set 218; set 183+set 219; set 183+set 220; set 183+set 221; set 183+set 222; set 183+set 223; set 183+set 224; set 183+set 225; set 183+set 226,
Set 184+set 209; set 184+set 210; set 184+set 211; set 184+set 212; set 184+set 213; set 184+set 214; set 184+set 215; set 184+set 216; set 184+set 217; set 184+set 218; set 184+set 219; set 184+set 220; set 184+set 221; set 184+set 222; set 184+set 223; set 184+set 224; set 184+set 225; set 184+set 226,
Set 185+set 209; set 185+set 210; set 185+set 211; set 185+set 212; set 185+set 213; set 185+set 214; set 185+set 215; set 185+set 216; set 185+set 217; set 185+set 218; set 185+set 219; set 185+set 220; set 185+set 221; set 185+set 222; set 185+set 223; set 185+set 224; set 185+set 225; set 185+set 226;
Set 186+set 209; set 186+set 210; set 186+set 211; set 186+set 212; set 186+set 213; set 186+set 214; set 186+set 215; set 186+set 216; set 186+set 217; set 186+set 218; set 186+set 219; set 186+set 220; set 186+set 221; set 186+set 222; set 186+set 223; set 186+set 224; set 186+set 225; set 186+set
Set 187+set 209; set 187+set 210; set 187+set 211; set 187+set 212; set 187+set 213; set 187+set 214; set 187+set 215; set 187+set 216; set 187+set 217; set 187+set 218; set 187+set 219; set 187+set 220; set 187+set 221; set 187+set 222; set 187+set 223; set 187+set 224; set 187+set 225; set 187+set 226,
Set 188+set 209; set 188+set 210; set 188+set 211; set 188+set 212; set 188+set 213; set 188+set 214; set 188+set 215; set 188+set 216; set 188+set 217; set 188+set 218; set 188+set 219; set 188+set 220; set 188+set 221; set 188+set 222; set 188+set 223; set 188+set 224; set 188+set 225; set 188+set 226;
Set 189+set 209; set 189+set 210; set 189+set 211; set 189+set 212; set 189+set 213; set 189+set 214; set 189+set 215; set 189+set 216; set 189+set 217; set 189+set 218; set 189+set 219; set 189+set 220; set 189+set 221; set 189+set 222; set 189+set 223; set 189+set 224; set 189+set 225; set 189+set 226;
Set 190+set 209; set 190+set 210; set 190+set 211; set 190+set 212; set 190+set 213; set 190+set 214; set 190+set 215; set 190+set 216; set 190+set 217; set 190+set 218; set 190+set 219; set 190+set 220; set 190+set 221; set 190+set 222; set 190+set 223; set 190+set 224; set 190+set 225; set 190+set 226,
Set 191+set 209; set 191+set 210; set 191+set 211; set 191+set 212; set 191+set 213; set 191+set 214; set 191+set 215; set 191+set 216; set 191+set 217; set 191+set 218; set 191+set 219; set 191+set 220; set 191+set 221; set 191+set 222; set 191+set 223; set 191+set 224; set 191+set 225; set 191+set 226;
Set 192+set 209; set 192+set 210; set 192+set 211; set 192+set 212; set 192+set 213; set 192+set 214; set 192+set 215; set 192+set 216; set 192+set 217; set 192+set 218; set 192+set 219; set 192+set 220; set 192+set 221; set 192+set 222; set 192+set 223; set 192+set 224; set 192+set 225; set 192+set 226;
Set 193+set 209; set 193+set 210; set 193+set 211; set 193+set 212; set 193+set 213; set 193+set 214; set 193+set 215; set 193+set 216; set 193+set 217; set 193+set 218; set 193+set 219; set 193+set 220; set 193+set 221; set 193+set 222; set 193+set 223; set 193+set 224; set 193+set 225; set 193+set 226;
Set 194+set 209; set 194+set 210; set 194+set 211; set 194+set 212; set 194+set 213; set 194+set 214; set 194+set 215; set 194+set 216; set 194+set 217; set 194+set 218; set 194+set 219; set 194+set 220; set 194+set 221; set 194+set 222; set 194+set 223; set 194+set 224; set 194+set 225; set 194+set 226;
Set 195+set 209; set 195+set 210; set 195+set 211; set 195+set 212; set 195+set 213; set 195+set 214; set 195+set 215; set 195+set 216; set 195+set 217; set 195+set 218; set 195+set 219; set 195+set 220; set 195+set 221; set 195+set 222; set 195+set 223; set 195+set 224; set 195+set 225; set 195+set 226,
Set 196+set 209; set 196+set 210; set 196+set 211; set 196+set 212; set 196+set 213; set 196+set 214; set 196+set 215; set 196+set 216; set 196+set 217; set 196+set 218; set 196+set 219; set 196+set 220; set 196+set 221; set 196+set 222; set 196+set 223; set 196+set 224; set 196+set 225; set 196+set 226;
Set 197+set 209; set 197+set 210; set 197+set 211; set 197+set 212; set 197+set 213; set 197+set 214; set 197+set 215; set 197+set 216; set 197+set 217; set 197+set 218; set 197+set 219; set 197+set 220; set 197+set 221; set 197+set 222; set 197+set 223; set 197+set 224; set 197+set 225; set 197+set 226;
Set 198+set 209; set 198+set 210; set 198+set 211; set 198+set 212; set 198+set 213; set 198+set 214; set 198+set 215; set 198+set 216; set 198+set 217; set 198+set 218; set 198+set 219; set 198+set 220; set 198+set 221; set 198+set 222; set 198+set 223; set 198+set 224; set 198+set 225; set 198+set 226;
Set 199+set 209; set 199+set 210; set 199+set 211; set 199+set 212; set 199+set 213; set 199+set 214; set 199+set 215; set 199+set 216; set 199+set 217; set 199+set 218; set 199+set 219; set 199+set 220; set 199+set 221; set 199+set 222; set 199+set 223; set 199+set 224; set 199+set 225; set 199+set 226;
Set 200+set 209; set 200+set 210; set 200+set 211; set 200+set 212; set 200+set 213; set 200+set 214; set 200+set 215; set 200+set 216; set 200+set 217; set 200+set 218; set 200+set 219; set 200+set 220; set 200+set 221; set 200+set 222; set 200+set 223; set 200+set 224; set 200+set 225; set 200+set 226;
Set 201+set 209; set 201+set 210; set 201+set 211; set 201+set 212; set 201+set 213; set 201+set 214; set 201+set 215; set 201+set 216; set 201+set 217; set 201+set 218; set 201+set 219; set 201+set 220; set 201+set 221; set 201+set 222; set 201+set 223; set 201+set 224; set 201+set 225; set 201+set 226;
Set 202+set 209; set 202+set 210; set 202+set 211; set 202+set 212; set 202+set 213; set 202+set 214; set 202+set 215; set 202+set 216; set 202+set 217; set 202+set 218; set 202+set 219; set 202+set 220; set 202+set 221; set 202+set 222; set 202+set 223; set 202+set 224; set 202+set 225; set 202+set 226
Set 203+set 209; set 203+set 210; set 203+set 211; set 203+set 212; set 203+set 213; set 203+set 214; set 203+set 215; set 203+set 216; set 203+set 217; set 203+set 218; set 203+set 219; set 203+set 220; set 203+set 221; set 203+set 222; set 203+set 223; set 203+set 224; set 203+set 225; set 203+set 226;
Set 204+set 209; set 204+set 210; set 204+set 211; set 204+set 212; set 204+set 213; set 204+set 214; set 204+set 215; set 204+set 216; set 204+set 217; set 204+set 218; set 204+set 219; set 204+set 220; set 204+set 221; set 204+set 222; set 204+set 223; set 204+set 224; set 204+set 225; set 204+set 226;
Set 205+set 209; set 205+set 210; set 205+set 211; set 205+set 212; set 205+set 213; set 205+set 214; set 205+set 215; set 205+set 216; set 205+set 217; set 205+set 218; set 205+set 219; set 205+set 220; set 205+set 221; set 205+set 222; set 205+set 223; set 205+set 224; set 205+set 225; set 205+set 226;
Set 206+set 209; set 206+set 210; set 206+set 211; set 206+set 212; set 206+set 213; set 206+set 214; set 206+set 215; set 206+set 216; set 206+set 217; set 206+set 218; set 206+set 219; set 206+set 220; set 206+set 221; set 206+set 222; set 206+set 223; set 206+set 224; set 206+set 225; set 206+set 226;
Set 207+set 209; set 207+set 210; set 207+set 211; set 207+set 212; set 207+set 213; set 207+set 214; set 207+set 215; set 207+set 216; set 207+set 217; set 207+set 218; set 207+set 219; set 207+set 220; set 207+set 221; set 207+set 222; set 207+set 223; set 207+set 224; set 207+set 225; set 207+set 226,
Set 208+set 209; set 208+set 210; set 208+set 211; set 208+set 212; set 208+set 213; set 208+set 214; set 208+set 215; set 208+set 216; set 208+set 217; set 208+set 218; set 208+set 219; set 208+set 220; set 208+set 221; set 208+set 222; set 208+set 223; set 208+set 224; set 208+set 225; set 208+set 226.

In further embodiments, the invention provides (i) a vaccine comprising a plurality of peptides, or (ii) a method for treating an infection, disorder or disease caused by HPV-52, comprising administering to a human subject a plurality of peptides,

wherein said plurality of peptides comprises a set of peptides selected from the following sets:
227. the peptides set forth in SEQ ID NO: 188, 191, 192, 193, 194, 195, 196, 205 and 206,
228. the peptides set forth in SEQ ID NO: 189, 191, 192, 193, 194, 195, 196, 205 and 206,
229. the peptides set forth in SEQ ID NO: 190, 191, 192, 193, 194, 195, 196, 205 and 206,
230. the peptides set forth in SEQ ID NO: 188, 193, 194, 197, 198, 199, 200, 205 and 206,
231. the peptides set forth in SEQ ID NO: 189, 193, 194, 197, 198, 199, 200, 205 and 206,
232. the peptides set forth in SEQ ID NO: 190, 193, 194, 197, 198, 199, 200, 205 and 206,
233. the peptides set forth in SEQ ID NO: 188, 195, 196, 201, 202, 203, 204, 205 and 206,
234. the peptides set forth in SEQ ID NO: 189, 195, 196, 201, 202, 203, 204, 205 and 206,
235. the peptides set forth in SEQ ID NO: 190, 195, 196, 201, 202, 203, 204, 205 and 206,
236. the peptides set forth in SEQ ID NO: 188, 191, 192, 193, 194, 195, 196, 206 and 207,
237. the peptides set forth in SEQ ID NO: 189, 191, 192, 193, 194, 195, 196, 206 and 207,
238. the peptides set forth in SEQ ID NO: 190, 191, 192, 193, 194, 195, 196, 206 and 207,
239. the peptides set forth in SEQ ID NO: 188, 193, 194, 197, 198, 199, 200, 206 and 207,
240. the peptides set forth in SEQ ID NO: 189, 193, 194, 197, 198, 199, 200, 206 and 207,
241. the peptides set forth in SEQ ID NO: 190, 193, 194, 197, 198, 199, 200, 206 and 207,
242. the peptides set forth in SEQ ID NO: 188, 195, 196, 201, 202, 203, 204, 206 and 207,
243. the peptides set forth in SEQ ID NO: 189, 195, 196, 201, 202, 203, 204, 206 and 207,
244. the peptides set forth in SEQ ID NO: 190, 195, 196, 201, 202, 203, 204, 206 and 207,
245. the peptides set forth in SEQ ID NO: 188, 191, 192, 193, 194, 195, 196, 206 and 208,
246. the peptides set forth in SEQ ID NO: 189, 191, 192, 193, 194, 195, 196, 206 and 208,
247. the peptides set forth in SEQ ID NO: 190, 191, 192, 193, 194, 195, 196, 206 and 208,
248. the peptides set forth in SEQ ID NO: 188, 193, 194, 197, 198, 199, 200, 206 and 208,
249. the peptides set forth in SEQ ID NO: 189, 193, 194, 197, 198, 199, 200, 206 and 208,
250. the peptides set forth in SEQ ID NO: 190, 193, 194, 197, 198, 199, 200, 206 and 208,
251. the peptides set forth in SEQ ID NO: 188, 195, 196, 201, 202, 203, 204, 206 and 208,
252. the peptides set forth in SEQ ID NO: 189, 195, 196, 201, 202, 203, 204, 206 and 208,
253. the peptides set forth in SEQ ID NO: 190, 195, 196, 201, 202, 203, 204, 206 and 208.

In further embodiments, the invention provides (i) a vaccine comprising a plurality of peptides, or (ii) a method for treating an infection, disorder or disease caused by HPV-52, comprising administering to a human subject a plurality of peptides,

wherein said plurality of peptides comprises a set of peptides selected from the following sets:
254. the peptides set forth in SEQ ID NO: 209, 210, 211, 216 and 217,
255. the peptides set forth in SEQ ID NO: 210, 212, 213, 216 and 217,
256. the peptides set forth in SEQ ID NO: 211, 214, 215, 216 and 217,
257. the peptides set forth in SEQ ID NO: 209, 210, 211, 218 and 219,
258. the peptides set forth in SEQ ID NO: 210, 212, 213, 218 and 219,
259. the peptides set forth in SEQ ID NO: 211, 214, 215, 218 and 219,
260. the peptides set forth in SEQ ID NO: 209, 210, 211, 219 and 220,
261. the peptides set forth in SEQ ID NO: 210, 212, 213, 219 and 220,
262. the peptides set forth in SEQ ID NO: 211, 214, 215, 219 and 220.

In further embodiments, the invention provides (i) a vaccine comprising a plurality of peptides, or (ii) a method for treating an infection, disorder or disease caused by HPV-52, comprising administering to a human subject a plurality of peptides,

wherein said plurality of peptides comprises a set of peptides selected from the following combinations of sets:

### Set 227+set 254; set 227+set 255; set 227+set 256; set 227+set 257; set 227+set 258; set 227+set 259; set 227+set 260; set 227+set 261; set 227+set 262,

### Set 228+set 254; set 228+set 255; set 228+set 256; set 228+set 257; set 228+set 258; set 228+set 259; set 228+set 260; set 228+set 261; set 228+set 262;

### Set 229+set 254; set 229+set 255; set 229+set 256; set 229+set 257; set 229+set 258; set 229+set 259; set 229+set 260; set 229+set 261; set 229+set 262;

### Set 230+set 254; set 230+set 255; set 230+set 256; set 230+set 257; set 230+set 258; set 230+set 259; set 230+set 260; set 230+set 261; set 230+set 262,

### Set 231+set 254; set 231+set 255; set 231+set 256; set 231+set 257; set 231+set 258; set 231+set 259; set 231+set 260; set 231+set 261; set 231+set 262;

### Set 232+set 254; set 232+set 255; set 232+set 256; set 232+set 257; set 232+set 258; set 232+set 259; set 232+set 260; set 232+set 261; set 232+set 262,

### Set 233+set 254; set 233+set 255; set 233+set 256; set 233+set 257; set 233+set 258; set 233+set 259; set 233+set 260; set 233+set 261; set 233+set 262,

### Set 234+set 254; set 234+set 255; set 234+set 256; set 234+set 257; set 234+set 258; set 234+set 259; set 234+set 260; set 234+set 261; set 234+set 262;

### Set 235+set 254; set 235+set 255; set 235+set 256; set 235+set 257; set 235+set 258; set 235+set 259; set 235+set 260; set 235+set 261; set 235+set 262;

### Set 236+set 254; set 236+set 255; set 236+set 256; set 236+set 257; set 236+set 258; set 236+set 259; set 236+set 260; set 236+set 261; set 236+set 262;

### Set 237+set 254; set 237+set 255; set 237+set 256; set 237+set 257; set 237+set 258; set 237+set 259; set 237+set 260; set 237+set 261; set 237+set 262;

### Set 238+set 254; set 238+set 255; set 238+set 256; set 238+set 257; set 238+set 258; set 238+set 259; set 238+set 260; set 238+set 261; set 238+set 262;

### Set 239+set 254; set 239+set 255; set 239+set 256; set 239+set 257; set 239+set 258; set 239+set 259; set 239+set 260; set 239+set 261; set 239+set 262;

### Set 240+set 254; set 240+set 255; set 240+set 256; set 240+set 257; set 240+set 258; set 240+set 259; set 240+set 260; set 240+set 261; set 240+set 262;

### Set 241+set 254; set 241+set 255; set 241+set 256; set 241+set 257; set 241+set 258; set 241+set 259; set 241+set 260; set 241+set 261; set 241+set 262;

### Set 242+set 254; set 242+set 255; set 242+set 256; set 242+set 257; set 242+set 258; set 242+set 259; set 242+set 260; set 242+set 261; set 242+set 262;

### Set 243+set 254; set 243+set 255; set 243+set 256; set 243+set 257; set 243+set 258; set 243+set 259; set 243+set 260; set 243+set 261; set 243+set 262;

### Set 244+set 254; set 244+set 255; set 244+set 256; set 244+set 257; set 244+set 258; set 244+set 259; set 244+set 260; set 244+set 261; set 244+set 262,

### Set 245+set 254; set 245+set 255; set 245+set 256; set 245+set 257; set 245+set 258; set 245+set 259; set 245+set 260; set 245+set 261; set 245+set 262;

### Set 246+set 254; set 246+set 255; set 246+set 256; set 246+set 257; set 246+set 258; set 246+set 259; set 246+set 260; set 246+set 261; set 246+set 262;

### Set 247+set 254; set 247+set 255; set 247+set 256; set 247+set 257; set 247+set 258; set 247+set 259; set 247+set 260; set 247+set 261; set 247+set 262

### Set 248+set 254; set 248+set 255; set 248+set 256; set 248+set 257; set 248+set 258; set 248+set 259; set 248+set 260; set 248+set 261; set 248+set 262;

### Set 249+set 254; set 249+set 255; set 249+set 256; set 249+set 257; set 249+set 258; set 249+set 259; set 249+set 260; set 249+set 261; set 249+set 262;

### Set 250+set 254; set 250+set 255; set 250+set 256; set 250+set 257; set 250+set 258; set 250+set 259; set 250+set 260; set 250+set 261; set 250+set 262;

### Set 251+set 254; set 251+set 255; set 251+set 256; set 251+set 257; set 251+set 258; set 251+set 259; set 251+set 260; set 251+set 261; set 251+set 262;

### Set 252+set 254; set 252+set 255; set 252+set 256; set 252+set 257; set 252+set 258; set 252+set 259; set 252+set 260; set 252+set 261; set 252+set 262;

### Set 253+set 254; set 253+set 255; set 253+set 256; set 253+set 257; set 253+set 258; set 253+set 259; set 253+set 260; set 253+set 261; set 253+set 262.

In further embodiments, the invention provides (i) a vaccine comprising a plurality of peptides, or (ii) a method for treating an infection, disorder or disease caused by HPV-31, comprising administering to a human subject a plurality of peptides,

wherein said plurality of peptides comprises a set of peptides selected from the following sets:
263. the peptides set forth in SEQ ID NO: 223, 224, 228, 229, 230, 234 and 235,
264. the peptides set forth in SEQ ID NO: 225, 226, 228, 229, 230, 234 and 235,
265. the peptides set forth in SEQ ID NO: 224, 226, 228, 229, 230, 234 and 235,
266. the peptides set forth in SEQ ID NO: 223, 224, 231, 232, 234 and 235,
267. the peptides set forth in SEQ ID NO: 225, 226, 231, 232, 234 and 235,
268. the peptides set forth in SEQ ID NO: 224, 226, 231, 232, 234 and 235,
269. the peptides set forth in SEQ ID NO: 223, 224, 229, 230, 233, 234 and 235,
270. the peptides set forth in SEQ ID NO: 225, 226, 229, 230, 233, 234 and 235,
271. the peptides set forth in SEQ ID NO: 224, 226, 229, 230, 233, 234 and 235,
272. the peptides set forth in SEQ ID NO: 223, 224, 228, 229, 230, 234 and 236,
273. the peptides set forth in SEQ ID NO: 225, 226, 228, 229, 230, 234 and 236,
274. the peptides set forth in SEQ ID NO: 224, 226, 228, 229, 230, 234 and 236,
275. the peptides set forth in SEQ ID NO: 223, 224, 231, 232, 234 and 236,
276. the peptides set forth in SEQ ID NO: 225, 226, 231, 232, 234 and 236,
277. the peptides set forth in SEQ ID NO: 224, 226, 231, 232, 234 and 236,
278. the peptides set forth in SEQ ID NO: 223, 224, 229, 230, 233, 234 and 236,
279. the peptides set forth in SEQ ID NO: 225, 226, 229, 230, 233, 234 and 236,
280. the peptides set forth in SEQ ID NO: 224, 226, 229, 230, 233, 234 and 236,
281. the peptides set forth in SEQ ID NO: 223, 224, 228, 229, 230, 235 and 237,
282. the peptides set forth in SEQ ID NO: 225, 226, 228, 229, 230, 235 and 237,
283. the peptides set forth in SEQ ID NO: 224, 226, 228, 229, 230, 235 and 237,
284. the peptides set forth in SEQ ID NO: 223, 224, 231, 232, 235 and 237,
285. the peptides set forth in SEQ ID NO: 225, 226, 231, 232, 235 and 237,
286. the peptides set forth in SEQ ID NO: 224, 226, 231, 232, 235 and 237,
287. the peptides set forth in SEQ ID NO: 223, 224, 229, 230, 233, 235 and 237,
288. the peptides set forth in SEQ ID NO: 225, 226, 229, 230, 233, 235 and 237,
289. the peptides set forth in SEQ ID NO: 224, 226, 229, 230, 233, 235 and 237.

In further embodiments, the invention provides (i) a vaccine comprising a plurality of peptides, or (ii) a method for treating an infection, disorder or disease caused by HPV-31, comprising administering to a human subject a plurality of peptides,

wherein said plurality of peptides comprises a set of peptides selected from the following sets:
290. the peptides set forth in SEQ ID NO: 238, 239, 240, 244 and 245,
291. the peptides set forth in SEQ ID NO: 241, 242, 244 and 245,
292. the peptides set forth in SEQ ID NO: 239, 240, 243, 244 and 245,
293. the peptides set forth in SEQ ID NO: 238, 239, 240 and 246,
294. the peptides set forth in SEQ ID NO: 241, 242 and 246,
295. the peptides set forth in SEQ ID NO: 239, 240, 243 and 246,
296. the peptides set forth in SEQ ID NO: 238, 239, 240 and 247,
297. the peptides set forth in SEQ ID NO: 241, 242 and 247,
298. the peptides set forth in SEQ ID NO: 239, 240, 243 and 247,
299. the peptides set forth in SEQ ID NO: 238, 239, 240, 248 and 249,
300. the peptides set forth in SEQ ID NO: 241, 242, 248 and 249,
301. the peptides set forth in SEQ ID NO: 239, 240, 243, 248 and 249,
302. the peptides set forth in SEQ ID NO: 238, 239, 240 and 250,
303. the peptides set forth in SEQ ID NO: 241, 242 and 250,
304. the peptides set forth in SEQ ID NO: 239, 240, 243 and 250.

In further embodiments, the invention provides (i) a vaccine comprising a plurality of peptides, or (ii) a method for treating an infection, disorder or disease caused by HPV-31, comprising administering to a human subject a plurality of peptides,

wherein said plurality of peptides comprises a set of peptides selected from the following combinations of sets:
Set 263+set 290; set 263+set 291; set 263+set 292; set 263+set 293; set 263+set 294; set 263+set 295; set 263+set 296; set 263+set 297; set 263+set 298; set 263+set 299; set 263+set 300; set 263+set 301; set 263+set 302; set 263+set 303; set 263+set 304;
Set 264+set 290; set 264+set 291; set 264+set 292; set 264+set 293; set 264+set 294; set 264+set 295; set 264+set 296; set 264+set 297; set 264+set 298; set 264+set 299; set 264+set 300; set 264+set 301; set 264+set 302; set 264+set 303; set 264+set 304;
Set 265+set 290; set 265+set 291; set 265+set 292; set 265+set 293; set 265+set 294; set 265+set 295; set 265+set 296; set 265+set 297; set 265+set 298; set 265+set 299; set 265+set 300; set 265+set 301; set 265+set 302; set 265+set 303; set 265+set 304;
Set 266+set 290; set 266+set 291; set 266+set 292; set 266+set 293; set 266+set 294; set 266+set 295; set 266+set 296; set 266+set 297; set 266+set 298; set 266+set 299; set 266+set 300; set 266+set 301; set 266+set 302; set 266+set 303; set 266+set 304;
Set 267+set 290; set 267+set 291; set 267+set 292; set 267+set 293; set 267+set 294; set 267+set 295; set 267+set 296; set 267+set 297; set 267+set 298; set 267+set 299; set 267+set 300; set 267+set 301; set 267+set 302; set 267+set 303; set 267+set 304;
Set 268+set 290; set 268+set 291; set 268+set 292; set 268+set 293; set 268+set 294; set 268+set 295; set 268+set 296; set 268+set 297; set 268+set 298; set 268+set 299; set 268+set 300; set 268+set 301; set 268+set 302; set 268+set 303; set 268+set 304;
Set 269+set 290; set 269+set 291; set 269+set 292; set 269+set 293; set 269+set 294; set 269+set 295; set 269+set 296; set 269+set 297; set 269+set 298; set 269+set 299; set 269+set 300; set 269+set 301; set 269+set 302; set 269+set 303; set 269+set 304;
Set 270+set 290; set 270+set 291; set 270+set 292; set 270+set 293; set 270+set 294; set 270+set 295; set 270+set 296; set 270+set 297; set 270+set 298; set 270+set 299; set 270+set 300; set 270+set 301; set 270+set 302; set 270+set 303; set 270+set 304;
Set 271+set 290; set 271+set 291; set 271+set 292; set 271+set 293; set 271+set 294; set 271+set 295; set 271+set 296; set 271+set 297; set 271+set 298; set 271+set 299; set 271+set 300; set 271+set 301; set 271+set 302; set 271+set 303; set 271+set 304;
Set 272+set 290; set 272+set 291; set 272+set 292; set 272+set 293; set 272+set 294; set 272+set 295; set 272+set 296; set 272+set 297; set 272+set 298; set 272+set 299; set 272+set 300; set 272+set 301; set 272+set 302; set 272+set 303; set 272+set 304;
Set 273+set 290; set 273+set 291; set 273+set 292; set 273+set 293; set 273+set 294; set 273+set 295; set 273+set 296; set 273+set 297; set 273+set 298; set 273+set 299; set 273+set 300; set 273+set 301; set 273+set 302; set 273+set 303; set 273+set 304;
Set 274+set 290; set 274+set 291; set 274+set 292; set 274+set 293; set 274+set 294; set 274+set 295; set 274+set 296; set 274+set 297; set 274+set 298; set 274+set 299; set 274+set 300; set 274+set 301; set 274+set 302; set 274+set 303; set 274+set 304;
Set 275+set 290; set 275+set 291; set 275+set 292; set 275+set 293; set 275+set 294; set 275+set 295; set 275+set 296; set 275+set 297; set 275+set 298; set 275+set 299; set 275+set 300; set 275+set 301; set 275+set 302; set 275+set 303; set 275+set 304;
Set 276+set 290; set 276+set 291; set 276+set 292; set 276+set 293; set 276+set 294; set 276+set 295; set 276+set 296; set 276+set 297; set 276+set 298; set 276+set 299; set 276+set 300; set 276+set 301; set 276+set 302; set 276+set 303; set 276+set 304;
Set 277+set 290; set 277+set 291; set 277+set 292; set 277+set 293; set 277+set 294; set 277+set 295; set 277+set 296; set 277+set 297; set 277+set 298; set 277+set 299; set 277+set 300; set 277+set 301; set 277+set 302; set 277+set 303; set 277+set 304;
Set 278+set 290; set 278+set 291; set 278+set 292; set 278+set 293; set 278+set 294; set 278+set 295; set 278+set 296; set 278+set 297; set 278+set 298; set 278+set 299; set 278+set 300; set 278+set 301; set 278+set 302; set 278+set 303; set 278+set 304;
Set 279+set 290; set 279+set 291; set 279+set 292; set 279+set 293; set 279+set 294; set 279+set 295; set 279+set 296; set 279+set 297; set 279+set 298; set 279+set 299; set 279+set 300; set 279+set 301; set 279+set 302; set 279+set 303; set 279+set 304;
Set 280+set 290; set 280+set 291; set 280+set 292; set 280+set 293; set 280+set 294; set 280+set 295; set 280+set 296; set 280+set 297; set 280+set 298; set 280+set 299; set 280+set 300; set 280+set 301; set 280+set 302; set 280+set 303; set 280+set 304;
Set 281+set 290; set 281+set 291; set 281+set 292; set 281+set 293; set 281+set 294; set 281+set 295; set 281+set 296; set 281+set 297; set 281+set 298; set 281+set 299; set 281+set 300; set 281+set 301; set 281+set 302; set 281+set 303; set 281+set 304;
Set 282+set 290; set 282+set 291; set 282+set 292; set 282+set 293; set 282+set 294; set 282+set 295; set 282+set 296; set 282+set 297; set 282+set 298; set 282+set 299; set 282+set 300; set 282+set 301; set 282+set 302; set 282+set 303; set 282+set 304;
Set 283+set 290; set 283+set 291; set 283+set 292; set 283+set 293; set 283+set 294; set 283+set 295; set 283+set 296; set 283+set 297; set 283+set 298; set 283+set 299; set 283+set 300; set 283+set 301; set 283+set 302; set 283+set 303; set 283+set 304;
Set 284+set 290; set 284+set 291; set 284+set 292; set 284+set 293; set 284+set 294; set 284+set 295; set 284+set 296; set 284+set 297; set 284+set 298; set 284+set 299; set 284+set 300; set 284+set 301; set 284+set 302; set 284+set 303; set 284+set 304;
Set 285+set 290; set 285+set 291; set 285+set 292; set 285+set 293; set 285+set 294; set 285+set 295; set 285+set 296; set 285+set 297; set 285+set 298; set 285+set 299; set 285+set 300; set 285+set 301; set 285+set 302; set 285+set 303; set 285+set 304;
Set 286+set 290; set 286+set 291; set 286+set 292; set 286+set 293; set 286+set 294; set 286+set 295; set 286+set 296; set 286+set 297; set 286+set 298; set 286+set 299; set 286+set 300; set 286+set 301; set 286+set 302; set 286+set 303; set 286+set 304;
Set 287+set 290; set 287+set 291; set 287+set 292; set 287+set 293; set 287+set 294; set 287+set 295; set 287+set 296; set 287+set 297; set 287+set 298; set 287+set 299; set 287+set 300; set 287+set 301; set 287+set 302; set 287+set 303; set 287+set 304;
Set 288+set 290; set 288+set 291; set 288+set 292; set 288+set 293; set 288+set 294; set 288+set 295; set 288+set 296; set 288+set 297; set 288+set 298; set 288+set 299; set 288+set 300; set 288+set 301; set 288+set 302; set 288+set 303; set 288+set 304;
Set 289+set 290; set 289+set 291; set 289+set 292; set 289+set 293; set 289+set 294; set 289+set 295; set 289+set 296; set 289+set 297; set 289+set 298; set 289+set 299; set 289+set 300; set 289+set 301; set 289+set 302; set 289+set 303; set 289+set 304.

In a further aspect, the invention relates to an immunogenic composition comprising one or more peptides selected from the group of peptides set forth in SEQ ID NO:1, 2, 3, 130, 48, 50, 52, 54, 56, 58, 60 and 63.

In an even further aspect, the invention relates to a method for treating an infection, disorder or disease caused by HPV-18, comprising administration to a subject of one or more peptides selected from the group of peptides set forth in: SEQ ID NO:1, 2, 3, 130, 48, 50, 52, 54, 56, 58, 60 and 63.

In one embodiment, the method comprises administration of:

i) the peptide set forth in SEQ ID NO:1 and the peptide set forth in SEQ ID NO:130, or

ii) the peptide set forth in SEQ ID NO:2 and the peptide set forth in SEQ ID NO:130, or

iii) the peptide set forth in SEQ ID NO:3 and the peptide set forth in SEQ ID NO:130.

In another embodiment, the method further comprises administration of a plurality of HPV-18 E6 and/or E7 peptides as set forth herein.

### Immunogenic Compositions

Immunogenic compositions used in the invention are preferably for, and therefore formulated to be suitable for, administration to a human subject. Preferably, the administration is parenteral, e.g. intravenous, subcutaneous, intramuscular, intradermal intracutaneous and/or intratumoral administration, i.e. by injection.

The immunogenic compositions are preferably chemically stable, i.e. the peptides in the composition do not chemically degrade or decompose. Thus, preferably, the amount of un-degraded, un-decomposed and/or unreacted peptides within the solution and/or composition is at least 90%, 91%, 92%, 93%, 94%, 95%, 96%, 97%, 98%, 99% or even 100% by weight as compared to its original, after storage of the solution or liquid composition for at least about 0.5, 1, 1.5, 2 or at least 3 hours at room temperature. Chemical stability can be assessed using any suitable technique known in the art, for instance using UPLC/MS as exemplified herein. When using UPLC/MS, a solution/composition is defined as chemically stable if the total % area of peaks that do not represent the desired peptide product in the UV chromatogram after storage of at least about 0.5, 1, 1.5, 2 or at least 3 hours at room temperature is at most 20%, 15%, 10%, 9%, 8%, 7%, 6%, 5%, 4%, 3%, 2%, 1%, or 0% as compared to its original.

The immunogenic compositions are preferably also physically stable, i.e. the peptides in the composition do not precipitate or re-disperse. Physical stability can be assessed using any suitable technique known in the art, for instance by visual inspection or by particle distribution using a Malvern Mastersizer as exemplified herein, wherein average particle size is expressed in D(0.5). When using Malvern Mastersizer for assessing physical stability as exemplified herein, a solution/composition is defined as physically stable if the average D (0.5) after storage of at least about 0.5, 1, 1.5, 2 or at least 3 hours at room temperature is increased at most 50%, 40%, 30%, 20%, 10% or 5% as compared to its original (i.e. the freshly prepared solution directly after preparation). Preferably, a solution/composition is defined as physically stable if the average D(0.5) after storage of 3 hours at room temperature is increased at most 50%, 40%, 30%, 20%, 10% or 5%, preferably at most 20%, as compared to its original.

In one embodiment, the immunogenic composition comprises or consists of a mixture of dry or lyophilized peptides that are to be administered together.

### Adjuvants

In one embodiment, a composition used in the invention further comprises an adjuvant or the treatment in step d) further includes administration of an adjuvant. The term “adjuvant” is used herein to refer to substances that have immune-potentiating effects and are co-administered, or added to, or co-formulated with an antigenic agent in order to enhance, induce, elicit, and/or modulate the immunological response against the antigenic agent when administered to a subject. In one embodiment, the adjuvant is physically linked, such as covalently linked, to the peptide(s) to be reconstituted.

In one embodiment, the adjuvant is an oil-based adjuvant. Oil-based adjuvants can be used to form emulsions (e.g. water-in-oil or oil-in-water emulsions) and are appreciated in the art to enhance and direct the immune response. Preferably the oil-based adjuvant is a mineral oil-based adjuvant. Non-limiting examples of oil-based adjuvants are bio-based oil adjuvants (based on vegetable oil/fish oil, etc.), squalene-based adjuvant (e.g. MF59), Syntex Adjuvant Formulation (SAF; Lidgate, Deborah M, Preparation of the Syntex Adjuvant Formulation (SAF, SAF-m, and SAF-1), In: Vaccine Adjuvants, Volume 42 of the series Methods in Molecular Medicine™ p 229-237, ISSN1543-1894), Freund's Complete Adjuvant (FCA), Freund's Incomplete Adjuvant (FIA), adjuvants based on peanut oil (e.g. Adjuvant 65), Lipovant (Byars, N. E., Allison, A. C., 1990. Immunologic adjuvants: general properties, advantages, and limitations. In: Zola, H. (Ed.), Laboratory Methods in Immunology. p 39-51), ASO4 (A. Tagliabue, R. Rappuoli Vaccine adjuvants: the dream becomes real Hum. Vaccine, 4 (5), 2008, p 347-349), Montanide adjuvants, which are based on purified squalene and squalene emulsified with highly purified mannide mono-oleate (e.g. Montanide ISA 25 VG, 28 VG, 35 VG, 50 V, 50 V2, 51 VG, 61 VG, 70 VG, 70 M VG, 71 VG, 720 VG, 760 VG, 763 A VG, 775 VG, 780 VG, 201 VG, 206 VG, 207 VG). More preferably, the oil-based adjuvant is Montanide ISA 51VG (Seppic), which is a mixture of Drakeol VR and mannide monooleate.

Other suitable adjuvants are adjuvants that are known to act via the Toll-like receptors and/or via a RIG-I (Retinoic acid-Inducible Gene-1) protein and/or via an endothelin receptor. Immune modifying compounds that are capable of activation of the innate immune system can be activated particularly well via Toll like receptors (TLRs), including TLRs 1-10. Compounds capable of activating TLR receptors and modifications and derivatives thereof are well documented in the art. TLR1 may be activated by bacterial lipoproteins and acetylated forms thereof, TLR2 may in addition be activated by Gram positive bacterial glycolipids, LPS, LPA, LTA, fimbriae, outer membrane proteins, heat shock proteins from bacteria or from the host, and Mycobacterial lipoarabinomannans. TLR3 may be activated by dsRNA, in particular of viral origin, or by the chemical compound poly(I:C). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLR5 may be activated with bacterial flagellae or flagellin. TLR6 may be activated by mycobacterial lipoproteins and group B Streptococcus heat labile soluble factor (GBS-F) or Staphylococcus modulins. TLR7 may be activated by imidazoquinolines, such as imiquimod, resiquimod and derivatives imiquimod or resiquimod (e.g. 3M-052). TLR9 may be activated by unmethylated CpG DNA or chromatin—IgG complexes. Particularly preferred adjuvants comprise, but are not limited to, synthetically produced compounds comprising dsRNA, poly(I:C), poly I:CLC, unmethylated CpG DNA which trigger TLR3 and TLR9 receptors, IC31, a TLR 9 agonist, IMSAVAC, a TLR4 agonist, Montanide ISA-51, Montanide ISA 720 (an adjuvant produced by Seppic, France). RIG-I protein is known to be activated by ds-RNA just like TLR3 (Kato et al, (2005) Immunity, 1: 19-28).

A further particularly preferred TLR ligand is a pam3cys and/or derivative thereof, preferably a pam3cys lipopeptide or variant or derivative thereof, preferably such as described in WO2013051936A1, more preferably U-Pam12 or U-Pam14 or AMPLIVANT®. Pam3cys and/or derivatives thereof may optionally be covalently linked to the peptide antigen(s).

Further preferred adjuvants are Cyclic dinucleotides (CDNs), Muramyl dipeptide (MDP) and poly-ICLC. In a preferred embodiment, the adjuvants of the invention are non-naturally occurring adjuvants such as the pam3cys lipopeptide derivative as described in WO2013051936A1, Poly-ICLC, imidazoquinoline such as imiquimod, resiquimod or derivatives thereof, CpG oligodeoxynucleotides (CpG-ODNs) having a non-naturally occurring sequence, and peptide-based adjuvants, such as muramyl dipeptide (MDP) or tetanus toxoid peptide, comprising non-naturally occurring amino acids.

Further preferred are adjuvants selected from the group consisting of: 1018 ISS, aluminum salts, Amplivax, AS 15, BCG, CP-870,893, CpG7909, CyaA, dSLIM, GM-CSF, IC30, IC31, ImuFact EV1P321, IS Patch, ISS, ISCOMATRIX, Juvlmmune, LipoVac, MF59, monophosphoryl lipid A, Montanide IMS 1312, OK-432, OM-174, OM-197-MP-EC, ONTAK, PepTel™, vector system, PLGA microparticles, SRL172, Virosomes and other Virus-like particles, Pam3Cys-GDPKHPKSF, YF-17D, VEGF trap, R848, beta-glucan, Aquila's QS21 stimulon, vadimezan, AsA404 (DMXAA), STING (stimulator of IFN genes) agonist (e.g. c-di-GMP VacciGrade™), PCI, NKT (natural killer T cell) agonist (e.g. alpha-galactosylceramide or alpha-GalCer, RNAdjuvant® (Curevac), retinoic acid inducible protein I ligands (e.g. 3pRNA or 5′-triphosphate RNA).

### Dosages

Preferably, the vaccine or immunogenic composition comprises or consists of an amount of peptides that constitutes a pharmaceutical dose. A pharmaceutical dose is defined herein as the amount of active ingredients (i.e. the total amounts of peptides in a peptide-based vaccine) that is applied to a subject at a given time point. A pharmaceutical dose may be applied to a subject in a single volume, i.e. a single shot, or may be applied in 2, 3, 4, 5 or more separate volumes that are applied preferably at different locations of the body, for instance in the right and the left limb. Reasons for applying a single pharmaceutical dose in separate volumes may be multiples, such as avoid negative side effects, avoiding antigenic competition and/or composition analytics considerations.

A pharmaceutical dose may be an effective amount or part of an effective amount. An “effective amount” is to be understood herein as an amount or dose of active ingredients required to prevent and/or reduce the symptoms of a disease (e.g., chronic infection, pre-cancerous condition and/or cancer) relative to an untreated patient. The effective amount of active compound(s) used to practice the present invention for preventive and/or therapeutic treatment of a disease or condition varies depending upon the manner of administration, the age, body weight, and general health of the subject. Ultimately, the attending physician or veterinarian will decide the appropriate amount and dosage regimen. Such amount is referred to as an “effective” amount. This effective amount may also be the amount that is able to induce an effective cellular T cell response in the subject to be treated, or more preferably an effective systemic cellular T cell response.

Preferably, pharmaceutical dose, or total amount of peptides applied to a subject at a given time point, either in a single or in multiple injections at a certain time point, comprises an amount of peptides in the range from 0.1 μg to 20 mg, such as about 0.1 μg, 0.5 μg, 1 μg, 5 μg, 10 μg, 15 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 650 μg, 700 μg, 750 μg, 800 μg, 850 μg, 900 μg, 1 mg, 1.1 mg, 1.2 mg, 1.3 mg, 1.4 mg, 1.5 mg, 1.6 mg, 1.7 mg, 1.8 mg, 1.9 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg or about 20 mg or any value in between. Preferred ranges of pharmaceutical doses are from 0.1 μg to 20 mg, 1 μg to 10 mg, 10 μg to 5 mg, 0.5 mg to 2 mg, 0.5 mg to 10 mg or 1 mg to 5 mg or 2 to 4 mg.

In one embodiment, the vaccine or immunogenic composition used in the invention is administered in a dose of between 1 microgram and 300 micrograms, e.g. between 50 micrograms and 150 micrograms, such as approximately 100 micrograms of each peptide.

A single injection volume (i.e. volume applied on one location at a certain time point), comprising a total pharmaceutical dose, may between 100 μL and 2 mL, or between 100 μL and 1 mL. The single injection volume may be 100 μL, 200 μL, 300 μL, 400 μL, 500 μL, 600 μL, 700 μL, 800 μL, 900 μL, 1 mL, 1.1 mL, 1.2 mL, 1.3 mL, 1.4 mL, 1.5 mL, 1.6 mL, 1.7 mL, 1.8 mL, 1.9 mL, 2 mL, 3 mL or any value in between.

### Conditions to be Treated

Examples of diseases or disorders to be treated via the method of the invention or using the vaccines of the invention include, without limitation, warts, genital warts, Recurrent Respiratory Papillomatosis (RRP), cervical intraepithelial neoplasia (CIN), Vulvar intraepithelial neoplasia (VIN), vaginal intraepithelial neoplasia (VaIN), anal intraepithelial neoplasia (AIN), and penal intraepithelial neoplasia (PIN), adenocarcinoma in situ (AIS) as well as cancer of the cervix, vulva, vagina, anus, penis, aerodigestive tract, and head & neck, including cancer of the tongue, nasal cavity, oral cavity, salivary gland, paranasal sinuses, larynx, pharynx (nasopharynx, oropharynx, hypopharynx).

### Medical Treatment and Uses

As described above, the method of the invention involves treating a human subject with immunogenic composition(s) and/or treating said human subject with a population of antigen-loaded activated antigen presenting cells (APCs) or a population of expanded antigen-specific T cells, wherein said population of cells has been generated ex vivo using said immunogenic composition(s).

Thus, in one embodiment, the use or medical treatment comprises a step of administration of a vaccine or one or more immunogenic composition(s) comprising a plurality of peptides, wherein each of said peptides comprises or consists of a contiguous fragment of 18-95 amino acids in length of the sequence of the E6 or the E7 protein of the HPV type identified in the sample, wherein the contiguous E6 and E7 fragments comprised within the plurality of peptides together in total cover at least 70% of the combined E6 and E7 sequences.

As mentioned, the plurality of peptides may all be comprised within one immunogenic composition, or the plurality of peptides may be divided over two or more compositions. If the peptides are divided over two or more compositions, these compositions may be mixed prior to administration and thus be co-administered, or they may be administered separately. Typically, all compositions, and thus all peptides of the plurality of peptides will be administered to the subject in a time interval of 24 hours, preferably within 4, 2 or 1 hour.

If two or more compositions are administered, the administration may be at the same site, e.g. in the same limb, or at two or more different sites. In the course of the treatment, the administration of the composition(s) may be carried out once or alternatively may be repeated (boosted) subsequently, such as, but not limited to, twice or three times.

Preferably, the composition is administered in an effective amount as defined herein above. Preferably, administration is intravenous or subcutaneous, or intramuscular administration, although other administration routes can be envisaged, such as mucosal administration or intradermal and/or intracutaneous administration or intratumoral administration, e.g., by injection.

Preferably, the administration of the vaccine or composition(s) induce(s) a T cell response against at least one epitope comprised in a peptide. Preferably, the administration is for the prevention, partial clearance and/or treatment or full clearance of an HPV-related infection, disorder or disease in a subject, e.g. a persistent infection, cancerous (neoplasia) or precancerous disorder, preferably as detectable by:
activation or an induction of the immune system and/or an increase in antigen-specific activated CD4+ and/or CD8+ T-cells in peripheral blood or in tissues as established by Elispot assay or by tetramer staining of CD4+ or CD8+ T cells or an increase of the cytokines produced by these T-cells as established by intracellular cytokine staining of CD4+ and CD8+ T cells in flow cytometry after at least one week of treatment; and/or
inhibition of proliferation of antigen-related infection or a detectable decrease of antigen-expressing cells or a decrease in cell viability of antigen-expressing cells; and/or
induction or increased induction of cell death of antigen-expressing cells; and/or
inhibition or prevention of the increase of antigen-expressing cells; and/or
decrease of viral load (premalignant stages of the disease); and/or
decrease of the reference lesion size; and/or
decrease of the reference tumor size; and/or
increase of survival rates (e.g. PFS, OS).

The method of the invention may be part of a combination therapy, which may be provided as a separate treatment or added to the immunogenic composition of the invention. The method of the invention may be combined with drugs that inhibit or block the inhibitory immune checkpoint molecules (e.g. A2AR (Adenosine A2A receptor), B7-H3/CD276, B7-H4/VTCN1, BTLA/CD272, CTLA-4/CD152, IDO (indoleamine 2,3-dioxygenase), KIR (Killer-cell Immunoglobulin-like Receptor), LAG3 (Lymphocyte Activation Gene-3), NOX2 (nicotinamide adenine dinucleotide phosphate NADPH oxidase isoform 2), PD-1 (Programmed Death 1), PD-L1, TIM-3 (T-cell Immunoglobulin domain and Mucin domain 3), VISTA (V-domain Ig suppressor of T cell activation), SIGLEC7/CD328 and SIGLEC9/CD329, NKG2A and/or drugs that stimulate stimulatory checkpoint molecules (e.g. selected Tumor Necrosis Factor (TNF) receptor superfamily members (e.g. CD27, CD40, CD122, 4-1BB/CD137, OX-40/CD134 and GITR (Glucocorticoid-Induced TNFR family Related)), CD28 and ICOS/CD278), immunosuppressive cytokines (e.g. IL-10, TGF-β and IL-6) and/or γC cytokines (e.g. IL-7, IL-15, and IL-21 or IL-2), thalidomide and/or derivatives thereof, further immunomodulators (e.g. compounds that are known to deplete immunosuppressive Tregs and/or MDSCs), standard of care treatment, e.g. chemotherapy, radiotherapy, chemoradation therapy, surgery, laser ablation, LLETZ (large loop excision of the transformation zone), exconization, cryotherapy, IFN-α conditioning, antiviral therapy, antibacterial therapy, UV therapy, anti-inflammatory therapy, anti-angiogenesis therapy etc. In case of the treatment or prevention of a pre-cancerous disorder or a cancer, the peptide-based vaccine may be combined with the relevant standard of care therapy, e.g. radiotherapy and/or chemotherapy such as treatment with carboplatin, paclitaxel, CarboTaxol (a combination of carboplatin, paclitaxel) and/or cisplatin, possibly combined with anti-angiogenesis therapy, such as anti-VEGF therapy. For example, the method of the invention may be part of a chemotherapy regimen wherein chemotherapy is applied once every three weeks. Preferably, a first pharmaceutical dosage unit of a pharmaceutical composition of the invention is administered two weeks after the second or third cycle of chemotherapy. The method of the invention may be part of an immunomodulatory regimen wherein checkpoint blocking therapy is applied once every 2 or 3 or 4 weeks. Preferably, a first pharmaceutical dosage unit of a pharmaceutical composition of the invention is administered 2-4 weeks before the start and/or simultaneous to the immunomodulatory therapy for a certain period of time.

The method of the invention may also be part of a regimen wherein chemotherapy is combined with immunomodulatory therapy.

In a preferred embodiment, the method of the invention is combined with a checkpoint blocker, such as one of the checkpoint blockers mentioned above, and optionally chemotherapy. In another preferred embodiment, the method of the invention is combined with anti-angiogenic therapy, such as anti-VEGF therapy, and optionally chemotherapy.

In another preferred embodiment, the method of the invention is combined with a tyrosine kinase inhibitor, and optionally chemotherapy.

In other embodiments, treatment with an immunogenic composition of the invention is combined with treatment with an inhibitor of an endothelin receptor such as BQ-788 (Buckanovich R J et al., (2008) Nature Medicine 14: 28; Ishikawa K, (1994) PNAS 91: 4892), or interferon alpha (IFNα), more preferably pegylated Interferon alpha. Furthermore, the use of antigen presenting cell (co)stimulatory molecules, as set out in WO99/61065 and in WO03/084999, in combination with the compositions of the invention is preferred. In particular the use of 4-1BB and/or CD40 ligands, agonistic antibodies, OX40 ligands, CD27 ligands or functional fragments and derivatives thereof, as well as synthetic compounds with similar agonistic activity are preferably administered separately or combined with the compositions of the invention to subjects to be treated in order to further stimulate the mounting of an optimal immune response in the subject.

As described above, in another embodiment, the method of the invention involves treating the human subject with a population of antigen-loaded activated antigen presenting cells (APCs) or expanded antigen-specific T cells, wherein said cells have been generated ex vivo (i.e. outside the body) using the immunogenic composition(s) described herein. This can e.g. be done by culturing HPV-typed cancer patients' PBMCs to generate autologous activated APCs (e.g. DCs), loaded with the immunogenic compositions (i.e. antigen-loaded APCs), and subsequently stimulate and expand HPV-type E6 or E7 antigen-specific T cells obtained from PBMCs or Tumor Infiltrating Lymphocytes. Alternatively, antigen-specific T cells may be expanded by incubation with activated APCs cultured from PBMCs of HLA-matched healthy donors, loaded with the immunogenic compositions. Suitable techniques have been described in the art, e.g. in McCormack et al. (2018) Cytotherapy 20:385; Stevanovic et al. (2015) J Clin Oncol 33:1543; and Stevanovic et al. (2018) Clin Cancer Res, doi: 10.1158/1078-0432.

In a further embodiment, treatment with a population of activated antigen presenting cells (APCs) or expanded antigen-specific T cells is combined with direct immunization of the human subject with the immunogenic compositions described herein. Such a combined protocol may involve sequential and/or simultaneous administrations.

### Preparation of Immunogenic Compositions

Vaccine or immunogenic compositions for use in the invention may be prepared by any suitable method. In some embodiment, the immunogenic composition(s) are prepared from dried, preferably lyophilized, peptides.

For example, the composition may be prepared by a method comprising the following steps:
a) providing a vial comprising dried, preferably lyophilized, peptides;
b) thawing the peptides, preferably for about 5-30 min;
c) adding a reconstitution composition to the vial comprising the peptides, preferably without swirling the vial;
d) allowing to admix, preferably for about 0.5-5 minutes; and
e) swirling until a clear solution is obtained, preferably for about 1-3 minutes.
   Preferably, steps b) to e) are performed at room temperature.

Preferably, said vial comprises peptides in an amount for injection as a single volume in a method for prevention and/or treatment, preferably a method of treatment and/or prevention as defined herein, i.e. a single pharmaceutical dosage unit, or part thereof in case of multiple injections at difference locations of the subject's body at substantially the same time point.

In one embodiment, the reconstitution composition of step c) comprises or consists of DMSO and/or water-for-injection. In another embodiment, the reconstitution composition of step c) of the method for reconstituting peptides comprises or consists of about 60-80% v/v aqueous solution comprising an organic acid, about 5-10% v/v propylene glycol (CAS no. 57-55-6), about 10-20% v/v lower alcohol and about 5-10% v/v non-ionic hydrophilic surfactant. In one embodiment, the organic acid is citric acid and the citric acid is present in the aqueous solution in a concentration of about 0.05-0.1M. In one embodiment, the lower alcohol is ethanol. In one embodiment, the non-ionic hydrophilic surfactant:

a. is a mono-, di or triglyceride, preferably an ethoxylated triglyceride, and/or
b. has a hydrophilic-lipophilic balance (HLB) value between 9 and 14. In a further embodiment, the non-ionic hydrophilic surfactant is ethoxylated castor oil, preferably polyoxyethyleneglyceroltriricinoleate 35 (CAS no. 61791-12-6).

In one embodiment, the composition comprises or consists of about 75% v/v aqueous solution comprising about 0.1M citric acid, about 6.25% v/v propylene glycol (CAS no. 57-55-6), about 12.5% v/v ethanol and about 6.25% v/v polyoxyethyleneglyceroltriricinoleate 35 (CAS no. 61791-12-6).

Preferably, the amount of reconstitution composition in step c) is in a range of from about 0.5 and 2 mL, preferably 1 mL. Preferably, the amount of reconstituted peptides in step (a) is the total amount of reconstituted peptides as obtained after step e), i.e. within the clear solution obtained after step e).

In one embodiment, the reconstituted composition comprises or consists of about 1-2 mg/mL peptides, 0.038M citric acid, about 3.13% v/v propylene glycol (CAS no. 57-55-6), about 6.25% v/v ethanol, about 3.13% v/v polyoxyethyleneglyceroltriricinoleate 35 (CAS no. 61791-12-6) and about 50% of an oil-based adjuvant, preferably Montanide ISA 51 VG (Seppic), in water.

Dried peptides may be peptides free of further constituents but may also comprise buffer components such as trifluoroacetic add (TFA), salts such as sodium, potassium or phosphate salts (e.g. NaCl, KCl and NaPO₄). The amount of further constituents is preferably less than 30%, more preferably less than 25%, of the total weight of the dry peptides to be reconstituted. Dried peptides to be reconstituted may be in a physical dried state as can be obtained by processes such as, but not limited to, rotor evaporation, lyophilization (freeze drying) and spray drying.

All patent and literature references cited in the present specification are hereby incorporated by reference in their entirety.

### EXAMPLE

### Introduction

Sets of SLPs (synthetic long peptides) with good manufacturability were identified. When combined, these sets cover E6 and E7 for more than 70% of a specific HPV-type. The amino acid lengths of E6 and E7 are HPV-type specific, but in general E6 comprises more than 137 amino acids and E7 comprises more than 85 amino acids. Multiple SLPs are required to form a set of SLPs that cover most epitopes. The total number of possible SLPs with a length of 18 to 35 amino acids for E6 and E7 together is more than 3000 for HPV-18, HPV-31, HPV-33, HPV-45 or HPV-52. Moreover, to cover more than 70% of the E6 and E7 proteins in a vaccine, combinations of these possible 3000 SLPs would be needed, which would result in an even larger number of possible options. However, the manufacturability of SLPs is highly sequence-dependent and if SLPs would be chosen randomly, many of them would turn out not be manufacturable due to low yield. A single SLP within the E6 or E7 sequence with good manufacturability can be identified by trial and error. However, to cover at least 70% of the combined E6 and E7 sequences, a vaccine composition of SLPs is required consisting of several SLPs with an acceptable manufacturability for all individual SLPs. In addition, the manufacturability of SLPs for certain regions of the E6 and E7 sequences are very poor due to sequence-specific properties such as high hydrophobicity. To identify a SLP with an acceptable manufacturability in these regions by trial and error is not easy. Therefore, an educated selection in advance of synthesis is required to identify an ideal set of SLPs which is suitable for manufacturing without the requirement of numerous attempts and/or optimizations.

The design of SLPs began by predicting the manufacturability of the SLPs of a certain length in silico by a machine learning algorithm. This prediction model had been trained by chemical properties (e.g. SLP length, hydrophobicity) of 361 unique data points. To train the prediction model, training SLPs were synthesized, the crude quality, i.e. quality of unpurified material, was determined by UPLC-MS and subsequently used as response variable. The UV-chromatogram is integrated in the specific range of interest, resulting in a total area under the curve (100%). The crude quality percentage is the total area under the curve minus the area corresponding to other products (i.e. the % of the area that corresponds to the desired product). Crude quality is an indication for yield of purified product, thus a measure for manufacturability of a given SLP. Next, ideal SLPs were selected by removing SLPs which are prone to degradation (i.e. loss of the original features of the purified peptide batch) based on their amino acid sequence. Peptides that contain one or more cysteines are susceptible to oxidation and may form disulfide bonds. Although the degree of oxidation during synthesis may be suppressed by introduction of thiol protecting groups, oxidation after the thiol deprotection step is difficult to control. Such features presumably affect the stability of synthesized peptide batches, rendering them less attractive for drug development. Therefore, SLPs with more than two cysteines or a cysteine at the N- or C-terminal or at both N- and C-terminals were not prioritized. Oxidation of the thioether moiety of methionines during manufacturing or storage may result into a sulfoxide or sulfone moiety. To minimalize this reaction, cleavage and deprotection was performed under an inert atmosphere and SLPs with more than three methionines were excluded of the ideal SLP sets. Lastly, peptides with an N-terminal glutamine were not prioritized for the ideal SLP sets as these SLPs are prone to pyroglutamate formation in the acidic aqueous media used during purification.

After ranking the residual SLPs according to the predicted manufacturability results various sets of SLPs with (near) optimal manufacturability were identified. Generally, the selected SLP sets consist of SLPs with a minimum overlap of 14 amino acids between adjacent SLPs in order to cover all possible CD4+ and CD8+epitopes and to enable proper proteasomal cleavage of C-terminal epitopes. Furthermore, a feasible therapeutically vaccine from a manufacturing, formulation and patient administration point of view will comprise a limited number of components without omitting components that are expected to be essential for the biological purpose. Therefore, SLP sets with a minimum number of SLPs were chosen.

To further examine the manufacturability and identify optimal sets, several (near) ideal SLP sets (Table 1) were synthesized using solid phase Fmoc/^{t}Bu chemistry, treated with a cleavage cocktail and analyzed by UPLC-MS.

### Material and Methods

All reagents and solvents for solid phase peptide synthesis were purchased from Merck, Sigma Aldrich, Actu-All, Bachem and Biosolve, GL Biochem and used as received.

### Solid Phase Peptide Synthesis (SPPS)

Peptides synthesis was performed on a Tetras peptide synthesizer (Advanced ChemTech) by solid phase Fmoc/^{t}Bu chemistry according to established methods. In general, the peptide synthesis was started from pre-loaded Wang-resin, pre-loaded HMPB ChemMatrix® resin, pre-loaded 2-chlorotrityl resin or pre-loaded 4-(1′,1′-dimethyl-1′-hydroxypropyl)phenoxyacetyl-alanyl-aminomethyl resin. The scale of the reaction was based on the type of resin used between 30 μmol and 60 μmol. The peptides were synthesized by single, double or triple coupling cycles or a combination of single, double and triple coupling cycles.

The initial swell procedure
1) Swelling of the resin: 2 cycles with NMP
2) NMP wash
3) iPrOH wash

A single coupling cycle was performed by the following consecutive steps:
1) Deprotection of the Fmoc-group: 3 cycles with piperidine in N-methyl pyrrolidone (NMP).
2) NMP wash
3) iPrOH wash
4) Coupling of the appropriate amino acid. After addition of the Fmoc-amino acid in NMP and the coupling reagent (3-(diethoxy-phosphoryloxy)-1,2,3-benzo[d]triazin-4(3H)-one (DEPBT), 2-(1H-benzotriazole-1-yl)-1,1,3,3-tetramethylaminium hexafluorophosphate (HBTU), 2-(1H-benzotriazol-1-yl)-N,N,N′,N′-tetramethylaminium tetrafluoroborate (TBTU), benzotriazole-1-yl-oxy-tris-pyrrolidino-phosphonium hexafluorophosphate (PyBOP), 7-aza-benzotriazol-1-yloxy-tripyrrolidinophosphonium hexafluorophosphate (PyAOP), Ethyl cyano(hydroxyimino)acetato-02-tri-(1-pyrrolidinyl)-phosphonium hexafluorophosphate (PyOxim), 1-[bis(dimethylamino)methylene]-1H-1,2,3-triazolo[4,5-b]pyridinium 3-oxid hexafluorophosphate (HATU), 1-[1-(Cyano-2-ethoxy-2-oxoethylideneaminooxy)-dimethylamino-morpholino]-uronium hexafluorophosphate (COMU), 2-(1-Oxy-pyridin-2-yl)-1,1,3,3-tetramethylisothiouronium tetrafluoroborate (TOTT), O-(1H-6-chlorobenzotriazole-1-yl)-1,1,3,3-tetramethyluronium hexafluorophosphate (HCTU), N,N′-diisopropylcarbodiimide (DIC) or ethyl 2-cyano-2-(hydroximino)acetate (Oxyma Pure®)) in NMP, the reaction mixture was shaken for 1 min. Optionally a base such as N-methylmorpholine (NMM) or N,N-diisopropylethylamine (DIPEA) was added. The reaction mixture was shaken for at least 30 min.
5) NMP wash
6) Capping by acetic anhydride or benzoyl chloride in the presence of a base (NMM or pyridine).
7) NMP wash
8) iPrOH wash

A double coupling cycle was performed by the following consecutive steps:
1) Deprotection of the Fmoc-group: 3 cycles with piperidine in NMP.
2) NMP wash
3) iPrOH wash
4) First coupling cycle: After addition of the Fmoc-amino acid in NMP and the coupling reagent (DEPBT, HBTU, TBTU, PyBOP, PyAOP, PyOxim HATU, COMU, TOTT, HCTU, DIC or Oxyma Pure®) in NMP, the reaction mixture was shaken for 1 min. Optionally a base (NMM or DIPEA) was added. The reaction mixture was shaken for a defined time of at least 15 min.
5) Purge of the reaction vessel.
6) Second coupling cycle: After addition of the Fmoc-amino acid in NMP and the coupling reagent (DEPBT, HBTU, TBTU, PyBOP, PyAOP, PyOxim, HATU, COMU, TOTT, HCTU, DIC or Oxyma Pure®)) in NMP, the reaction mixture was shaken for 1 min. Optionally a base (NMM or DIPEA) was added. The reaction mixture was shaken for a defined time of at least 15 min.
7) NMP wash
8) Capping by acetic anhydride or benzoyl chloride in the presence of a base (NMM or pyridine).
9) NMP wash
10) iPrOH wash

A triple coupling cycle was performed by the following consecutive steps:
1) Deprotection of the Fmoc-group: 3 cycles with piperidine in NMP.
2) NMP wash
3) iPrOH wash
4) First coupling cycle: After addition of the Fmoc-amino acid in NMP and the coupling reagent (DEPBT, HBTU, TBTU, PyBOP, PyAOP, PyOxim HATU, COMU, TOTT, HCTU, DIC or Oxyma Pure®) in NMP, the reaction mixture was shaken for 1 min. Optionally a base (NMM or DIPEA) was added. The reaction mixture was shaken for a defined time of at least 15 min.
5) Purge of the reaction vessel.
6) Second coupling cycle: After addition of the Fmoc-amino acid in NMP and the coupling reagent (DEPBT, HBTU, TBTU, PyBOP, PyAOP, PyOxim, HATU, COMU, TOTT, HCTU, DIC or Oxyma Pure®)) in NMP, the reaction mixture was shaken for 1 min. Optionally a base (NMM or DIPEA) was added. The reaction mixture was shaken for a defined time of at least 15 min.
7) Purge of the reaction vessel.
8) Third coupling cycle: After addition of the Fmoc-amino acid in NMP and the coupling reagent (DEPBT, HBTU, TBTU, PyBOP, PyAOP, PyOxim, HATU, COMU, TOTT, HCTU, DIC or Oxyma Pure®)) in NMP, the reaction mixture was shaken for 1 min. Optionally a base (NMM or DIPEA) was added. The reaction mixture was shaken for a defined time of at least 15 min.
9) NMP wash
10) Capping by acetic anhydride or benzoyl chloride in the presence of a base (NMM or pyridine).
11) NMP wash
12) iPrOH wash

After the last coupling cycle, the following final Fmoc-deprotection step and wash steps were performed:
1) Deprotection of the Fmoc-group: 3 cycles with piperidine in NMP.
2) NMP wash
3) iPrOH wash

Cleavage Procedure

After the final wash steps, the resin was dried, cooled and treated with a trifluoroacetic acid (TFA) based cleavage cocktail. A combination of scavengers such as H₂O, 1,2-ethanedithiol (EDT), triisopropylsilane (TIPS), 3,6-dioxa-1,8-octane-dithiol (DODT), ethanethiol (ET), triethylsilane (TES), phenol, thioanisole, 1-dodecanethiol, 1,4-dithioerythritol (DTE) or dithiothreitol (DTT) were added to the cleavage cocktail. After filtration of the resin, the reaction mixture was shaken at room temperature. Subsequently, the peptide was precipitated in an ether-based solution, centrifuged and the supernatant was removed. The solid precipitate was resuspended in an ether-based solution, centrifuged and the supernatant was removed. The resulting pellet was dissolved in a H₂O based mixture with acetonitrile (ACN) and TFA or with acetic acid and lyophilized overnight.

### Analysis of the Crude Peptide

The identity and purity of the crude peptides were determined by UPLC-MS on a Waters Acquity UPLC/TQD system using an C18 Waters Acquity BEH130 analytical column (1.7 um particle size, 2.1×150 mm, flow 0.4 mL/min) with a linear gradient (5% B to 95% B, linear gradient in 10 min). The absorbance was measured at 220 nm. The software MassLynx v4.1 was employed to integrate the individual peaks of the chromatogram in the region of the observed peptide-derived signals. The following ApexTrack peak detection parameters settings were employed for integration: peak-to-peak baseline noise was set at automatic, peak width at 5% height was set at automatic, baseline start threshold of 0.05% and baseline end threshold of 0.05%. The detect shoulder function was turned on. The following response thresholds were set: relative height of 0.20 and relative area of 0.10. After the peak of the desired peptide was identified by MS, the corresponding integral of the peak in the UV chromatogram is reported in Table 1 as percentage of the total integrated peaks, crude quality (%).

### Solvent System:

A: 0.1% formic acid (FA) and 1% ACN in H₂O

### B: 0.1% FA in ACN

### Results

Results of the peptide synthesis are shown in Table 1. The SLPs shown in this Table had been predicted to have good manufacturability and in most cases this prediction resulted into SLPs with a crude quality of at least 30% and generally higher than 40%. These qualities are high in respect to the number of couplings. A peptide of 30 amino acid residues synthesized with a coupling efficiency of 0.99 results into an overall yield of 74% (De Rosa L et al., (2013) Molecules 18: 440), a coupling efficiency of 0.96 results into an overall yield of 29%, a coupling efficiency of 0.93 results in an overall yield of only 11%, while the overall yield of a coupling efficiency of 0.90 is only 4.2%. The manufacturability of several prioritized SLPs having a high score was nevertheless difficult (SEQ ID NO:120-125).

The synthesis of SLPs for the region from position 19-51 of HPV-18 E6 appeared to be the most challenging part of this protein, even for 15-mers, as described previously by e.g. Atherton et. al (2018) Vaccine 36:2181. Crude quality for SEQ ID NO:1, 2 and 3, covering amino acids up to position 34 of HPV-18 E6 surprisingly turned out to be reasonable (25.9%) to good (>39.1%). Also, an SLP covering position 36-55 of HPV-18 E6 (SEQ ID NO 130) surprisingly turned out to have an acceptable crude quality of 26.4%, when analyzed using an ammonium acetate-based buffer system.

Similarly, the region from position 84-105 of HPV-18 E7 appeared to be challenging, as described by Atherton et al (2018) Vaccine 36:2181. Nevertheless, crude quality for SEQ ID NO 48, 50, 52, 54, 56, 58, 60, 63, covering the amino acid region from position 84 up to position 105 of HPV-18 E7 turned out to be surprisingly good (>39.9%).

For HPV-18, 52 of the 59 selected SLPs turned out to have a crude quality of at least >25% and the synthesis of 48 SLPs resulted into a crude quality of more than 30% of which 38 SLPs had a crude quality of even more than 40.0%. The latter group could be identified as highly potential candidates for a therapeutic vaccine towards HPV-18 patients. Based on these synthetic results of the individual SLPs, for every region of the E6 and E7 proteins, several sets of SLPs were identified in which every SLP turned out to have a suitable manufacturability.

For HPV-31, 24 of the 28 selected SLPs turned out to have a crude quality of at least >25% and the synthesis of 21 SLPs resulted into a crude quality of more than 30% of which 14 SLPs had a crude quality of even more than 40.0%. For every region, several SLPs were identified in which every SLP turned out to have a suitable manufacturability.

For HPV-33, 42 of the 50 selected SLPs turned out to have a crude quality of at least >25% and the synthesis of 41 SLPs resulted into a crude quality of more than 30% of which 35 SLPs had a crude quality of even more than 40.0%. For every region, several SLPs were identified in which every SLP turned out to have a suitable manufacturability.

For HPV-52, 31 of the 33 selected SLPs turned out to have a crude quality of at least >25% and the synthesis of 28 SLPs resulted into a crude quality of more than 30% of which 20 SLPs had a crude quality of even more than 40.0%. For every region, several SLPs were identified in which every SLP turned out to have a suitable manufacturability.

Similar results were obtained for HPV-45. Forty-seven of the 52 selected SLPs turned out to have a crude quality of at least >25% and the synthesis of 46 SLPs resulted into a crude quality of more than 30% of which 38 SLPs had a crude quality of even more than 40.0%. For every region, several SLPs were identified in which every SLP turned out to have a suitable manufacturability.

In conclusion, SLPs with high manufacturability were identified which have an overall coverage of the HPV-18 E6 and HPV-18 E7 combined, HPV-31 E6 and HPV-31 E7 combined, HPV-33 E6 and HPV-33 E7 combined, HPV-45 E6 and HPV-45 E7 combined or HPV-52 E6 and HPV-52 E7 combined proteins of more than 70% and are therefore highly potential candidates for a therapeutic vaccine towards HPV-18, HPV-31 HPV-33, HPV-45 or HPV-52 related cancers.

TABLE 1

| | | | | | Crude | SEQ |
|---|---|---|---|---|---|---|
| | Partial | Protein | | | quality | ID |
| Protein | region | region | SLP sequence | Score | (%) | NO |
| 18 E6 | 2-34 | 1-34 | ARFEDPTRRPYKLPDLCTE | 43.97 | 39.1 | 1 |
| | | | LNTSLQDIEITCVY | | | |
| 18 E6 | 1-34 | 1-34 | MARFEDPTRRPYKLPDLC | 41.39 | 25.9 | 2 |
| | | | TELNTSLQDIEITCVY | | | |
| 18 E6 | 3-34 | 1-34 | RFEDPTRRPYKLPDLCTEL | 43.18 | 56.2 | 3 |
| | | | NTSLQDIEITCVY | | | |
| 18 E6 | 43-77 | 43-140 | EVFEFAFKDLFVVYRDSIP | 38.25 | 39.4 | 4 |
| | | | HAACHKCIDFYSRIRE | | | |
| 18 E6 | 64-98 | 43-140 | ACHKCIDFYSRIRELRHYS | 42.96 | 45.7 | 5 |
| | | | DSVYGDTLEKLTNTGL | | | |
| 18 E6 | 85-119 | 43-140 | VYGDTLEKLTNTGLYNLLI | 45.62 | 55.4 | 6 |
| | | | RCLRCQKPLNPAEKLR | | | |
| 18 E6 | 106-140 | 43-140 | LRCQKPLNPAEKLRHLNE | 48.92 | 53.6 | 7 |
| | | | KRRFHNIAGHYRGQCHS | | | |
| 18 E6 | 43-77 | 43-140 | EVFEFAFKDLFVVYRDSIP | 38.25 | 39.4 | 8 |
| | | | HAACHKCIDFYSRIRE | | | |
| 18 E6 | 106-140 | 43-140 | LRCQKPLNPAEKLRHLNE | 48.92 | 53.6 | 9 |
| | | | KRRFHNIAGHYRGQCHS | | | |
| 18 E6 | 83-104 | 43-140 | DSVYGDTLEKLTNTGLYN | 49.01 | 65.5 | 10 |
| | | | LLIR | | | |
| 18 E6 | 73-96 | 43-140 | SRIRELRHYSDSVYGDTL | 49.47 | 54.9 | 11 |
| | | | EKLTNT | | | |
| 18 E6 | 91-119 | 43-140 | EKLTNTGLYNLLIRCLRCQ | 50.35 | 23.1 | 12 |
| | | | KPLNPAEKLR | | | |
| 18 E6 | 61-86 | 43-140 | PHAACHKCIDFYSRIRELR | 50.55 | 45.3 | 13 |
| | | | HYSDSVY | | | |
| 18 E6 | 50-71 | 43-140 | KDLFWYRDSIPHAACHK | 42.89 | 74.5 | 14 |
| | | | CIDF | | | |
| 18 E6 | 43-63 | 43-140 | EVFEFAFKDLFVVYRDSIP | 43.28 | 28.8 | 15 |
| | | | HA | | | |
| 18 E6 | 73-99 | 43-140 | SRIRELRHYSDSVYGDTL | 46.76 | 54.6 | 16 |
| | | | EKLTNTGLY | | | |
| 18 E6 | 86-119 | 43-140 | YGDTLEKLTNTGLYNLLIR | 47.58 | 33.2 | 17 |
| | | | CLRCQKPLNPAEKLR | | | |
| 18 E6 | 58-86 | 43-140 | DSIPHAACHKCIDFYSRIR | 48.29 | 60.9 | 18 |
| | | | ELRHYSDSVY | | | |
| 18 E6 | 106-140 | 43-140 | LRCQKPLNPAEKLRHLNE | 48.92 | 53.6 | 19 |
| | | | KRRFHNIAGHYRGQCHS | | | |
| 18 E6 | 43-63 | 43-140 | EVFEFAFKDLFVVYRDSIP | 43.28 | 28.8 | 20 |
| | | | HA | | | |
| 18 E6 | 64-97 | 43-140 | ACHKCIDFYSRIRELRHYS | 43.37 | 34.8 | 21 |
| | | | DSVYGDTLEKLTNTG | | | |
| 18 E6 | 50-78 | 43-140 | KDLFWYRDSIPHAACHK | 43.53 | 50.2 | 22 |
| | | | CIDFYSRIREL | | | |
| 18 E6 | 77-111 | 43-140 | ELRHYSDSVYGDTLEKLT | 46.79 | 52.7 | 23 |
| | | | NTGLYNLLIRCLRCQKP | | | |
| 18 E6 | 98-132 | 43-140 | LYNLLIRCLRCQKPLNPAE | 49.79 | 61.8 | 24 |
| | | | KLRHLNEKRRFHNIAG | | | |
| 18 E6 | 121-140 | 43-140 | LNEKRRFHNIAGHYRGQC | 52.43 | 69.9 | 25 |
| | | | HS | | | |
| 18 E6 | 43-69 | 43-140 | EVFEFAFKDLFVVYRDSIP | 30.15 | 29.5 | 26 |
| | | | HAACHKCI | | | |
| 18 E6 | 72-103 | 43-140 | YSRIRELRHYSDSVYGDT | 45.59 | 35.1 | 27 |
| | | | LEKLTNTGLYNLLI | | | |
| 18 E6 | 56-85 | 43-140 | YRDSIPHAACHKCIDFYS | 46.55 | 57.5 | 28 |
| | | | RIRELRHYSDSV | | | |
| 18 E6 | 107-140 | 43-140 | RCQKPLNPAEKLRHLNEK | 48.62 | 62.1 | 29 |
| | | | RRFHNIAGHYRGQCHS | | | |
| 18 E6 | 89-120 | 43-140 | TLEKLTNTGLYNLLIRCLR | 49.01 | 46.0 | 30 |
| | | | CQKPLNPAEKLRH | | | |
| 18 E6 | 139-158 | 139-158 | HSCCNRARQERLQRRRE | 62.18 | 72.7 | 31 |
| | | | TQV | | | |
| 18 E6 | 140-158 | 139-158 | SCCNRARQERLQRRRET | 64.36 | 46.4 | 32 |
| | | | QV | | | |
| 18 E7 | 1-20 | 1-62 | MHGPKATLQDIVLHLEPQ | 60.59 | 67.0 | 33 |
| | | | NE | | | |
| 18 E7 | 28-62 | 1-62 | HEQLSDSEEENDEIDGVN | 46.35 | 62.2 | 34 |
| | | | HQHLPARRAEPQRHTML | | | |
| 18 E7 | 7-41 | 1-62 | TLQDIVLHLEPQNEIPVDL | 48.88 | 53.9 | 35 |
| | | | LCHEQLSDSEEENDEI | | | |
| 18 E7 | 1-35 | 1-62 | MHGPKATLQDIVLHLEPQ | 44.58 | 67.0 | 36 |
| | | | NEIPVDLLCHEQLSDSE | | | |
| 18 E7 | 19-53 | 1-62 | NEIPVDLLCHEQLSDSEE | 51.16 | 59.1 | 37 |
| | | | ENDEIDGVNHQHLPARR | | | |
| 18 E7 | 28-62 | 1-62 | HEQLSDSEEENDEIDGVN | 46.35 | 62.2 | 38 |
| | | | HQHLPARRAEPQRHTML | | | |
| 18 E7 | 1-24 | 1-62 | MHGPKATLQDIVLHLEPQ | 55.29 | 44.7 | 39 |
| | | | NEIPVD | | | |
| 18 E7 | 11-42 | 1-62 | IVLHLEPQNEIPVDLLCHE | 49.91 | 50.4 | 40 |
| | | | QLSDSEEENDEID | | | |
| 18 E7 | 29-62 | 1-62 | EQLSDSEEENDEIDGVNH | 47.61 | 45.0 | 41 |
| | | | QHLPARRAEPQRHTML | | | |
| 18 E7 | 1-24 | 1-62 | MHGPKATLQDIVLHLEPQ | 55.29 | 44.7 | 42 |
| | | | NEIPVD | | | |
| 18 E7 | 11-45 | 1-62 | IVLHLEPQNEIPVDLLCHE | 50.35 | 45.0 | 43 |
| | | | QLSDSEEENDEIDGVN | | | |
| 18 E7 | 32-62 | 1-62 | SDSEEENDEIDGVNHQH | 48.02 | 38.9 | 44 |
| | | | LPARRAEPQRHTML | | | |
| 18 E7 | 1-28 | 1-62 | MHGPKATLQDIVLHLEPQ | 41.67 | 38.5 | 45 |
| | | | NEIPVDLLCH | | | |
| 18 E7 | 15-49 | 1-62 | LEPQNEIPVDLLCHEQLS | 50.59 | 56.0 | 46 |
| | | | DSEEENDEIDGVNHQHL | | | |
| 18 E7 | 36-62 | 1-62 | EENDEIDGVNHQHLPARR | 50.72 | 34.3 | 47 |
| | | | AEPQRHTML | | | |
| 18 E7 | 67-105 | 67-105 | KCEARIKLWESSADDLR | 27.20 | 42.6 | 48 |
| | | | AFQQLFLNTLSFVCPWCA | | | |
| | | | SQQ | | | |
| 18 E7 | 67-86 | 67-105 | KCEARIKLWESSADDLR | 55.10 | 27.6 | 49 |
| | | | AF | | | |
| 18 E7 | 73-105 | 67-105 | KLWESSADDLRAFQQLF | 30.43 | 39.9 | 50 |
| | | | LNTLSFVCPWCASQQ | | | |
| 18 E7 | 67-93 | 67-105 | KCEARIKLWESSADDLR | 37.76 | 63.4 | 51 |
| | | | AFQQLFLNT | | | |
| 18 E7 | 78-105 | 67-105 | SSADDLRAFQQLFLNTLS | 34.44 | 66.3 | 52 |
| | | | FVCPWCASQQ | | | |
| 18 E7 | 67-89 | 67-105 | KCEARIKLWESSADDLR | 54.29 | 29.2 | 53 |
| | | | AFQQL | | | |
| 18 E7 | 76-105 | 67-105 | VESSADDLRAFQQLFLNT | 33.01 | 53.2 | 54 |
| | | | LSFVCPWCASQQ | | | |
| 18 E7 | 67-91 | 67-105 | KCEARIKLWESSADDLR | 46.79 | 22.2 | 55 |
| | | | AFQQLFL | | | |
| 18 E7 | 78-105 | 67-105 | SSADDLRAFQQLFLNTLS | 34.44 | 66.3 | 56 |
| | | | FVCPWCASQQ | | | |
| 18 E7 | 67-94 | 67-105 | KCEARIKLWESSADDLR | 32.80 | 32.2 | 57 |
| | | | AFQQLFLNTL | | | |
| 18 E7 | 81-105 | 67-105 | DDLRAFQQLFLNTLSFVC | 36.16 | 54.8 | 58 |
| | | | PWCASQQ | | | |
| 18 E7 | 67-93 | 67-105 | KCEARIKLWESSADDLR | 37.76 | 63.4 | 59 |
| | | | AFQQLFLNT | | | |
| 18 E7 | 80-105 | 67-105 | ADDLRAFQQLFLNTLSFV | 34.52 | 44.3 | 60 |
| | | | CPWCASQQ | | | |
| 18 E7 | 67-86 | 67-105 | KCEARIKLWESSADDLR | 55.10 | 27.6 | 61 |
| | | | AF | | | |
| 18 E7 | 73-93 | 67-105 | KLWESSADDLRAFQQLF | 46.12 | 52.3 | 62 |
| | | | LNT | | | |
| 18 E7 | 80-105 | 67-105 | ADDLRAFQQLFLNTLSFV | 34.52 | 44.3 | 63 |
| | | | CPWCASQQ | | | |
| 45 E6 | 3-34 | 1-34 | RFDDPKQRPYKLPDLCTE | 41.68 | 48.6 | 64 |
| | | | LNTSLQDVSIACVY | | | |
| 45 E6 | 2-34 | 1-34 | ARFDDPKQRPYKLPDLCT | 37.31 | 32.6 | 65 |
| | | | ELNTSLQDVSIACVY | | | |
| 45 E6 | 7-34 | 1-34 | PKQRPYKLPDLCTELNTSL | 42.18 | 58.9 | 66 |
| | | | QDVSIACVY | | | |
| 45 E6 | 1-34 | 1-34 | MARFDDPKQRPYKLPDLC | 35.86 | 31.3 | 67 |
| | | | TELNTSLQDVSIACVY | | | |
| 45 E6 | 38-64 | 36-64 | TLERTEVYQFAFKDLCIVY | 29.32 | 34.9 | 68 |
| | | | RDCIAYAA | | | |
| 45 E6 | 40-64 | 36-64 | ERTEVYQFAFKDLCIVYRD | 34.24 | 34.3 | 69 |
| | | | CIAYAA | | | |
| 45 E6 | 36-64 | 36-64 | KATLERTEVYQFAFKDLCI | 30.87 | 35.4 | 70 |
| | | | VYRDCIAYAA | | | |
| 45 E6 | 37-64 | 36-64 | ATLERTEVYQFAFKDLCIV | 29.26 | 24.0 | 71 |
| | | | YRDCIAYAA | | | |
| 45 E6 | 117-140 | 60-140 | KRRHLKDKRRFHSIAGQY | 48.26 | 40.4 | 72 |
| | | | RGQCNT | | | |
| 45 E6 | 60-88 | 60-140 | IAYAACHKCIDFYSRIREL | 45.94 | 42.3 | 73 |
| | | | RYYSNSVYGE | | | |
| 45 E6 | 75-109 | 60-140 | IRELRYYSNSVYGETLEKI | 45.06 | 48.0 | 74 |
| | | | TNTELYNLLIRCLRCQ | | | |
| 45 E6 | 96-130 | 60-140 | TELYNLLIRCLRCQKPLNP | 45.34 | 57.8 | 75 |
| | | | AEKRRHLKDKRRFHSI | | | |
| 45 E6 | 117-140 | 60-140 | KRRHLKDKRRFHSIAGQY | 48.26 | 40.4 | 76 |
| | | | RGQCNT | | | |
| 45 E6 | 60-84 | 60-140 | IAYAACHKCIDFYSRIREL | 45.78 | 60.7 | 77 |
| | | | RYYSNS | | | |
| 45 E6 | 71-92 | 60-140 | FYSRIRELRYYSNSVYGET | 51.08 | 63.9 | 78 |
| | | | LEK | | | |
| 45 E6 | 79-106 | 60-140 | RYYSNSVYGETLEKITNTE | 48.94 | 43.7 | 79 |
| | | | LYNLLIRCL | | | |
| 45 E6 | 92-112 | 60-140 | KITNTELYNLLIRCLRCQK | 46.89 | 71.7 | 80 |
| | | | PL | | | |
| 45 E6 | 99-130 | 60-140 | YNLLIRCLRCQKPLNPAEK | 49.53 | 60.7 | 81 |
| | | | RRHLKDKRRFHSI | | | |
| 45 E6 | 109-140 | 60-140 | QKPLNPAEKRRHLKDKRR | 44.26 | 39.6 | 82 |
| | | | FHSIAGQYRGQCNT | | | |
| 45 E6 | 60-83 | 60-140 | IAYAACHKCIDFYSRIREL | 46.45 | 58.1 | 83 |
| | | | RYYSN | | | |
| 45 E6 | 70-101 | 60-140 | DFYSRIRELRYYSNSVYGE | 45.50 | 49.3 | 84 |
| | | | TLEKITNTELYNL | | | |
| 45 E6 | 86-119 | 60-140 | YGETLEKITNTELYNLLIR | 46.00 | 30.0 | 85 |
| | | | CLRCQKPLNPAEKRR | | | |
| 45 E6 | 106-140 | 60-140 | LRCQKPLNPAEKRRHLKD | 48.36 | 38.4 | 86 |
| | | | KRRFHSIAGQYRGQCNT | | | |
| 45 E6 | 60-88 | 60-140 | IAYAACHKCIDFYSRIREL | 45.94 | 42.3 | 87 |
| | | | RYYSNSVYGE | | | |
| 45 E6 | 75-99 | 60-140 | IRELRYYSNSVYGETLEKI | 44.54 | 58.1 | 88 |
| | | | TNTELY | | | |
| 45 E6 | 86-119 | 60-140 | YGETLEKITNTELYNLLIR | 46.00 | 30.0 | 89 |
| | | | CLRCQKPLNPAEKRR | | | |
| 45 E6 | 140-158 | 139-158 | TCCDQARQERLRRRRET | 63.79 | 50.6 | 90 |
| | | | QV | | | |
| 45 E7 | 1-30 | 1-65 | MHGPRETLQEIVLHLEPQ | 44.21 | 49.9 | 91 |
| | | | NELDPVDLLCYE | | | |
| 45 E7 | 17-45 | 1-65 | PQNELDPVDLLCYEQLSE | 51.46 | 57.9 | 92 |
| | | | SEEENDEADGV | | | |
| 45 E7 | 32-65 | 1-65 | LSESEEENDEADGVSHA | 49.85 | 53.6 | 93 |
| | | | QLPARRAEPQRHKILCV | | | |
| 45 E7 | 1-35 | 1-65 | MHGPRETLQEIVLHLEPQ | 41.10 | 42.9 | 94 |
| | | | NELDPVDLLCYEQLSES | | | |
| 45 E7 | 17-51 | 1-65 | PQNELDPVDLLCYEQLSE | 47.70 | 56.1 | 95 |
| | | | SEEENDEADGVSHAQLP | | | |
| 45 E7 | 32-65 | 1-65 | LSESEEENDEADGVSHA | 49.85 | 53.6 | 96 |
| | | | QLPARRAEPQRHKILCV | | | |
| 45 E7 | 1-26 | 1-65 | MHGPRETLQEIVLHLEPQ | 53.28 | 64.7 | 97 |
| | | | NELDPVDL | | | |
| 45 E7 | 13-47 | 1-65 | LHLEPQNELDPVDLLCYE | 47.99 | 63.4 | 98 |
| | | | QLSESEEENDEADGVSH | | | |
| 45 E7 | 34-65 | 1-65 | ESEEENDEADGVSHAQLP | 49.77 | 66.1 | 99 |
| | | | ARRAEPQRHKILCV | | | |
| 45 E7 | 1-26 | 1-65 | MHGPRETLQEIVLHLEPQ | 53.28 | 64.7 | 100 |
| | | | NELDPVDL | | | |
| 45 E7 | 13-36 | 1-65 | LHLEPQNELDPVDLLCYE | 50.93 | 66.3 | 101 |
| | | | QLSESE | | | |
| 45 E7 | 23-55 | 1-65 | PVDLLCYEQLSESEEEND | 51.40 | 57.3 | 102 |
| | | | EADGVSHAQLPARRA | | | |
| 45 E7 | 42-65 | 1-65 | ADGVSHAQLPARRAEPQ | 50.62 | 76.3 | 103 |
| | | | RHKILCV | | | |
| 45 E7 | 2-35 | 1-65 | HGPRETLQEIVLHLEPQN | 41.89 | 57.4 | 104 |
| | | | ELDPVDLLCYEQLSES | | | |
| 45 E7 | 36-65 | 1-65 | EEENDEADGVSHAQLPA | 50.93 | 64.7 | 105 |
| | | | RRAEPQRHKILCV | | | |
| 45 E7 | 1-26 | 1-65 | MHGPRETLQEIVLHLEPQ | 53.28 | 64.7 | 106 |
| | | | NELDPVDL | | | |
| 45 E7 | 13-35 | 1-65 | LHLEPQNELDPVDLLCYE | 51.25 | 73.2 | 107 |
| | | | QLSES | | | |
| 45 E7 | 22-49 | 1-65 | DPVDLLCYEQLSESEEEN | 52.60 | 49.5 | 108 |
| | | | DEADGVSHAQ | | | |
| 45 E7 | 36-65 | 1-65 | EEENDEADGVSHAQLPA | 50.93 | 64.7 | 109 |
| | | | RRAEPQRHKILCV | | | |
| 45 E7 | 74-106 | 68-106 | ELTVESSAEDLRTLQQLFL | 33.69 | 35.4 | 110 |
| | | | STLSFVCPWCATNQ | | | |
| 45 E7 | 68-87 | 68-106 | KCDGRIELTVESSAEDLR | 57.10 | 48.5 | 111 |
| | | | TL | | | |
| 45 E7 | 68-91 | 68-106 | KCDGRIELTVESSAEDLR | 47.76 | 59.5 | 112 |
| | | | TLQQLF | | | |
| 45 E7 | 78-97 | 68-106 | ESSAEDLRTLQQLFLSTLS | 51.29 | 52.6 | 113 |
| | | | F | | | |
| 45 E7 | 84-106 | 68-106 | LRTLQQLFLSTLSFVCPW | 34.78 | 60.6 | 114 |
| | | | CATNQ | | | |
| 45 E7 | 68-94 | 68-106 | KCDGRIELTVESSAEDLR | 40.62 | 40.2 | 115 |
| | | | TLQQLFLST | | | |
| 45 E7 | 81-100 | 68-106 | AEDLRTLQQLFLSTLSFVC | 42.34 | 49.9 | 116 |
| | | | P | | | |
| 45 E7 | 87-106 | 68-106 | LQQLFLSTLSFVCPWCAT | 40.94 | 23.3 | 117 |
| | | | NQ | | | |
| 45 E7 | 68-97 | 68-106 | KCDGRIELTVESSAEDLR | 33.78 | 43.3 | 118 |
| | | | TLQQLFLSTLSF | | | |
| 45 E7 | 84-106 | 68-106 | LRTLQQLFLSTLSFVCPW | 34.78 | 60.6 | 119 |
| | | | CATNQ | | | |
| 18 E6 | 43-74 | 43-140 | EVFEFAFKDLFVVYRDSIP | 36.14 | 16.4 | 120 |
| | | | HAACHKCIDFYSR | | | |
| 18 E6 | 101-135 | 43-140 | LLIRCLRCQKPLNPAEKLR | 48.98 | 10.3 | 121 |
| | | | HLNEKRRFHNIAGHYR | | | |
| 18 E7 | 22-56 | 20-56 | PVDLLCHEQLSDSEEEND | 48.01 | 19.8 | 122 |
| | | | EIDGVNHQHLPARRAEP | | | |
| 45 E6 | 101-135 | 60-140 | LLIRCLRCQKPLNPAEKRR | 50.55 | 10.9 | 123 |
| | | | HLKDKRRFHSIAGQYR | | | |
| 45 E6 | 60-94 | 60-140 | IAYAACHKCIDFYSRIREL | 36.51 | 14.1 | 124 |
| | | | RYYSNSVYGETLEKIT | | | |
| 45 E6 | 139-158 | 139-158 | NTCCDQARQERLRRRRET | 58.28 | 16.4 | 125 |
| | | | QV | | | |
| 18 E6 | 36-55 | 20-56 | KTVLELTEVFEFAFKDLFV | 41.55 | 26.4 | 130 |
| | | | V | | | |
| 18 E6 | 71-103 | 43-140 | FYSRIRELRHYSDSVYGD | 43.13 | 63.0 | 131 |
| | | | TLEKLTNTGLYNLLI | | | |
| 45 E7 | 17-40 | 1-65 | PQNELDPVDLLCYEQLSE | 49.51 | 73.3 | 132 |
| | | | SEEEND | | | |
| 45 E7 | 17-43 | 1-65 | PQNELDPVDLLCYEQLSE | 51.72 | 55.9 | 133 |
| | | | SEEENDEAD | | | |
| 33 E6 | 2-32 | 1-32 | FQDTEEKPRTLHDLCQAL | 47.96 | 50.1 | 136 |
| | | | ETTIHNIELQCVE | | | |
| 33 E6 | 1-32 | 1-32 | MFQDTEEKPRTLHDLCQA | 45.07 | 31.3 | 137 |
| | | | LETTIHNIELQCVE | | | |
| 33 E6 | 4-31 | 1-32 | DTEEKPRTLHDLCQALET | 51.25 | 54.7 | 138 |
| | | | TIHNIELQCV | | | |
| 33 E6 | 73-110 | 15-110 | ISEYRHYNYSVYGNTLEQ | 48.26 | 48.3 | 139 |
| | | | TVKKPLNEILIRCIICQRPL | | | |
| 33 E6 | 52-86 | 15-110 | VVYREGNPFGICKLCLRFL | 44.53 | 29.5 | 140 |
| | | | SKISEYRHYNYSVYGN | | | |
| 33 E6 | 19-47 | 15-110 | LETTIHNIELQCVECKKPL | 42.41 | 57.4 | 141 |
| | | | QRSEVYDFAF | | | |
| 33 E6 | 31-65 | 15-110 | VECKKPLQRSEVYDFAFA | 36.70 | 64.7 | 142 |
| | | | DLTVVYREGNPFGICKL | | | |
| 33 E6 | 18-47 | 15-110 | ALETTIHNIELQCVECKKP | 41.21 | 49.8 | 143 |
| | | | LQRSEVYDFAF | | | |
| 33 E6 | 34-65 | 15-110 | KKPLQRSEVYDFAFADLT | 35.79 | 55.8 | 144 |
| | | | VVYREGNPFGICKL | | | |
| 33 E6 | 34-68 | 15-110 | KKPLQRSEVYDFAFADLT | 37.26 | 8.9 | 145 |
| | | | WYREGNPFGICKLCLR | | | |
| 33 E6 | 55-89 | 15-110 | REGNPFGICKLCLRFLSKI | 46.37 | 17.8 | 146 |
| | | | SEYRHYNYSVYGNTLE | | | |
| 33 E6 | 76-110 | 15-110 | YRHYNYSVYGNTLEQTVK | 49.72 | 9.9 | 147 |
| | | | KPLNEILIRCIICQRPL | | | |
| 33 E6 | 15-44 | 15-110 | LCQALETTIHNIELQCVEC | 47.40 | 48.8 | 148 |
| | | | KKPLQRSEVYD | | | |
| 33 E6 | 58-92 | 15-110 | NPFGICKLCLRFLSKISEY | 45.04 | 52.5 | 149 |
| | | | RHYNYSVYGNTLEQTV | | | |
| 33 E6 | 77-110 | 15-110 | RHYNYSVYGNTLEQTVKK | 50.08 | 36.6 | 150 |
| | | | PLNEILIRCIICQRPL | | | |
| 33 E6 | 45-65 | 15-110 | FAFADLTWYREGNPFGIC | 44.51 | 58.1 | 151 |
| | | | KL | | | |
| 33 E6 | 61-95 | 15-110 | GICKLCLRFLSKISEYRHY | 41.74 | 45.1 | 152 |
| | | | NYSVYGNTLEQTVKKP | | | |
| 33 E6 | 19-44 | 15-110 | LETTIHNIELQCVECKKPL | 48.44 | 57.6 | 153 |
| | | | QRSEVYD | | | |
| 33 E6 | 82-110 | 15-110 | SVYGNTLEQTVKKPLNEIL | 41.88 | 13.0 | 154 |
| | | | IRCIICQRPL | | | |
| 33 E6 | 31-58 | 15-110 | VECKKPLQRSEVYDFAFA | 41.21 | 55.1 | 155 |
| | | | DLTWYREGN | | | |
| 33 E6 | 18-44 | 15-110 | ALETTIHNIELQCVECKKP | 48.41 | 38.7 | 156 |
| | | | LQRSEVYD | | | |
| 33 E6 | 56-90 | 15-110 | EGNPFGICKLCLRFLSKIS | 47.06 | 44.9 | 157 |
| | | | EYRHYNYSVYGNTLEQ | | | |
| 33 E6 | 89-110 | 15-110 | EQTVKKPLNEILIRCIICQ | 48.89 | 66.6 | 158 |
| | | | RPL | | | |
| 33 E6 | 73-102 | 15-110 | ISEYRHYNYSVYGNTLEQ | 46.90 | 10.9 | 159 |
| | | | TVKKPLNEILIR | | | |
| 33 E6 | 104-133 | 104-149 | IICQRPLCPQEKKRHVDL | 51.61 | 21.7 | 160 |
| | | | NKRFHNISGRWA | | | |
| 33 E6 | 120-149 | 104-149 | DLNKRFHNISGRWAGRC | 40.82 | 52.8 | 161 |
| | | | AACWRSRRRETAL | | | |
| 33 E6 | 115-149 | 104-149 | KKRHVDLNKRFHNISGR | 39.64 | 54.1 | 162 |
| | | | WAGRCAACWRSRRRETA | | | |
| | | | L | | | |
| 33 E6 | 104-128 | 104-149 | IICQRPLCPQEKKRHVDL | 55.45 | 57.7 | 163 |
| | | | NKRFHNI | | | |
| 33 E6 | 104-135 | 104-149 | IICQRPLCPQEKKRHVDL | 49.83 | 23.5 | 164 |
| | | | NKRFHNISGRWAGR | | | |
| 33 E6 | 122-149 | 104-149 | NKRFHNISGRWAGRCAA | 39.87 | 53.2 | 165 |
| | | | CWRSRRRETAL | | | |
| 33 E7 | 1-35 | 1-60 | MRGHKPTLKEYVLDLYPE | 41.86 | 54.1 | 166 |
| | | | PTDLYCYEQLSDSSDED | | | |
| 33 E7 | 26-60 | 1-60 | EQLSDSSDEDEGLDRPD | 43.23 | 66.0 | 167 |
| | | | GQAQPATADYYIVTCCHT | | | |
| 33 E7 | 22-41 | 1-60 | LYCYEQLSDSSDEDEGLD | 59.21 | 48.7 | 168 |
| | | | RP | | | |
| 33 E7 | 2-29 | 1-60 | RGHKPTLKEYVLDLYPEPT | 48.11 | 49.5 | 169 |
| | | | DLYCYEQLS | | | |
| 33 E7 | 5-39 | 1-60 | KPTLKEYVLDLYPEPTDLY | 42.49 | 59.9 | 170 |
| | | | CYEQLSDSSDEDEGLD | | | |
| 33 E7 | 33-60 | 1-60 | DEDEGLDRPDGQAQPAT | 43.93 | 68.0 | 171 |
| | | | ADYYIVTCCHT | | | |
| 33 E7 | 1-30 | 1-60 | MRGHKPTLKEYVLDLYPE | 45.36 | 52.3 | 172 |
| | | | PTDLYCYEQLSD | | | |
| 33 E7 | 17-46 | 1-60 | PEPTDLYCYEQLSDSSDE | 45.74 | 58.8 | 173 |
| | | | DEGLDRPDGQAQ | | | |
| 33 E7 | 21-50 | 1-60 | DLYCYEQLSDSSDEDEGL | 45.59 | 62.2 | 174 |
| | | | DRPDGQAQPATA | | | |
| 33 E7 | 37-60 | 1-60 | GLDRPDGQAQPATADYYI | 47.04 | 79.4 | 175 |
| | | | VTCCHT | | | |
| 33 E7 | 1-31 | 1-60 | MRGHKPTLKEYVLDLYPE | 44.42 | 23.5 | 176 |
| | | | PTDLYCYEQLSDS | | | |
| 33 E7 | 73-93 | 59-97 | ASDLRTIQQLLMGTVNIV | 43.72 | 44.1 | 177 |
| | | | CPT | | | |
| 33 E7 | 59-83 | 59-97 | HTCNTTVRLCVNSTASDL | 45.79 | 39.2 | 178 |
| | | | RTIQQLL | | | |
| 33 E7 | 73-97 | 59-97 | ASDLRTIQQLLMGTVNIV | 36.69 | 40.0 | 179 |
| | | | CPTCAQQ | | | |
| 33 E7 | 59-82 | 59-97 | HTCNTTVRLCVNSTASDL | 50.24 | 51.9 | 180 |
| | | | RTIQQL | | | |
| 33 E7 | 59-86 | 59-97 | HTCNTTVRLCVNSTASDL | 41.79 | 51.0 | 181 |
| | | | RTIQQLLMGT | | | |
| 33 E7 | 78-97 | 59-97 | TIQQLLMGTVNIVCPTCA | 44.01 | 36.6 | 182 |
| | | | QQ | | | |
| 33 E7 | 75-96 | 59-97 | DLRTIQQLLMGTVNIVCP | 39.66 | 39.5 | 183 |
| | | | TCAQ | | | |
| 33 E7 | 59-87 | 59-97 | HTCNTTVRLCVNSTASDL | 42.91 | 48.5 | 184 |
| | | | RTIQQLLMGTV | | | |
| 33 E7 | 47-67 | 47-67 | PATADYYIVTCCHTCNTT | 40.57 | 56.6 | 185 |
| | | | VRL | | | |
| 52 E6 | 1-32 | 1-32 | MFEDPATRPRTLHELCEV | 43.85 | 45.1 | 188 |
| | | | LEESVHEIRLQCVQ | | | |
| 52 E6 | 2-32 | 1-32 | FEDPATRPRTLHELCEVLE | 45.97 | 51.9 | 189 |
| | | | ESVHEIRLQCVQ | | | |
| 52 E6 | 5-32 | 1-32 | PATRPRTLHELCEVLEESV | 49.32 | 47.5 | 190 |
| | | | HEIRLQCVQ | | | |
| 52 E6 | 19-49 | 17-110 | LEESVHEIRLQCVQCKKE | 43.29 | 38.8 | 191 |
| | | | LQRREVYKFLFTD | | | |
| 52 E6 | 71-99 | 17-110 | SKISEYRHYQYSLYGKTLE | 46.80 | 68.8 | 192 |
| | | | ERVKKPLSEI | | | |
| 52 E6 | 61-82 | 17-110 | GVCIMCLRFLSKISEYRHY | 41.65 | 51.6 | 193 |
| | | | QYS | | | |
| 52 E6 | 82-110 | 17-110 | SLYGKTLEERVKKPLSEIT | 37.31 | 13.3 | 194 |
| | | | IRCIICQTPL | | | |
| 52 E6 | 49-73 | 17-110 | DLRIVYRDNNPYGVCIMC | 40.22 | 52.4 | 195 |
| | | | LRFLSKI | | | |
| 52 E6 | 40-62 | 17-110 | REVYKFLFTDLRIVYRDNN | 50.57 | 63.9 | 196 |
| | | | PYGV | | | |
| 52 E6 | 69-99 | 17-110 | FLSKISEYRHYQYSLYGKT | 43.59 | 28.8 | 197 |
| | | | LEERVKKPLSEI | | | |
| 52 E6 | 51-75 | 17-110 | RIVYRDNNPYGVCIMCLR | 42.05 | 17.6 | 198 |
| | | | FLSKISE | | | |
| 52 E6 | 18-47 | 17-110 | VLEESVHEIRLQCVQCKK | 46.93 | 44.1 | 199 |
| | | | ELQRREVYKFLF | | | |
| 52 E6 | 31-64 | 17-110 | VQCKKELQRREVYKFLFT | 45.77 | 32.6 | 200 |
| | | | DLRIVYRDNNPYGVCI | | | |
| 52 E6 | 89-110 | 17-110 | EERVKKPLSEITIRCIICQT | 47.83 | 49.6 | 201 |
| | | | PL | | | |
| 52 E6 | 72-101 | 17-110 | KISEYRHYQYSLYGKTLEE | 43.44 | 32.7 | 202 |
| | | | RVKKPLSEITI | | | |
| 52 E6 | 19-50 | 17-110 | LEESVHEIRLQCVQCKKE | 43.81 | 43.3 | 203 |
| | | | LQRREVYKFLFTDL | | | |
| 52 E6 | 60-84 | 17-110 | YGVCIMCLRFLSKISEYRH | 39.59 | 42.7 | 204 |
| | | | YQYSLY | | | |
| 52 E6 | 122-148 | 104-148 | NKRFHNIMGRWTGRCSE | 45.95 | 52.0 | 205 |
| | | | CWRPRPVTQV | | | |
| 52 E6 | 104-127 | 104-148 | IICQTPLCPEEKERHVNAN | 52.80 | 51.2 | 206 |
| | | | KRFHN | | | |
| 52 E6 | 120-148 | 104-148 | NANKRFHNIMGRWTGRC | 44.14 | 34.7 | 207 |
| | | | SECWRPRPVTQV | | | |
| 52 E6 | 114-148 | 104-148 | EKERHVNANKRFHNIMG | 41.93 | 28.0 | 208 |
| | | | RWTGRCSECWRPRPVTQ | | | |
| | | | V | | | |
| 52 E7 | 26-62 | 1-69 | EQLGDSSDEEDTDGVDR | 45.60 | 27.4 | 209 |
| | | | PDGQAEQATSNYYIVTYC | | | |
| | | | HS | | | |
| 52 E7 | 1-35 | 1-69 | MRGDKATIKDYILDLQPE | 45.29 | 40.2 | 210 |
| | | | TTDLHCYEQLGDSSDEE | | | |
| 52 E7 | 48-69 | 1-69 | EQATSNYYIVTYCHSCDS | 40.10 | 61.3 | 211 |
| | | | TLRL | | | |
| 52 E7 | 45-69 | 1-69 | GQAEQATSNYYIVTYCHS | 41.89 | 56.8 | 212 |
| | | | CDSTLRL | | | |
| 52 E7 | 22-51 | 1-69 | LHCYEQLGDSSDEEDTD | 45.48 | 61.0 | 213 |
| | | | GVDRPDGQAEQAT | | | |
| 52 E7 | 28-59 | 1-69 | LGDSSDEEDTDGVDRPD | 45.32 | 52.3 | 214 |
| | | | GQAEQATSNYYIVTY | | | |
| 52 E7 | 2-36 | 1-69 | RGDKATIKDYILDLQPETT | 47.38 | 39.4 | 215 |
| | | | DLHCYEQLGDSSDEED | | | |
| 52 E7 | 61-92 | 61-99 | HSCDSTLRLCIHSTATDL | 36.82 | 31.4 | 216 |
| | | | RTLQQMLLGTLQWV | | | |
| 52 E7 | 79-99 | 61-99 | RTLQQMLLGTLQVVCPGC | 42.86 | 51.8 | 217 |
| | | | ARL | | | |
| 52 E7 | 61-85 | 61-99 | HSCDSTLRLCIHSTATDL | 41.69 | 39.3 | 218 |
| | | | RTLQQML | | | |
| 52 E7 | 72-99 | 61-99 | HSTATDLRTLQQMLLGTL | 36.74 | 38.1 | 219 |
| | | | QWCPGCARL | | | |
| 52 E7 | 62-81 | 61-99 | SCDSTLRLCIHSTATDLRT | 46.96 | 54.4 | 220 |
| | | | L | | | |
| 31 E6 | 12-43 | 1-43 | LHELSSALEIPYDELRLNC | 42.53 | 52.5 | 223 |
| | | | VYCKGQLTETEVL | | | |
| 31 E6 | 2-32 | 1-43 | FKNPAERPRKLHELSSALE | 47.60 | 28.1 | 224 |
| | | | IPYDELRLNCVY | | | |
| 31 E6 | 1-25 | 1-43 | MFKNPAERPRKLHELSSA | 51.64 | 55.4 | 225 |
| | | | LEIPYDE | | | |
| 31 E6 | 19-43 | 1-43 | LEIPYDELRLNCVYCKGQL | 40.85 | 60.5 | 226 |
| | | | TETEVL | | | |
| 31 E6 | 37-62 | 30-63 | LTETEVLDFAFTDLTIVYR | 38.80 | 42.0 | 227 |
| | | | DDTPHGV | | | |
| 31 E6 | 60-94 | 50-105 | HGVCTKCLRFYSKVSEFR | 40.96 | 47.2 | 228 |
| | | | WYRYSVYGTTLEKLTNK | | | |
| 31 E6 | 53-73 | 50-105 | VYRDDTPHGVCTKCLRFY | 51.07 | 55.0 | 229 |
| | | | SKV | | | |
| 31 E6 | 78-102 | 50-105 | WYRYSVYGTTLEKLTNKG | 44.81 | 17.0 | 230 |
| | | | ICDLLIR | | | |
| 31 E6 | 50-84 | 50-105 | LTIVYRDDTPHGVCTKCL | 38.31 | 37.3 | 231 |
| | | | RFYSKVSEFRWYRYSVY | | | |
| 31 E6 | 71-105 | 50-105 | SKVSEFRWYRYSVYGTTL | 38.95 | 35.0 | 232 |
| | | | EKLTNKGICDLLIRCIT | | | |
| 31 E6 | 60-84 | 50-105 | HGVCTKCLRFYSKVSEFR | 43.23 | 49.2 | 233 |
| | | | WYRYSVY | | | |
| 31 E6 | 104-135 | 104-149 | ITCQRPLCPEEKQRHLDK | 44.26 | 39.2 | 234 |
| | | | KKRFHNIGGRWTGR | | | |
| 31 E6 | 120-149 | 104-149 | DKKKRFHNIGGRWTGRC | 43.02 | 34.3 | 235 |
| | | | IACWRRPRTETQV | | | |
| 31 E6 | 124-149 | 104-149 | RFHNIGGRWTGRCIACW | 45.34 | 47.2 | 236 |
| | | | RRPRTETQV | | | |
| 31 E6 | 104-128 | 104-149 | ITCQRPLCPEEKQRHLDK | 47.09 | 28.5 | 237 |
| | | | KKRFHNI | | | |
| 31 E7 | 32-60 | 1-60 | SDEEDVIDSPAGQAEPDT | 44.30 | 49.4 | 238 |
| | | | SNYNIVTFCCQ | | | |
| 31 E7 | 17-45 | 1-60 | PEATDLHCYEQLPDSSDE | 50.18 | 52.5 | 239 |
| | | | EDVIDSPAGQA | | | |
| 31 E7 | 2-30 | 1-60 | RGETPTLQDYVLDLQPEA | 46.60 | 57.9 | 240 |
| | | | TDLHCYEQLPD | | | |
| 31 E7 | 23-56 | 1-60 | HCYEQLPDSSDEEDVIDS | 49.24 | 22.1 | 241 |
| | | | PAGQAEPDTSNYNIVT | | | |
| 31 E7 | 1-35 | 1-60 | MRGETPTLQDYVLDLQPE | 45.69 | 61.2 | 242 |
| | | | ATDLHCYEQLPDSSDEE | | | |
| 31 E7 | 35-57 | 1-60 | EDVIDSPAGQAEPDTSNY | 48.17 | 82.2 | 243 |
| | | | NIVTF | | | |
| 31 E7 | 62-90 | 60-98 | KSTLRLCVQSTQVDIRILQ | 33.79 | 20.7 | 244 |
| | | | ELLMGSFGIV | | | |
| 31 E7 | 77-97 | 60-98 | RILQELLMGSFGIVCPNCS | 41.09 | 53.9 | 245 |
| | | | TR | | | |
| 31 E7 | 62-98 | 60-98 | KSTLRLCVQSTQVDIRILQ | 34.33 | 28.9 | 246 |
| | | | ELLMGSFGIVCPNCSTRL | | | |
| 31 E7 | 69-90 | 60-98 | VQSTQVDIRILQELLMGS | 37.63 | 35.0 | 247 |
| | | | FGIV | | | |
| 31 E7 | 62-82 | 60-98 | KSTLRLCVQSTQVDIRILQ | 50.48 | 19.6 | 248 |
| | | | EL | | | |
| 31 E7 | 69-93 | 60-98 | VQSTQVDIRILQELLMGS | 32.62 | 35.0 | 249 |
| | | | FGIVCPN | | | |
| 31 E7 | 62-93 | 60-98 | KSTLRLCVQSTQVDIRILQ | 32.73 | 38.0 | 250 |
| | | | ELLMGSFGIVCPN | | | |

1. A method for treating an infection, disorder or disease caused by a human papillomavirus (HPV) type other than HPV-16, comprising the steps of:
a) providing a sample from a human subject suspected of having cells expressing HPV genes,
b) subjecting the sample to an assay to determine whether said human subject has cells expressing HPV genes and, if so, of which HPV type,
c) providing, if the subject has cells expressing HPV genes and the type is not HPV-16, one or more immunogenic composition(s) comprising a plurality of peptides, wherein each of said peptides comprises or consists of a contiguous fragment of 18-95 amino acids in length of the sequence of the E6 or the E7 protein of the HPV type identified in the sample, wherein the contiguous E6 and E7 fragments comprised within the plurality of peptides together in total cover at least 70% of the combined E6 and E7 sequences, and
d) treating said human subject with said immunogenic composition(s) and/or treating said human subject with a population of antigen-loaded activated antigen presenting cells (APCs) or a population of expanded antigen-specific T cells, wherein said population of cells has been generated ex vivo using said immunogenic composition(s).

2. The method according to claim 1, wherein at least one peptide or at least one immunogenic composition provided in step c) is manufactured after the determination of the type in step b).

3. The method according to claim 1, wherein said HPV other than HPV-16 is HPV-18 and wherein said composition(s) comprise(s):
i) the peptide set forth in SEQ ID NO:1; or
ii) the peptide set forth in SEQ ID NO:2; or
iii) the peptide set forth in SEQ ID NO:3.

4. The method according to claim 1, wherein said HPV other than HPV-16 is HPV-18 and wherein said composition(s) comprise(s) the peptide set forth in SEQ ID NO:130.

5. The method according to claim 1, wherein said HPV other than HPV-16 is HPV-18 and wherein said composition(s) comprise(s):
i) one, two, three or all of the peptides set forth in SEQ ID NO:4, 5, 6 and 7; or
ii) one, two, three, four, five or all of the peptides set forth in SEQ ID NO:8, 9, 10, 11, 12 and 13; or
iii) one, two, three, four, five or all of the peptides set forth in SEQ ID NO:14, 15, 16, 17, 18 and 19; or
iv) one, two, three, four, five or all of the peptides set forth in SEQ ID NO:20, 21, 22, 23, 24 and 25; or
v) one, two, three, four or all of the peptides set forth in SEQ ID NO:26, 27, 28, 29 and 30, or
vi) one, two, three, four or all of the peptides set forth in SEQ ID NO:26, 131, 28, 29 and 30.

6. The method according to claim 1, wherein said HPV other than HPV-16 is HPV-18 and wherein said composition(s) comprise(s):
i) the peptide set forth in SEQ ID NO:31; or
ii) the peptide set forth in SEQ ID NO:32.

7. The method according to claim 1, wherein said HPV other than HPV-16 is HPV-18 and wherein said composition(s) comprise(s):
i) one, two or all of the peptides set forth in SEQ ID NO:33, 34 and 35; or
ii) one, two or all of the peptides set forth in SEQ ID NO:36, 37 and 38; or
iii) one, two or all of the peptides set forth in SEQ ID NO:39, 40 and 41; or
iv) one, two or all of the peptides set forth in SEQ ID NO:42, 43 and 44; or
v) one, two or all of the peptides set forth in SEQ ID NO:45, 46 and 47.

8. The method according to claim 1, wherein said HPV other than HPV-16 is HPV-18 and wherein said composition(s) comprise(s):
i) the peptide set forth in SEQ ID NO:48; or
ii) one or both of the peptides set forth in SEQ ID NO:49 and 50; or
iii) one or both of the peptides set forth in SEQ ID NO:51 and 52; or
iv) one or both of the peptides set forth in SEQ ID NO:53 and 54; or
v) one or both of the peptides set forth in SEQ ID NO:55 and 56; or
vi) one or both of the peptides set forth in SEQ ID NO:57 and 58; or
vii) one or both of the peptides set forth in SEQ ID NO:59 and 60; or
viii) one, two or all of the peptides set forth in SEQ ID NO:61, 62 and 63.

9. The method according to claim 1, wherein said HPV other than HPV-16 is HPV-45 and wherein said composition(s) comprise(s):
i) the peptide set forth in SEQ ID NO:64; or
ii) the peptide set forth in SEQ ID NO:65; or
iii) the peptide set forth in SEQ ID NO:66 or
iv) the peptide set forth in SEQ ID NO:67.

10. The method according to claim 1, wherein said HPV other than HPV-16 is HPV-45 and wherein said composition(s) comprise(s):
i) the peptide set forth in SEQ ID NO:68; or
ii) the peptide set forth in SEQ ID NO:69; or
iii) the peptide set forth in SEQ ID NO:70; or
iv) the peptide set forth in SEQ ID NO:71.

11. The method according to claim 1, wherein said HPV other than HPV-16 is HPV-45 and wherein said composition(s) comprise(s):
i) one, two, three or all of the peptides set forth in SEQ ID NO:72, 73, 74 and 75; or
ii) one, two, three, four, five or all of the peptides set forth in SEQ ID NO:76, 77, 78, 79, 80 and 81; or
iii) one, two, three, four or all of the peptides set forth in SEQ ID NO:82, 83, 84 and 85; or
iv) one, two, three or all of the peptides set forth in SEQ ID NO:86, 87, 88 and 89.

12. The method according to claim 1,
wherein said HPV other than HPV-16 is HPV-45 and wherein said composition(s) comprise(s) the peptide set forth in SEQ ID NO:90.

13. The method according to claim 1, wherein said HPV other than HPV-16 is HPV-45 and wherein said composition(s) comprise(s):
i) one, two or all of the peptides set forth in SEQ ID NO:91, 92 and 93; or
ii) one, two or all of the peptides set forth in SEQ ID NO:94, 95 and 96; or
iii) one, two or all of the peptides set forth in SEQ ID NO:97, 98 and 99; or
iv) one, two, three or all of the peptides set forth in SEQ ID NO:100, 101, 102 and 103;
v) one or both of the peptides set forth in SEQ ID NO:104 and 105, or
vi) one, two, three or all of the peptides set forth in SEQ ID NO:106, 107, 108 and 109, or
vii) one, two or all of the peptides set forth in SEQ ID NO: 93, 104 and 132, or
viii) one, two or all of the peptides set forth in SEQ ID NO: 93, 104 and 133.

14. The method according to claim 1, wherein said HPV other than HPV-16 is HPV-45 and wherein said composition(s) comprise(s):
i) one or both of the peptides set forth in SEQ ID NO:110 and 111; or
ii) one, two or all of the peptides set forth in SEQ ID NO:112, 113 and 114; or
iii) one, two or all of the peptides set forth in SEQ ID NO:115, 116 and 117, or
iv) one or both of the peptides set forth in SEQ ID NO:118 and 119.

15. The method according to claim 1, wherein said HPV other than HPV-16 is HPV-33 and wherein said composition(s) comprise(s):
i) the peptide set forth in SEQ ID NO:136; or
ii) the peptide set forth in SEQ ID NO:137; or
iii) the peptide set forth in SEQ ID NO:138.

16. The method according to claim 1, wherein said HPV other than HPV-16 is HPV-33 and wherein said composition(s) comprise(s):
i) one, two, three or all of the peptides set forth in SEQ ID NO:139, 140, 141 and 142; or
ii) one, two, three or all of the peptides set forth in SEQ ID NO:139, 140, 143 and 144; or
iii) one, two, three or all of the peptides set forth in SEQ ID NO:141, 145, 146 and 147; or
iv) one, two, three or all of the peptides set forth in SEQ ID NO:145, 148, 149 and 150; or
v) one, two, three, four or all of the peptides set forth in SEQ ID NO:151, 152, 153, 154 and 155; or
vi) one, two, three, four or all of the peptides set forth in SEQ ID NO:142, 156, 157, 158 and 159.

17.-20. (canceled)

21. The method according to claim 1, wherein said HPV other than HPV-16 is HPV-52 and wherein said composition(s) comprise(s):
i) the peptide set forth in SEQ ID NO:188; or
ii) the peptide set forth in SEQ ID NO:189; or
iii) the peptide set forth in SEQ ID NO:190.

22.-25. (canceled)

26. The method according to claim 1, wherein said HPV other than HPV-16 is HPV-31 and wherein said composition(s) comprise(s):
i) one or both of the peptides set forth in SEQ ID NO:223 and 224; or
ii) one or both of the peptides set forth in SEQ ID NO:225 and 226; or
iii) one or both of the peptides set forth in SEQ ID NO:224 and 226.

27.-36. (canceled)

37. An immunogenic composition comprising one or more peptides selected from the group of peptides set forth in SEQ ID NO:1, 2, 3, 130, 48, 50, 52, 54, 56, 58, 60 and 63.

38. A method for treating an infection, disorder or disease caused by HPV-18, comprising administration to a subject of one or more peptides selected from the group of peptides set forth in: SEQ ID NO:1, 2, 3, 130, 48, 50, 52, 54, 56, 58, 60 and 63.

39.-40. (canceled)


SEQUENCE LISTING (250 sequences)

SEQ ID NO: 1 - protein, 33 aa, Human papillomavirus type 18
ARFEDPTRRPYKLPDLCTELNTSLQDIEITCVY

SEQ ID NO: 2 - protein, 34 aa, Human papillomavirus type 18
MARFEDPTRRPYKLPDLCTELNTSLQDIEITCVY

SEQ ID NO: 3 - protein, 32 aa, Human papillomavirus type 18
RFEDPTRRPYKLPDLCTELNTSLQDIEITCVY

SEQ ID NO: 4 - protein, 35 aa, Human papillomavirus type 18
EVFEFAFKDLFVVYRDSIPHAACHKCIDFYSRIRE

SEQ ID NO: 5 - protein, 35 aa, Human papillomavirus type 18
ACHKCIDFYSRIRELRHYSDSVYGDTLEKLTNTGL

SEQ ID NO: 6 - protein, 35 aa, Human papillomavirus type 18
VYGDTLEKLTNTGLYNLLIRCLRCQKPLNPAEKLR

SEQ ID NO: 7 - protein, 35 aa, Human papillomavirus type 18
LRCQKPLNPAEKLRHLNEKRRFHNIAGHYRGQCHS

SEQ ID NO: 8 - protein, 35 aa, Human papillomavirus type 18; identical to SEQ ID NO: 4

SEQ ID NO: 9 - protein, 35 aa, Human papillomavirus type 18; identical to SEQ ID NO: 7

SEQ ID NO: 10 - protein, 22 aa, Human papillomavirus type 18
DSVYGDTLEKLTNTGLYNLLIR

SEQ ID NO: 11 - protein, 24 aa, Human papillomavirus type 18
SRIRELRHYSDSVYGDTLEKLTNT

SEQ ID NO: 12 - protein, 29 aa, Human papillomavirus type 18
EKLTNTGLYNLLIRCLRCQKPLNPAEKLR

SEQ ID NO: 13 - protein, 26 aa, Human papillomavirus type 18
PHAACHKCIDFYSRIRELRHYSDSVY

SEQ ID NO: 14 - protein, 22 aa, Human papillomavirus type 18
KDLFVVYRDSIPHAACHKCIDF

SEQ ID NO: 15 - protein, 21 aa, Human papillomavirus type 18
EVFEFAFKDLFVVYRDSIPHA

SEQ ID NO: 16 - protein, 27 aa, Human papillomavirus type 18
SRIRELRHYSDSVYGDTLEKLTNTGLY

SEQ ID NO: 17 - protein, 34 aa, Human papillomavirus type 18
YGDTLEKLTNTGLYNLLIRCLRCQKPLNPAEKLR

SEQ ID NO: 18 - protein, 29 aa, Human papillomavirus type 18
DSIPHAACHKCIDFYSRIRELRHYSDSVY

SEQ ID NO: 19 - protein, 35 aa, Human papillomavirus type 18; identical to SEQ ID NO: 7

SEQ ID NO: 20 - protein, 21 aa, Human papillomavirus type 18
EVFEFAFKDLFVVYRDSIPHA

SEQ ID NO: 21 - protein, 34 aa, Human papillomavirus type 18
ACHKCIDFYSRIRELRHYSDSVYGDTLEKLTNTG

SEQ ID NO: 22 - protein, 29 aa, Human papillomavirus type 18
KDLFVVYRDSIPHAACHKCIDFYSRIREL

SEQ ID NO: 23 - protein, 35 aa, Human papillomavirus type 18
ELRHYSDSVYGDTLEKLTNTGLYNLLIRCLRCQKP

SEQ ID NO: 24 - protein, 35 aa, Human papillomavirus type 18
LYNLLIRCLRCQKPLNPAEKLRHLNEKRRFHNIAG

SEQ ID NO: 25 - protein, 20 aa, Human papillomavirus type 18
LNEKRRFHNIAGHYRGQCHS

SEQ ID NO: 26 - protein, 27 aa, Human papillomavirus type 18
EVFEFAFKDLFVVYRDSIPHAACHKCI

SEQ ID NO: 27 - protein, 32 aa, Human papillomavirus type 18
YSRIRELRHYSDSVYGDTLEKLTNTGLYNLLI

SEQ ID NO: 28 - protein, 30 aa, Human papillomavirus type 18
YRDSIPHAACHKCIDFYSRIRELRHYSDSV

SEQ ID NO: 29 - protein, 34 aa, Human papillomavirus type 18
RCQKPLNPAEKLRHLNEKRRFHNIAGHYRGQCHS

SEQ ID NO: 30 - protein, 32 aa, Human papillomavirus type 18
TLEKLTNTGLYNLLIRCLRCQKPLNPAEKLRH

SEQ ID NO: 31 - protein, 20 aa, Human papillomavirus type 18
HSCCNRARQERLQRRRETQV

SEQ ID NO: 32 - protein, 19 aa, Human papillomavirus type 18
SCCNRARQERLQRRRETQV

SEQ ID NO: 33 - protein, 20 aa, Human papillomavirus type 18
MHGPKATLQDIVLHLEPQNE

SEQ ID NO: 34 - protein, 35 aa, Human papillomavirus type 18
HEQLSDSEEENDEIDGVNHQHLPARRAEPQRHTML

SEQ ID NO: 35 - protein, 35 aa, Human papillomavirus type 18
TLQDIVLHLEPQNEIPVDLLCHEQLSDSEEENDEI

SEQ ID NO: 36 - protein, 35 aa, Human papillomavirus type 18
MHGPKATLQDIVLHLEPQNEIPVDLLCHEQLSDSE

SEQ ID NO: 37 - protein, 35 aa, Human papillomavirus type 18
NEIPVDLLCHEQLSDSEEENDEIDGVNHQHLPARR

SEQ ID NO: 38 - protein, 35 aa, Human papillomavirus type 18; identical to SEQ ID NO: 34

SEQ ID NO: 39 - protein, 24 aa, Human papillomavirus type 18
MHGPKATLQDIVLHLEPQNEIPVD

SEQ ID NO: 40 - protein, 32 aa, Human papillomavirus type 18
IVLHLEPQNEIPVDLLCHEQLSDSEEENDEID

SEQ ID NO: 41 - protein, 34 aa, Human papillomavirus type 18
EQLSDSEEENDEIDGVNHQHLPARRAEPQRHTML

SEQ ID NO: 42 - protein, 24 aa, Human papillomavirus type 18
MHGPKATLQDIVLHLEPQNEIPVD

SEQ ID NO: 43 - protein, 35 aa, Human papillomavirus type 18
IVLHLEPQNEIPVDLLCHEQLSDSEEENDEIDGVN

SEQ ID NO: 44 - protein, 31 aa, Human papillomavirus type 18
SDSEEENDEIDGVNHQHLPARRAEPQRHTML

SEQ ID NO: 45 - protein, 28 aa, Human papillomavirus type 18
MHGPKATLQDIVLHLEPQNEIPVDLLCH

SEQ ID NO: 46 - protein, 35 aa, Human papillomavirus type 18
LEPQNEIPVDLLCHEQLSDSEEENDEIDGVNHQHL

SEQ ID NO: 47 - protein, 27 aa, Human papillomavirus type 18
EENDEIDGVNHQHLPARRAEPQRHTML

SEQ ID NO: 48 - protein, 39 aa, Human papillomavirus type 18
KCEARIKLVVESSADDLRAFQQLFLNTLSFVCPWCASQQ

SEQ ID NO: 49 - protein, 20 aa, Human papillomavirus type 18
KCEARIKLVVESSADDLRAF

SEQ ID NO: 50 - protein, 33 aa, Human papillomavirus type 18
KLVVESSADDLRAFQQLFLNTLSFVCPWCASQQ

SEQ ID NO: 51 - protein, 27 aa, Human papillomavirus type 18
KCEARIKLVVESSADDLRAFQQLFLNT

SEQ ID NO: 52 - protein, 28 aa, Human papillomavirus type 18
SSADDLRAFQQLFLNTLSFVCPWCASQQ

SEQ ID NO: 53 - protein, 23 aa, Human papillomavirus type 18
KCEARIKLVVESSADDLRAFQQL

SEQ ID NO: 54 - protein, 30 aa, Human papillomavirus type 18
VESSADDLRAFQQLFLNTLSFVCPWCASQQ

SEQ ID NO: 55 - protein, 25 aa, Human papillomavirus type 18
KCEARIKLVVESSADDLRAFQQLFL

SEQ ID NO: 56 - protein, 28 aa, Human papillomavirus type 18
SSADDLRAFQQLFLNTLSFVCPWCASQQ

SEQ ID NO: 57 - protein, 28 aa, Human papillomavirus type 18
KCEARIKLVVESSADDLRAFQQLFLNTL

SEQ ID NO: 58 - protein, 25 aa, Human papillomavirus type 18
DDLRAFQQLFLNTLSFVCPWCASQQ

SEQ ID NO: 59 - protein, 27 aa, Human papillomavirus type 18
KCEARIKLVVESSADDLRAFQQLFLNT

SEQ ID NO: 60 - protein, 26 aa, Human papillomavirus type 18
ADDLRAFQQLFLNTLSFVCPWCASQQ

SEQ ID NO: 61 - protein, 20 aa, Human papillomavirus type 18
KCEARIKLVVESSADDLRAF

SEQ ID NO: 62 - protein, 21 aa, Human papillomavirus type 18
KLVVESSADDLRAFQQLFLNT

SEQ ID NO: 63 - protein, 26 aa, Human papillomavirus type 18
ADDLRAFQQLFLNTLSFVCPWCASQQ

SEQ ID NO: 64 - protein, 32 aa, Human papillomavirus type 45
RFDDPKQRPYKLPDLCTELNTSLQDVSIACVY

SEQ ID NO: 65 - protein, 33 aa, Human papillomavirus type 45
ARFDDPKQRPYKLPDLCTELNTSLQDVSIACVY

SEQ ID NO: 66 - protein, 28 aa, Human papillomavirus type 45
PKQRPYKLPDLCTELNTSLQDVSIACVY

SEQ ID NO: 67 - protein, 34 aa, Human papillomavirus type 45
MARFDDPKQRPYKLPDLCTELNTSLQDVSIACVY

SEQ ID NO: 68 - protein, 27 aa, Human papillomavirus type 45
TLERTEVYQFAFKDLCIVYRDCIAYAA

SEQ ID NO: 69 - protein, 25 aa, Human papillomavirus type 45
ERTEVYQFAFKDLCIVYRDCIAYAA

SEQ ID NO: 70 - protein, 29 aa, Human papillomavirus type 45
KATLERTEVYQFAFKDLCIVYRDCIAYAA

SEQ ID NO: 71 - protein, 28 aa, Human papillomavirus type 45
ATLERTEVYQFAFKDLCIVYRDCIAYAA

SEQ ID NO: 72 - protein, 24 aa, Human papillomavirus type 45
KRRHLKDKRRFHSIAGQYRGQCNT

SEQ ID NO: 73 - protein, 29 aa, Human papillomavirus type 45
IAYAACHKCIDFYSRIRELRYYSNSVYGE

SEQ ID NO: 74 - protein, 35 aa, Human papillomavirus type 45
IRELRYYSNSVYGETLEKITNTELYNLLIRCLRCQ

SEQ ID NO: 75 - protein, 35 aa, Human papillomavirus type 45
TELYNLLIRCLRCQKPLNPAEKRRHLKDKRRFHSI

SEQ ID NO: 76 - protein, 24 aa, Human papillomavirus type 45
KRRHLKDKRRFHSIAGQYRGQCNT

SEQ ID NO: 77 - protein, 25 aa, Human papillomavirus type 45
IAYAACHKCIDFYSRIRELRYYSNS

SEQ ID NO: 78 - protein, 22 aa, Human papillomavirus type 45
FYSRIRELRYYSNSVYGETLEK

SEQ ID NO: 79 - protein, 28 aa, Human papillomavirus type 45
RYYSNSVYGETLEKITNTELYNLLIRCL

SEQ ID NO: 80 - protein, 21 aa, Human papillomavirus type 45
KITNTELYNLLIRCLRCQKPL

SEQ ID NO: 81 - protein, 32 aa, Human papillomavirus type 45
YNLLIRCLRCQKPLNPAEKRRHLKDKRRFHSI

SEQ ID NO: 82 - protein, 32 aa, Human papillomavirus type 45
QKPLNPAEKRRHLKDKRRFHSIAGQYRGQCNT

SEQ ID NO: 83 - protein, 24 aa, Human papillomavirus type 45
IAYAACHKCIDFYSRIRELRYYSN

SEQ ID NO: 84 - protein, 32 aa, Human papillomavirus type 45
DFYSRIRELRYYSNSVYGETLEKITNTELYNL

SEQ ID NO: 85 - protein, 34 aa, Human papillomavirus type 45
YGETLEKITNTELYNLLIRCLRCQKPLNPAEKRR

SEQ ID NO: 86 - protein, 35 aa, Human papillomavirus type 45
LRCQKPLNPAEKRRHLKDKRRFHSIAGQYRGQCNT

SEQ ID NO: 87 - protein, 29 aa, Human papillomavirus type 45
IAYAACHKCIDFYSRIRELRYYSNSVYGE

SEQ ID NO: 88 - protein, 25 aa, Human papillomavirus type 45
IRELRYYSNSVYGETLEKITNTELY

SEQ ID NO: 89 - protein, 34 aa, Human papillomavirus type 45; identical to SEQ ID NO: 85

SEQ ID NO: 90 - protein, 19 aa, Human papillomavirus type 45
TCCDQARQERLRRRRETQV

SEQ ID NO: 91 - protein, 30 aa, Human papillomavirus type 45
MHGPRETLQEIVLHLEPQNELDPVDLLCYE

SEQ ID NO: 92 - protein, 29 aa, Human papillomavirus type 45
PQNELDPVDLLCYEQLSESEEENDEADGV

SEQ ID NO: 93 - protein, 34 aa, Human papillomavirus type 45
LSESEEENDEADGVSHAQLPARRAEPQRHKILCV

SEQ ID NO: 94 - protein, 35 aa, Human papillomavirus type 45
MHGPRETLQEIVLHLEPQNELDPVDLLCYEQLSES

SEQ ID NO: 95 - protein, 35 aa, Human papillomavirus type 45
PQNELDPVDLLCYEQLSESEEENDEADGVSHAQLP

SEQ ID NO: 96 - protein, 34 aa, Human papillomavirus type 45; identical to SEQ ID NO: 93

SEQ ID NO: 97 - protein, 26 aa, Human papillomavirus type 45
MHGPRETLQEIVLHLEPQNELDPVDL

SEQ ID NO: 98 - protein, 35 aa, Human papillomavirus type 45
LHLEPQNELDPVDLLCYEQLSESEEENDEADGVSH

SEQ ID NO: 99 - protein, 32 aa, Human papillomavirus type 45
ESEEENDEADGVSHAQLPARRAEPQRHKILCV

SEQ ID NO: 100 - protein, 26 aa, Human papillomavirus type 45
MHGPRETLQEIVLHLEPQNELDPVDL

SEQ ID NO: 101 - protein, 24 aa, Human papillomavirus type 45
LHLEPQNELDPVDLLCYEQLSESE

SEQ ID NO: 102 - protein, 33 aa, Human papillomavirus type 45
PVDLLCYEQLSESEEENDEADGVSHAQLPARRA

SEQ ID NO: 103 - protein, 24 aa, Human papillomavirus type 45
ADGVSHAQLPARRAEPQRHKILCV

SEQ ID NO: 104 - protein, 34 aa, Human papillomavirus type 45
HGPRETLQEIVLHLEPQNELDPVDLLCYEQLSES

SEQ ID NO: 105 - protein, 30 aa, Human papillomavirus type 45
EEENDEADGVSHAQLPARRAEPQRHKILCV

SEQ ID NO: 106 - protein, 26 aa, Human papillomavirus type 45
MHGPRETLQEIVLHLEPQNELDPVDL

SEQ ID NO: 107 - protein, 23 aa, Human papillomavirus type 45
LHLEPQNELDPVDLLCYEQLSES

SEQ ID NO: 108 - protein, 28 aa, Human papillomavirus type 45
DPVDLLCYEQLSESEEENDEADGVSHAQ

SEQ ID NO: 109 - protein, 30 aa, Human papillomavirus type 45; identical to SEQ ID NO: 105

SEQ ID NO: 110 - protein, 33 aa, Human papillomavirus type 45
ELTVESSAEDLRTLQQLFLSTLSFVCPWCATNQ

SEQ ID NO: 111 - protein, 20 aa, Human papillomavirus type 45
KCDGRIELTVESSAEDLRTL

SEQ ID NO: 112 - protein, 24 aa, Human papillomavirus type 45
KCDGRIELTVESSAEDLRTLQQLF

SEQ ID NO: 113 - protein, 20 aa, Human papillomavirus type 45
ESSAEDLRTLQQLFLSTLSF

SEQ ID NO: 114 - protein, 23 aa, Human papillomavirus type 45
LRTLQQLFLSTLSFVCPWCATNQ

SEQ ID NO: 115 - protein, 27 aa, Human papillomavirus type 45
KCDGRIELTVESSAEDLRTLQQLFLST

SEQ ID NO: 116 - protein, 20 aa, Human papillomavirus type 45
AEDLRTLQQLFLSTLSFVCP

SEQ ID NO: 117 - protein, 20 aa, Human papillomavirus type 45
LQQLFLSTLSFVCPWCATNQ

SEQ ID NO: 118 - protein, 30 aa, Human papillomavirus type 45
KCDGRIELTVESSAEDLRTLQQLFLSTLSF

SEQ ID NO: 119 - protein, 23 aa, Human papillomavirus type 45
LRTLQQLFLSTLSFVCPWCATNQ

SEQ ID NO: 120 - protein, 32 aa, Human papillomavirus type 18
EVFEFAFKDLFVVYRDSIPHAACHKCIDFYSR

SEQ ID NO: 121 - protein, 35 aa, Human papillomavirus type 18
LLIRCLRCQKPLNPAEKLRHLNEKRRFHNIAGHYR

SEQ ID NO: 122 - protein, 35 aa, Human papillomavirus type 18
PVDLLCHEQLSDSEEENDEIDGVNHQHLPARRAEP

SEQ ID NO: 123 - protein, 35 aa, Human papillomavirus type 45
LLIRCLRCQKPLNPAEKRRHLKDKRRFHSIAGQYR

SEQ ID NO: 124 - protein, 35 aa, Human papillomavirus type 45
IAYAACHKCIDFYSRIRELRYYSNSVYGETLEKIT

SEQ ID NO: 125 - protein, 20 aa, Human papillomavirus type 45
NTCCDQARQERLRRRRETQV

SEQ ID NO: 126 - protein, 158 aa, Human papillomavirus type 18
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 127 - protein, 105 aa, Human papillomavirus type 18
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 128 - protein, 158 aa, Human papillomavirus type 45
MARFDDPKQRPYKLPDLCTELNTSLQDVSIACVYCKATLERTEVYQFAFKDLCIVYRDCIAYAACHKCIDFYSRIRELRYYSNSVYGETLEKITNTELYNLLIRCLRCQKPLNPAEKRRHLKDKRRFHSIAGQYRGQCNTCCDQARQERLRRRRETQV

SEQ ID NO: 129 - protein, 106 aa, Human papillomavirus type 45
MHGPRETLQEIVLHLEPQNELDPVDLLCYEQLSESEEENDEADGVSHAQLPARRAEPQRHKILCVCCKCDGRIELTVESSAEDLRTLQQLFLSTLSFVCPWCATNQ

SEQ ID NO: 130 - protein, 20 aa, Human papillomavirus type 18
KTVLELTEVFEFAFKDLFVV

SEQ ID NO: 131 - protein, 33 aa, Human papillomavirus type 18
FYSRIRELRHYSDSVYGDTLEKLTNTGLYNLLI

SEQ ID NO: 132 - protein, 24 aa, Human papillomavirus type 45
PQNELDPVDLLCYEQLSESEEEND

SEQ ID NO: 133 - protein, 27 aa, Human papillomavirus type 45
PQNELDPVDLLCYEQLSESEEENDEAD

SEQ ID NO: 134 - protein, 149 aa, Human papillomavirus type 33
MFQDTEEKPRTLHDLCQALETTIHNIELQCVECKKPLQRSEVYDFAFADLTVVYREGNPFGICKLCLRFLSKISEYRHYNYSVYGNTLEQTVKKPLNEILIRCIICQRPLCPQEKKRHVDLNKRFHNISGRWAGRCAACWRSRRRETAL

SEQ ID NO: 135 - protein, 97 aa, Human papillomavirus type 33
MRGHKPTLKEYVLDLYPEPTDLYCYEQLSDSSDEDEGLDRPDGQAQPATADYYIVTCCHTCNTTVRLCVNSTASDLRTIQQLLMGTVNIVCPTCAQQ

SEQ ID NO: 136 - protein, 31 aa, Human papillomavirus type 33
FQDTEEKPRTLHDLCQALETTIHNIELQCVE

SEQ ID NO: 137 - protein, 32 aa, Human papillomavirus type 33
MFQDTEEKPRTLHDLCQALETTIHNIELQCVE

SEQ ID NO: 138 - protein, 28 aa, Human papillomavirus type 33
DTEEKPRTLHDLCQALETTIHNIELQCV

SEQ ID NO: 139 - protein, 38 aa, Human papillomavirus type 33
ISEYRHYNYSVYGNTLEQTVKKPLNEILIRCIICQRPL

SEQ ID NO: 140 - protein, 35 aa, Human papillomavirus type 33
VVYREGNPFGICKLCLRFLSKISEYRHYNYSVYGN

SEQ ID NO: 141 - protein, 29 aa, Human papillomavirus type 33
LETTIHNIELQCVECKKPLQRSEVYDFAF

SEQ ID NO: 142 - protein, 35 aa, Human papillomavirus type 33
VECKKPLQRSEVYDFAFADLTVVYREGNPFGICKL

SEQ ID NO: 143 - protein, 30 aa, Human papillomavirus type 33
ALETTIHNIELQCVECKKPLQRSEVYDFAF

SEQ ID NO: 144 - protein, 32 aa, Human papillomavirus type 33
KKPLQRSEVYDFAFADLTVVYREGNPFGICKL

SEQ ID NO: 145 - protein, 35 aa, Human papillomavirus type 33
KKPLQRSEVYDFAFADLTVVYREGNPFGICKLCLR

SEQ ID NO: 146 - protein, 35 aa, Human papillomavirus type 33
REGNPFGICKLCLRFLSKISEYRHYNYSVYGNTLE

SEQ ID NO: 147 - protein, 35 aa, Human papillomavirus type 33
YRHYNYSVYGNTLEQTVKKPLNEILIRCIICQRPL

SEQ ID NO: 148 - protein, 30 aa, Human papillomavirus type 33
LCQALETTIHNIELQCVECKKPLQRSEVYD

SEQ ID NO: 149 - protein, 35 aa, Human papillomavirus type 33
NPFGICKLCLRFLSKISEYRHYNYSVYGNTLEQTV

SEQ ID NO: 150 - protein, 34 aa, Human papillomavirus type 33
RHYNYSVYGNTLEQTVKKPLNEILIRCIICQRPL

SEQ ID NO: 151 - protein, 21 aa, Human papillomavirus type 33
FAFADLTVVYREGNPFGICKL

SEQ ID NO: 152 - protein, 35 aa, Human papillomavirus type 33
GICKLCLRFLSKISEYRHYNYSVYGNTLEQTVKKP

SEQ ID NO: 153 - protein, 26 aa, Human papillomavirus type 33
LETTIHNIELQCVECKKPLQRSEVYD

SEQ ID NO: 154 - protein, 29 aa, Human papillomavirus type 33
SVYGNTLEQTVKKPLNEILIRCIICQRPL

SEQ ID NO: 155 - protein, 28 aa, Human papillomavirus type 33
VECKKPLQRSEVYDFAFADLTVVYREGN

SEQ ID NO: 156 - protein, 27 aa, Human papillomavirus type 33
ALETTIHNIELQCVECKKPLQRSEVYD

SEQ ID NO: 157 - protein, 35 aa, Human papillomavirus type 33
EGNPFGICKLCLRFLSKISEYRHYNYSVYGNTLEQ

SEQ ID NO: 158 - protein, 22 aa, Human papillomavirus type 33
EQTVKKPLNEILIRCIICQRPL

SEQ ID NO: 159 - protein, 30 aa, Human papillomavirus type 33
ISEYRHYNYSVYGNTLEQTVKKPLNEILIR

SEQ ID NO: 160 - protein, 30 aa, Human papillomavirus type 33
IICQRPLCPQEKKRHVDLNKRFHNISGRWA

SEQ ID NO: 161 - protein, 30 aa, Human papillomavirus type 33
DLNKRFHNISGRWAGRCAACWRSRRRETAL

SEQ ID NO: 162 - protein, 35 aa, Human papillomavirus type 33
KKRHVDLNKRFHNISGRWAGRCAACWRSRRRETAL

SEQ ID NO: 163 - protein, 25 aa, Human papillomavirus type 33
IICQRPLCPQEKKRHVDLNKRFHNI

SEQ ID NO: 164 - protein, 32 aa, Human papillomavirus type 33
IICQRPLCPQEKKRHVDLNKRFHNISGRWAGR

SEQ ID NO: 165 - protein, 28 aa, Human papillomavirus type 33
NKRFHNISGRWAGRCAACWRSRRRETAL

SEQ ID NO: 166 - protein, 35 aa, Human papillomavirus type 33
MRGHKPTLKEYVLDLYPEPTDLYCYEQLSDSSDED

SEQ ID NO: 167 - protein, 35 aa, Human papillomavirus type 33
EQLSDSSDEDEGLDRPDGQAQPATADYYIVTCCHT

SEQ ID NO: 168 - protein, 20 aa, Human papillomavirus type 33
LYCYEQLSDSSDEDEGLDRP

SEQ ID NO: 169 - protein, 28 aa, Human papillomavirus type 33
RGHKPTLKEYVLDLYPEPTDLYCYEQLS

SEQ ID NO: 170 - protein, 35 aa, Human papillomavirus type 33
KPTLKEYVLDLYPEPTDLYCYEQLSDSSDEDEGLD

SEQ ID NO: 171 - protein, 28 aa, Human papillomavirus type 33
DEDEGLDRPDGQAQPATADYYIVTCCHT

SEQ ID NO: 172 - protein, 30 aa, Human papillomavirus type 33
MRGHKPTLKEYVLDLYPEPTDLYCYEQLSD

SEQ ID NO: 173 - protein, 30 aa, Human papillomavirus type 33
PEPTDLYCYEQLSDSSDEDEGLDRPDGQAQ

SEQ ID NO: 174 - protein, 30 aa, Human papillomavirus type 33
DLYCYEQLSDSSDEDEGLDRPDGQAQPATA

SEQ ID NO: 175 - protein, 24 aa, Human papillomavirus type 33
GLDRPDGQAQPATADYYIVTCCHT

SEQ ID NO: 176 - protein, 31 aa, Human papillomavirus type 33
MRGHKPTLKEYVLDLYPEPTDLYCYEQLSDS

SEQ ID NO: 177 - protein, 21 aa, Human papillomavirus type 33
ASDLRTIQQLLMGTVNIVCPT

SEQ ID NO: 178 - protein, 25 aa, Human papillomavirus type 33
HTCNTTVRLCVNSTASDLRTIQQLL

SEQ ID NO: 179 - protein, 25 aa, Human papillomavirus type 33
ASDLRTIQQLLMGTVNIVCPTCAQQ

SEQ ID NO: 180 - protein, 24 aa, Human papillomavirus type 33
HTCNTTVRLCVNSTASDLRTIQQL

SEQ ID NO: 181 - protein, 28 aa, Human papillomavirus type 33
HTCNTTVRLCVNSTASDLRTIQQLLMGT

SEQ ID NO: 182 - protein, 20 aa, Human papillomavirus type 33
TIQQLLMGTVNIVCPTCAQQ

SEQ ID NO: 183 - protein, 22 aa, Human papillomavirus type 33
DLRTIQQLLMGTVNIVCPTCAQ

SEQ ID NO: 184 - protein, 29 aa, Human papillomavirus type 33
HTCNTTVRLCVNSTASDLRTIQQLLMGTV

SEQ ID NO: 185 - protein, 21 aa, Human papillomavirus type 33
PATADYYIVTCCHTCNTTVRL

SEQ ID NO: 186 - protein, 148 aa, Human papillomavirus type 52
MFEDPATRPRTLHELCEVLEESVHEIRLQCVQCKKELQRREVYKFLFTDLRIVYRDNNPYGVCIMCLRFLSKISEYRHYQYSLYGKTLEERVKKPLSEITIRCIICQTPLCPEEKERHVNANKRFHNIMGRWTGRCSECWRPRPVTQV

SEQ ID NO: 187 - protein, 99 aa, Human papillomavirus type 52
MRGDKATIKDYILDLQPETTDLHCYEQLGDSSDEEDTDGVDRPDGQAEQATSNYYIVTYCHSCDSTLRLCIHSTATDLRTLQQMLLGTLQVVCPGCARL

SEQ ID NO: 188 - protein, 32 aa, Human papillomavirus type 52
MFEDPATRPRTLHELCEVLEESVHEIRLQCVQ

SEQ ID NO: 189 - protein, 31 aa, Human papillomavirus type 52
FEDPATRPRTLHELCEVLEESVHEIRLQCVQ

SEQ ID NO: 190 - protein, 28 aa, Human papillomavirus type 52
PATRPRTLHELCEVLEESVHEIRLQCVQ

SEQ ID NO: 191 - protein, 31 aa, Human papillomavirus type 52
LEESVHEIRLQCVQCKKELQRREVYKFLFTD

SEQ ID NO: 192 - protein, 29 aa, Human papillomavirus type 52
SKISEYRHYQYSLYGKTLEERVKKPLSEI

SEQ ID NO: 193 - protein, 22 aa, Human papillomavirus type 52
GVCIMCLRFLSKISEYRHYQYS

SEQ ID NO: 194 - protein, 29 aa, Human papillomavirus type 52
SLYGKTLEERVKKPLSEITIRCIICQTPL

SEQ ID NO: 195 - protein, 25 aa, Human papillomavirus type 52
DLRIVYRDNNPYGVCIMCLRFLSKI

SEQ ID NO: 196 - protein, 23 aa, Human papillomavirus type 52
REVYKFLFTDLRIVYRDNNPYGV

SEQ ID NO: 197 - protein, 31 aa, Human papillomavirus type 52
FLSKISEYRHYQYSLYGKTLEERVKKPLSEI

SEQ ID NO: 198 - protein, 25 aa, Human papillomavirus type 52
RIVYRDNNPYGVCIMCLRFLSKISE

SEQ ID NO: 199 - protein, 30 aa, Human papillomavirus type 52
VLEESVHEIRLQCVQCKKELQRREVYKFLF

SEQ ID NO: 200 - protein, 34 aa, Human papillomavirus type 52
VQCKKELQRREVYKFLFTDLRIVYRDNNPYGVCI

SEQ ID NO: 201 - protein, 22 aa, Human papillomavirus type 52
EERVKKPLSEITIRCIICQTPL

SEQ ID NO: 202 - protein, 30 aa, Human papillomavirus type 52
KISEYRHYQYSLYGKTLEERVKKPLSEITI

SEQ ID NO: 203 - protein, 32 aa, Human papillomavirus type 52
LEESVHEIRLQCVQCKKELQRREVYKFLFTDL

SEQ ID NO: 204 - protein, 25 aa, Human papillomavirus type 52
YGVCIMCLRFLSKISEYRHYQYSLY

SEQ ID NO: 205 - protein, 27 aa, Human papillomavirus type 52
NKRFHNIMGRWTGRCSECWRPRPVTQV

SEQ ID NO: 206 - protein, 24 aa, Human papillomavirus type 52
IICQTPLCPEEKERHVNANKRFHN

SEQ ID NO: 207 - protein, 29 aa, Human papillomavirus type 52
NANKRFHNIMGRWTGRCSECWRPRPVTQV

SEQ ID NO: 208 - protein, 35 aa, Human papillomavirus type 52
EKERHVNANKRFHNIMGRWTGRCSECWRPRPVTQV

SEQ ID NO: 209 - protein, 37 aa, Human papillomavirus type 52
EQLGDSSDEEDTDGVDRPDGQAEQATSNYYIVTYCHS

SEQ ID NO: 210 - protein, 35 aa, Human papillomavirus type 52
MRGDKATIKDYILDLQPETTDLHCYEQLGDSSDEE

SEQ ID NO: 211 - protein, 22 aa, Human papillomavirus type 52
EQATSNYYIVTYCHSCDSTLRL

SEQ ID NO: 212 - protein, 25 aa, Human papillomavirus type 52
GQAEQATSNYYIVTYCHSCDSTLRL

SEQ ID NO: 213 - protein, 30 aa, Human papillomavirus type 52
LHCYEQLGDSSDEEDTDGVDRPDGQAEQAT

SEQ ID NO: 214 - protein, 32 aa, Human papillomavirus type 52
LGDSSDEEDTDGVDRPDGQAEQATSNYYIVTY

SEQ ID NO: 215 - protein, 35 aa, Human papillomavirus type 52
RGDKATIKDYILDLQPETTDLHCYEQLGDSSDEED

SEQ ID NO: 216 - protein, 32 aa, Human papillomavirus type 52
HSCDSTLRLCIHSTATDLRTLQQMLLGTLQVV

SEQ ID NO: 217 - protein, 21 aa, Human papillomavirus type 52
RTLQQMLLGTLQVVCPGCARL

SEQ ID NO: 218 - protein, 25 aa, Human papillomavirus type 52
HSCDSTLRLCIHSTATDLRTLQQML

SEQ ID NO: 219 - protein, 28 aa, Human papillomavirus type 52
HSTATDLRTLQQMLLGTLQVVCPGCARL

SEQ ID NO: 220 - protein, 20 aa, Human papillomavirus type 52
SCDSTLRLCIHSTATDLRTL

SEQ ID NO: 221 - protein, 149 aa, Human papillomavirus type 31
MFKNPAERPRKLHELSSALEIPYDELRLNCVYCKGQLTETEVLDFAFTDLTIVYRDDTPHGVCTKCLRFYSKVSEFRWYRYSVYGTTLEKLTNKGICDLLIRCITCQRPLCPEEKQRHLDKKKRFHNIGGRWTGRCIACWRRPRTETQV

SEQ ID NO: 222 - protein, 98 aa, Human papillomavirus type 31
MRGETPTLQDYVLDLQPEATDLHCYEQLPDSSDEEDVIDSPAGQAEPDTSNYNIVTFCCQCKSTLRLCVQSTQVDIRILQELLMGSFGIVCPNCSTRL

SEQ ID NO: 223 - protein, 32 aa, Human papillomavirus type 31
LHELSSALEIPYDELRLNCVYCKGQLTETEVL

SEQ ID NO: 224 - protein, 31 aa, Human papillomavirus type 31
FKNPAERPRKLHELSSALEIPYDELRLNCVY

SEQ ID NO: 225 - protein, 25 aa, Human papillomavirus type 31
MFKNPAERPRKLHELSSALEIPYDE

SEQ ID NO: 226 - protein, 25 aa, Human papillomavirus type 31
LEIPYDELRLNCVYCKGQLTETEVL

SEQ ID NO: 227 - protein, 26 aa, Human papillomavirus type 31
LTETEVLDFAFTDLTIVYRDDTPHGV

SEQ ID NO: 228 - protein, 35 aa, Human papillomavirus type 31
HGVCTKCLRFYSKVSEFRWYRYSVYGTTLEKLTNK

SEQ ID NO: 229 - protein, 21 aa, Human papillomavirus type 31
VYRDDTPHGVCTKCLRFYSKV

SEQ ID NO: 230 - protein, 25 aa, Human papillomavirus type 31
WYRYSVYGTTLEKLTNKGICDLLIR

SEQ ID NO: 231 - protein, 35 aa, Human papillomavirus type 31
LTIVYRDDTPHGVCTKCLRFYSKVSEFRWYRYSVY

SEQ ID NO: 232 - protein, 35 aa, Human papillomavirus type 31
SKVSEFRWYRYSVYGTTLEKLTNKGICDLLIRCIT

SEQ ID NO: 233 - protein, 25 aa, Human papillomavirus type 31
HGVCTKCLRFYSKVSEFRWYRYSVY

SEQ ID NO: 234 - protein, 32 aa, Human papillomavirus type 31
ITCQRPLCPEEKQRHLDKKKRFHNIGGRWTGR

SEQ ID NO: 235 - protein, 30 aa, Human papillomavirus type 31
DKKKRFHNIGGRWTGRCIACWRRPRTETQV

SEQ ID NO: 236 - protein, 26 aa, Human papillomavirus type 31
RFHNIGGRWTGRCIACWRRPRTETQV

SEQ ID NO: 237 - protein, 25 aa, Human papillomavirus type 31
ITCQRPLCPEEKQRHLDKKKRFHNI

SEQ ID NO: 238 - protein, 29 aa, Human papillomavirus type 31
SDEEDVIDSPAGQAEPDTSNYNIVTFCCQ

SEQ ID NO: 239 - protein, 29 aa, Human papillomavirus type 31
PEATDLHCYEQLPDSSDEEDVIDSPAGQA

SEQ ID NO: 240 - protein, 29 aa, Human papillomavirus type 31
RGETPTLQDYVLDLQPEATDLHCYEQLPD

SEQ ID NO: 241 - protein, 34 aa, Human papillomavirus type 31
HCYEQLPDSSDEEDVIDSPAGQAEPDTSNYNIVT

SEQ ID NO: 242 - protein, 35 aa, Human papillomavirus type 31
MRGETPTLQDYVLDLQPEATDLHCYEQLPDSSDEE

SEQ ID NO: 243 - protein, 23 aa, Human papillomavirus type 31
EDVIDSPAGQAEPDTSNYNIVTF

SEQ ID NO: 244 - protein, 29 aa, Human papillomavirus type 31
KSTLRLCVQSTQVDIRILQELLMGSFGIV

SEQ ID NO: 245 - protein, 21 aa, Human papillomavirus type 31
RILQELLMGSFGIVCPNCSTR

SEQ ID NO: 246 - protein, 37 aa, Human papillomavirus type 31
KSTLRLCVQSTQVDIRILQELLMGSFGIVCPNCSTRL

SEQ ID NO: 247 - protein, 22 aa, Human papillomavirus type 31
VQSTQVDIRILQELLMGSFGIV

SEQ ID NO: 248 - protein, 21 aa, Human papillomavirus type 31
KSTLRLCVQSTQVDIRILQEL

SEQ ID NO: 249 - protein, 25 aa, Human papillomavirus type 31
VQSTQVDIRILQELLMGSFGIVCPN

SEQ ID NO: 250 - protein, 32 aa, Human papillomavirus type 31
KSTLRLCVQSTQVDIRILQELLMGSFGIVCPN